(19) United States
(12) Patent Application Publication
(10) Pub. No.: US 2025/0229066 A1
(43) Pub. Date: Jul. 17, 2025

(54) SHUNT DEVICES INCLUDING TISSUE CAPTURE FEATURES

(71) Applicant: EDWARDS LIFESCIENCES CORPORATION, Irvine, CA (US)

(72) Inventors: Amirsepehr Azimian, Costa Mesa, CA (US); Lynn T. Dang, Huntington Beach, CA (US); Yevgeniy Vyatcheslavovich Fedotov, Ladera Ranch, CA (US); Daniel Vincent Llanes, Huntington Beach, CA (US); Ramon Aguilar, JR., Corona, CA (US); Scott Louis Pool, Laguna Hills, CA (US); Sandra Ahide Alcantar Chavez, Anaheim, CA (US); Charissa Seid, Costa Mesa, CA (US)

(73) Assignee: EDWARDS LIFESCIENCES CORPORATION, Irvine, CA (US)

(21) Appl. No.: 19/098,869

(22) Filed: Apr. 2, 2025

Related U.S. Application Data
(63) Continuation of application No. PCT/US2023/034391, filed on Oct. 3, 2023.
(60) Provisional application No. 63/378,178, filed on Oct. 3, 2022.

(51) Int. Cl. A61M 27/00

(52) U.S. Cl. CPC A61M 27/002; A61M 2205/0266; A61M 2205/32; A61M 2210/125; A61M 2230/30

(57) ABSTRACT

A shunt device is configured to be inserted into a puncture in a tissue wall that defines a horizontal reference plane. The shunt device includes a shunt body. The shunt body includes a central flow tube extending from a first axial end to a second axial end and defining a central axis therethrough that is angled from a reference axis extending perpendicular through the horizontal reference plane; a flow path extending through the central flow tube; and a plurality of arms extending outward from the central flow tube and configured to secure the shunt device to the tissue wall. The plurality of arms includes a first distal arm and a second distal arm attached to the first axial end of the central flow tube and a first proximal arm and a second proximal arm attached to the second axial end of the central flow tube.

### CROSS-REFERENCE TO RELATED APPLICATION(S)

This application is a continuation of International Application No. PCT/US2023/034391, filed Oct. 3, 2023, which claims the benefit of U.S. Provisional Application No. 63/378,178, filed Oct. 3, 2022, the disclosures of which are hereby incorporated by reference in their entireties.

### BACKGROUND

The present disclosure relates generally to implantable devices and more specifically to cardiovascular shunt devices.

Shunt devices can be positioned in the heart to shunt blood between the left atrium and the right atrium to reduce pressure in the left atrium. The left atrium can experience elevated pressure due to abnormal heart conditions caused by age and/or disease. For example, shunt devices can be used to treat patients with heart failure (also known as congestive heart failure). Shunt devices can be positioned in the septal wall between the left atrium and the right atrium to shunt blood from the left atrium into the right atrium, thus reducing the pressure in the left atrium.

### SUMMARY

In one example, a shunt device is configured to be inserted into a puncture in a tissue wall that defines a horizontal reference plane. The shunt device includes a shunt body formed of a plurality of struts. The shunt body includes a central flow tube extending from a first axial end to a second axial end and defining a central axis therethrough that is angled from a reference axis extending perpendicular through the horizontal reference plane; a flow path extending through the central flow tube; and a plurality of arms extending outward from the central flow tube and configured to secure the shunt device to the tissue wall. Each arm of the plurality of arms extends from the central flow tube to a terminal end. The plurality of arms includes a first distal arm and a second distal arm attached to the first axial end of the central flow tube and a first proximal arm and a second proximal arm attached to the second axial end of the central flow tube. At least one of the plurality of arms includes a lengthened portion adjacent a respective terminal end.

In another example, a shunt device is configured to be inserted into a puncture in a tissue wall that defines a horizontal reference plane. The shunt device includes a shunt body formed of a plurality of struts. The shunt body includes a central flow tube extending from a first axial end to a second axial end and defined by an opposed pair of side portions that extend laterally between an opposed pair of end portions; a flow path extending through the central flow tube; and a plurality of arms extending outward from the central flow tube and configured to secure the shunt device to the tissue wall. The plurality of arms includes a first distal arm and a second distal arm attached to the first axial end of the central flow tube and a first proximal arm and a second proximal arm attached to the second axial end of the central flow tube. The shunt device further includes one or more tabs extending outward from the central flow tube and configured to prevent the shunt device from displacing through the puncture.

In another example, a shunt device is configured to be inserted into a puncture in a tissue wall that defines a horizontal reference plane. The shunt device includes a shunt body formed of a plurality of struts. The shunt body includes a central flow tube extending from a first axial end to a second axial end and defined by an opposed pair of side portions that extend laterally between an opposed pair of end portions; a flow path extending through the central flow tube; and a plurality of arms extending outward from the central flow tube and configured to secure the shunt device to the tissue wall. The plurality of arms includes a first distal arm and a second distal arm attached to the first axial end of the central flow tube and a first proximal arm and a second proximal arm attached to the second axial end of the central flow tube. The shunt device further includes a deflectable projection connected to at least one of the plurality of arms.

In another example, a shunt device is configured to be inserted into a puncture in a tissue wall that defines a horizontal reference plane. The shunt device includes a shunt body formed of a plurality of struts. The shunt body includes a central flow tube extending from a first axial end to a second axial end and defined by an opposed pair of side portions that extend laterally between an opposed pair of end portions; a flow path extending through the central flow tube; and a plurality of arms extending outward from the central flow tube and configured to secure the shunt device to the tissue wall. The plurality of arms includes a first distal arm and a second distal arm attached to the first axial end of the central flow tube and a first proximal arm and a second proximal arm attached to the second axial end of the central flow tube. The shunt device further includes a secondary arm associated with at least one of the plurality of arms.

In another example, a shunt device is configured to be inserted into a puncture in a tissue wall that defines a horizontal reference plane. The shunt device includes a shunt body formed of a plurality of struts. The shunt body includes a central flow tube extending from a first axial end to a second axial end and defining a central axis therethrough that is angled from a reference axis extending perpendicular through the horizontal reference plane; a flow path extending through the central flow tube; and a plurality of arms extending outward from the central flow tube and configured to secure the shunt device to the tissue wall. The plurality of arms includes a first distal arm and a second distal arm attached to the first axial end of the central flow tube and a first proximal arm and a second proximal arm attached to the second axial end of the central flow tube. At least one of the plurality of arms includes two or more split arm portions.

In another example, a shunt device is configured to be inserted into a puncture in a tissue wall that defines a horizontal reference plane. The shunt device includes a shunt body formed of a plurality of struts. The shunt body includes a central flow tube extending from a first axial end to a second axial end and defining a central axis therethrough that is angled from a reference axis extending perpendicular through the horizontal reference plane; a flow path extending through the central flow tube; and a plurality of arms extending outward from the central flow tube and configured to secure the shunt device to the tissue wall. The shunt device further includes a sensor attached to one of the plurality of arms of the shunt body at a mating interface such that the sensor and the one of the plurality of arms are interconnected.

### BRIEF DESCRIPTION OF THE DRAWINGS

### Anatomy of Heart H and Vasculature V

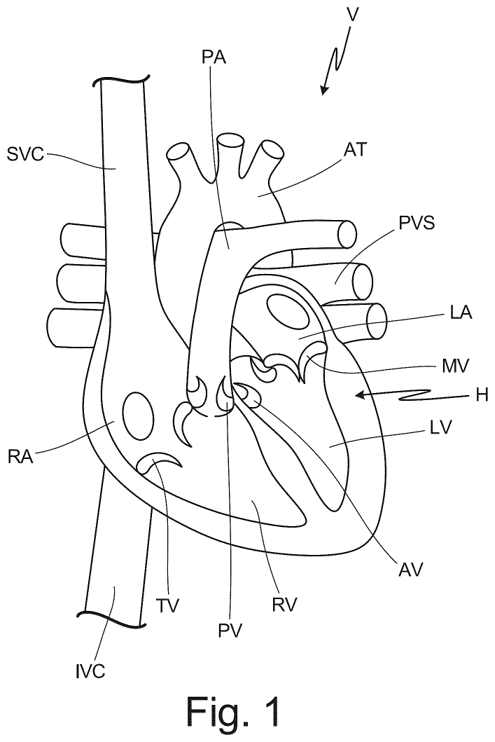

FIG. 1 is a schematic diagram of a heart and vasculature.

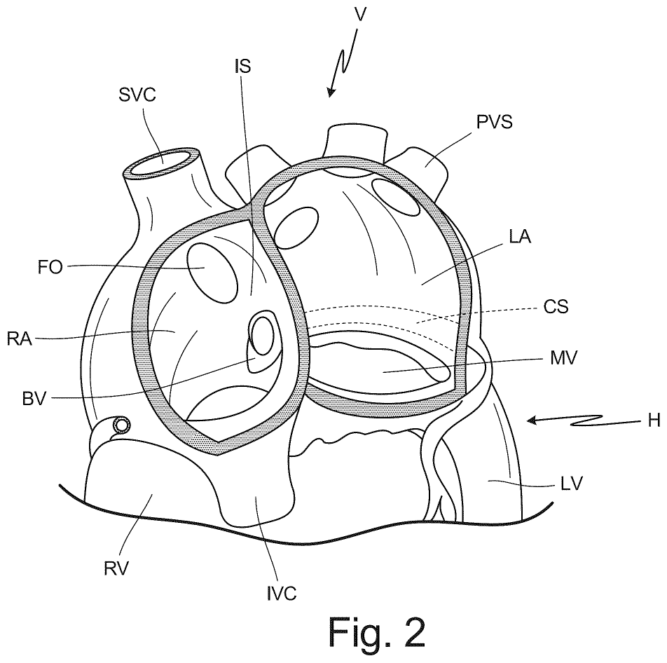

FIG. 2 is a schematic cross-sectional view of the heart.

### Shunt Devices 100 and 100′

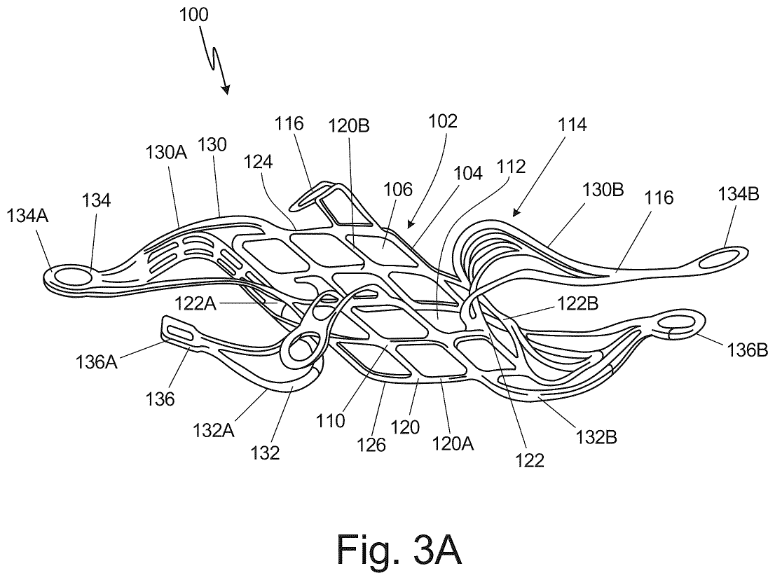

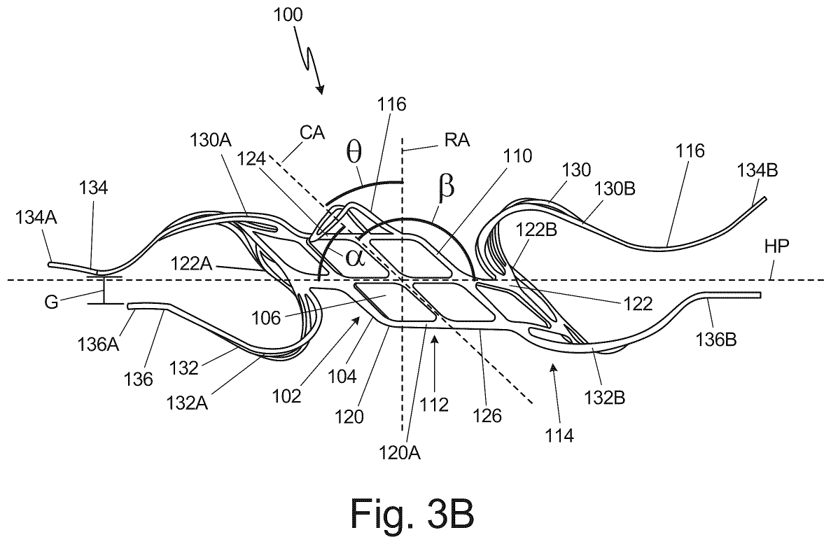

FIG. 3A is a perspective view of a shunt device.

FIG. 3B is a side view of the shunt device.

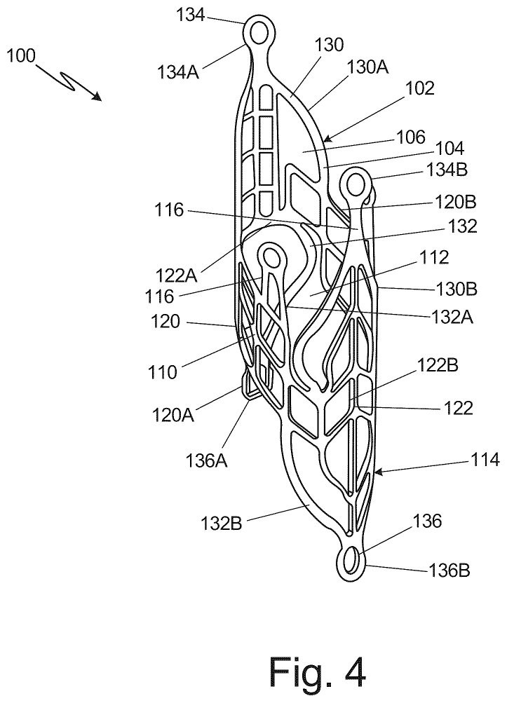

FIG. 4 is a perspective view of the shunt device in a collapsed configuration.

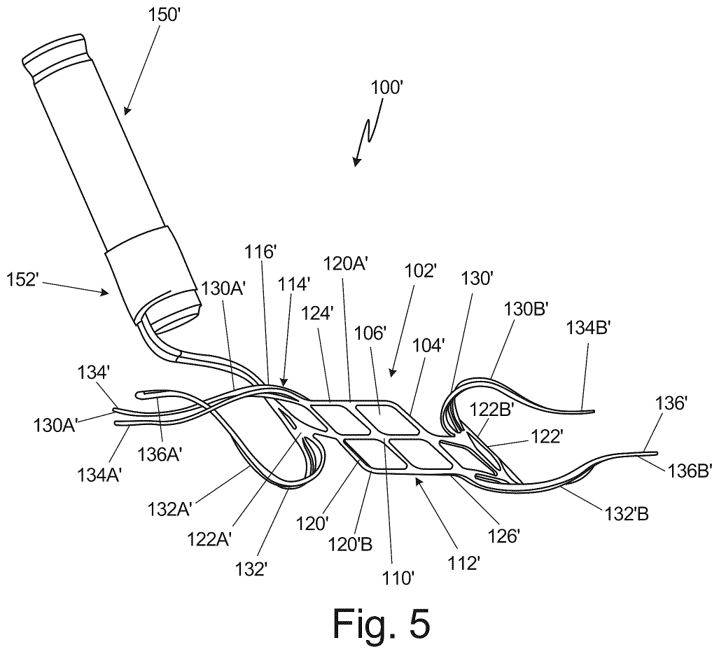

FIG. 5 is a perspective view of a shunt device including a sensor.

### Delivery Catheter 200

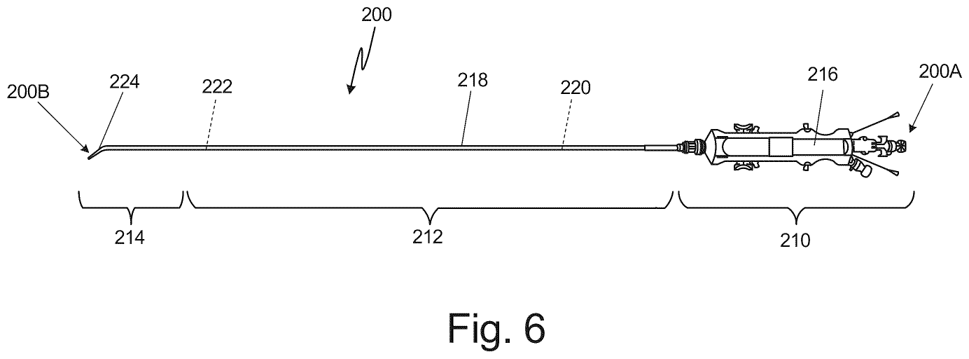

FIG. 6 is a side view of a delivery catheter.

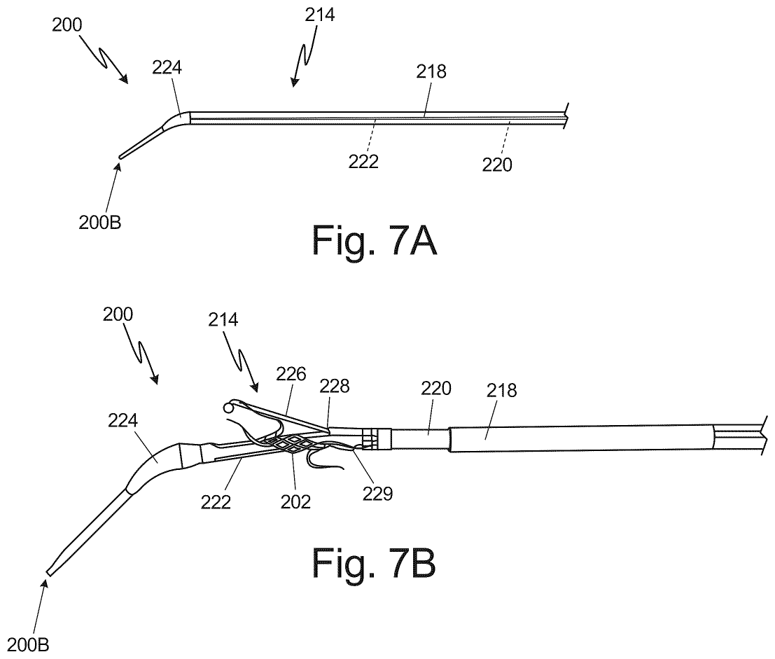

FIG. 7A is a side view of a distal portion of the delivery catheter in a sheathed state.

FIG. 7B is a side view of the distal portion of the delivery catheter in an unsheathed state.

### Delivery Method 300

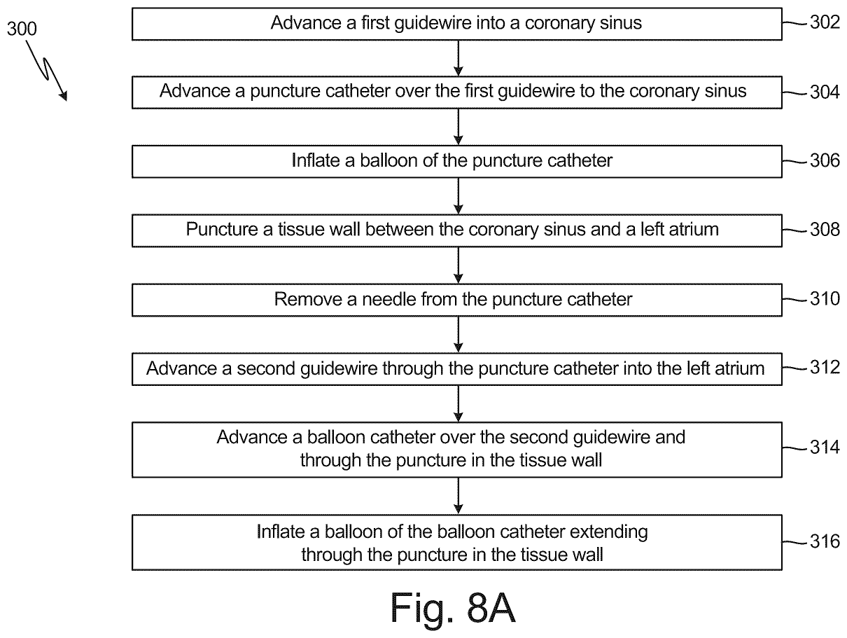

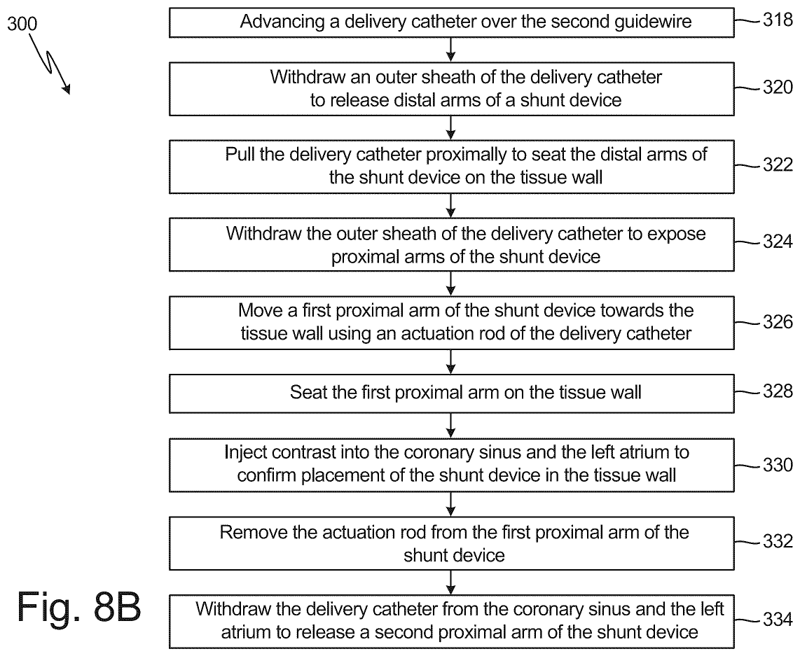

FIG. 8A is a flow chart showing steps for creating a puncture in a tissue wall between a coronary sinus and a left atrium.

FIG. 8B is a flow chart showing steps for implanting a shunt device in the tissue wall between the coronary sinus and the left atrium.

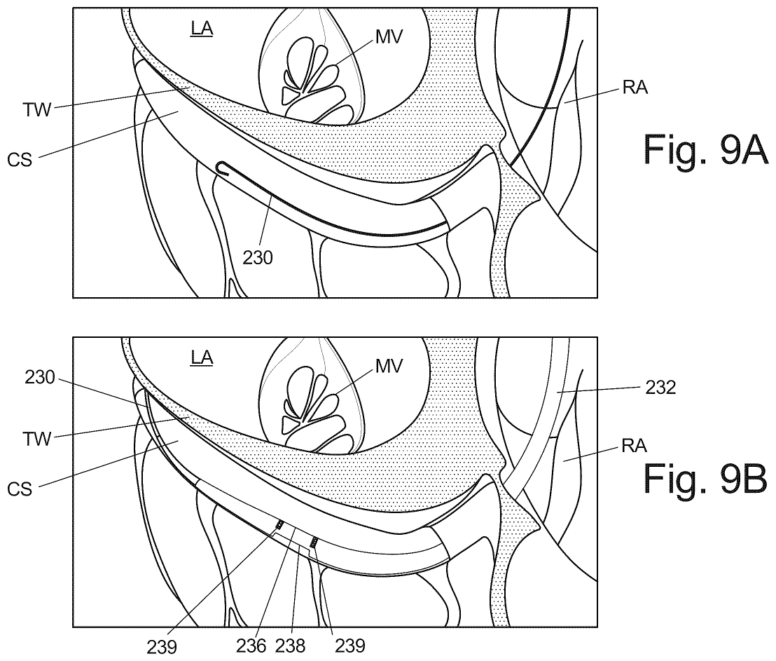

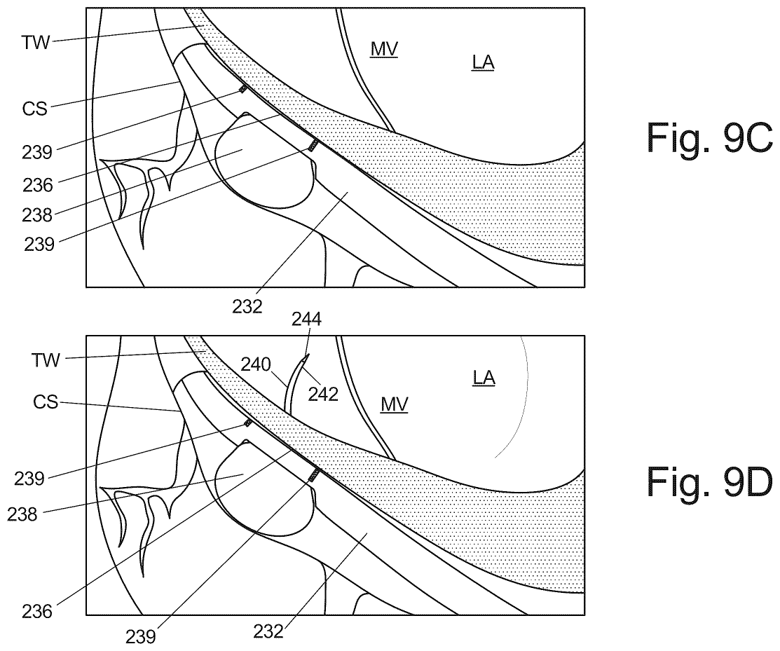

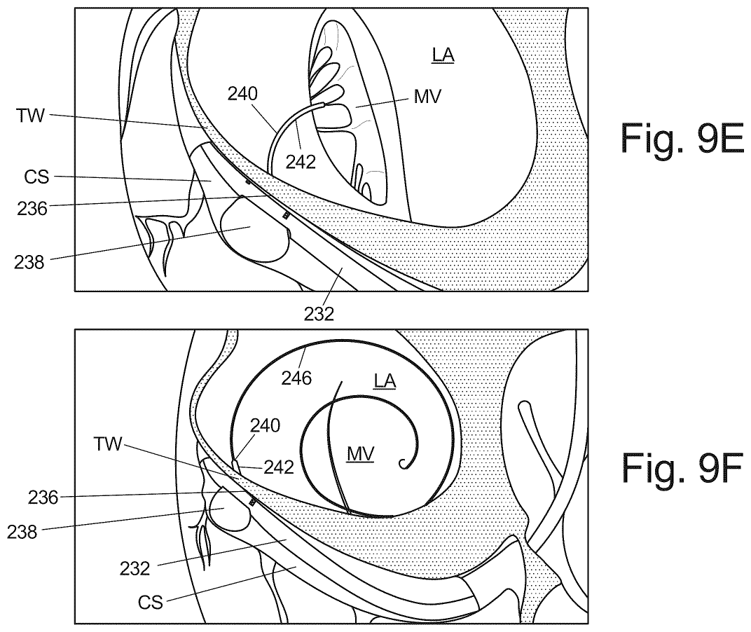

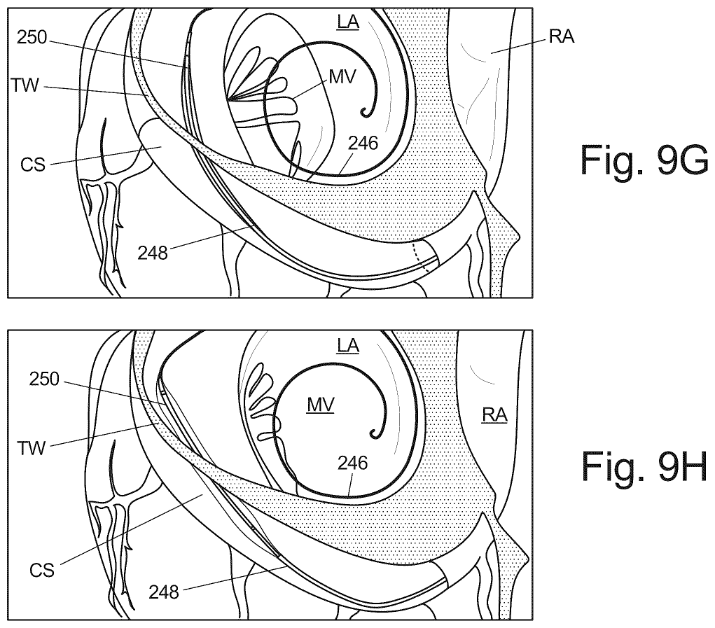

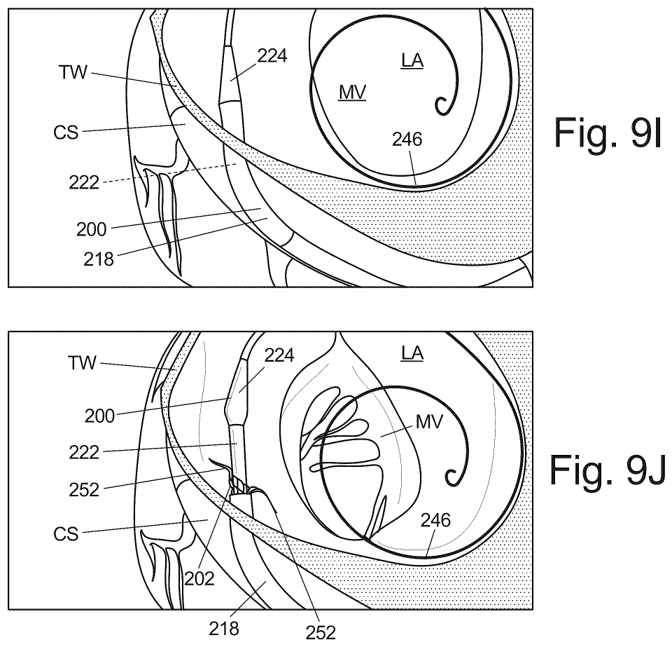

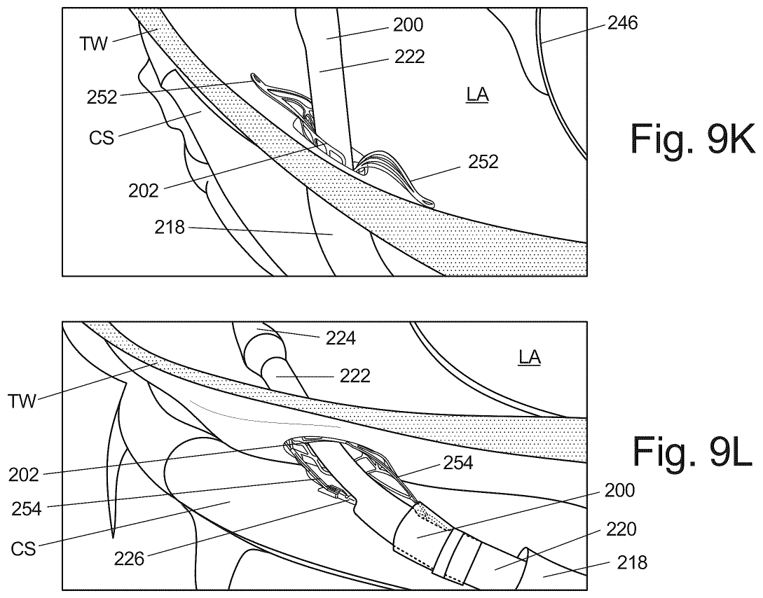

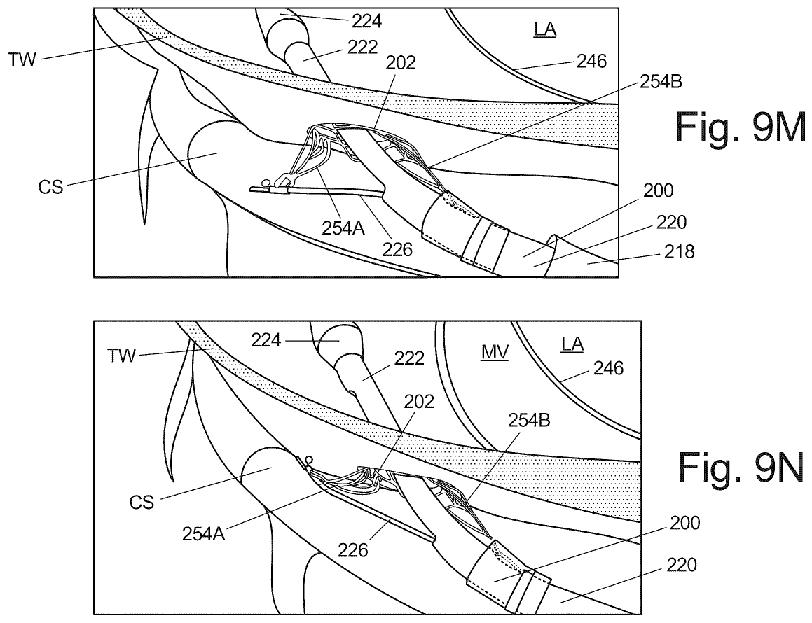

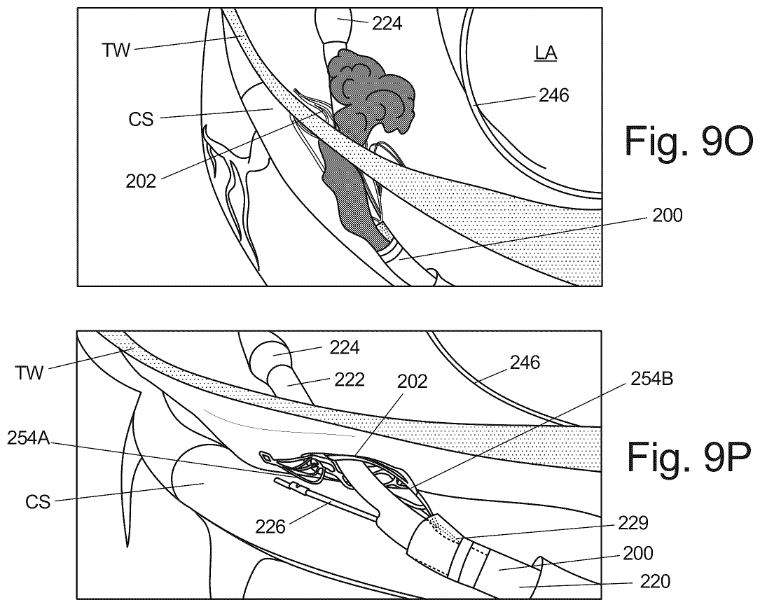

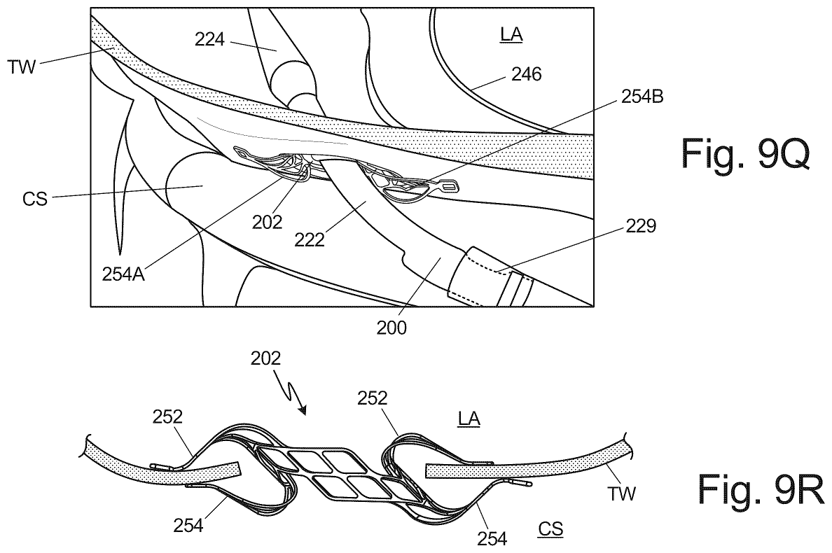

FIGS. 9A-9R are schematic views showing the steps for implanting a shunt device in the tissue wall between the coronary sinus and the left atrium.

### Shunt Device 400

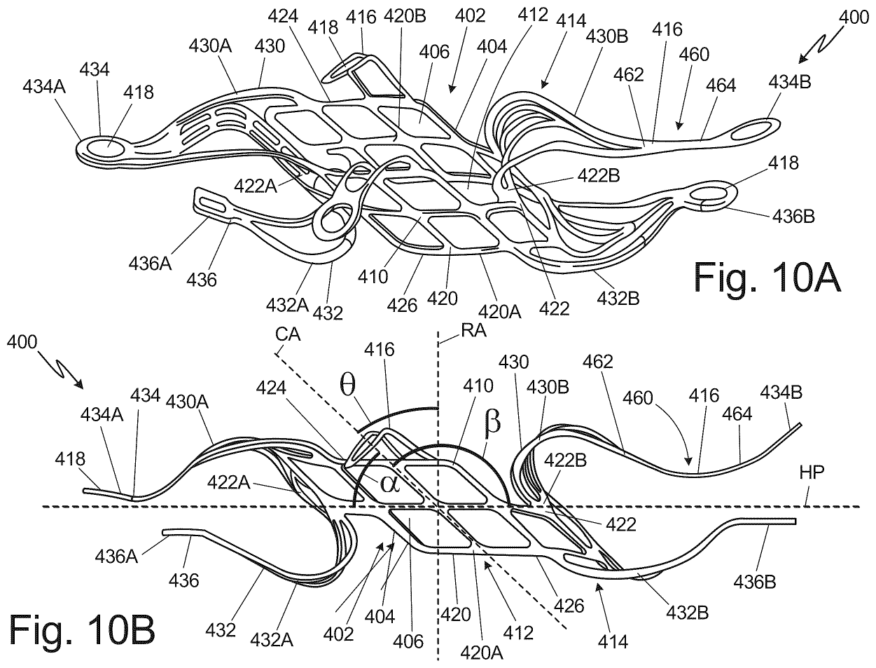

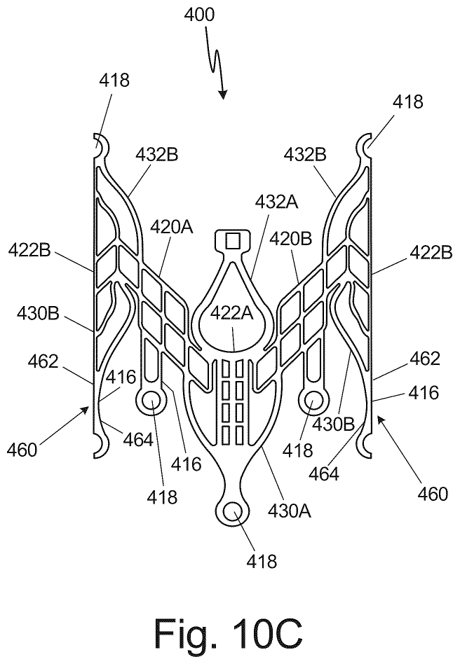

FIG. 10A is a perspective view of a shunt device including a lengthened portion.

FIG. 10B is a side view of the shunt device including the lengthened portion.

FIG. 10C is a flattened view of the shunt device including the lengthened portion.

### Shunt Devices 500 and 500′

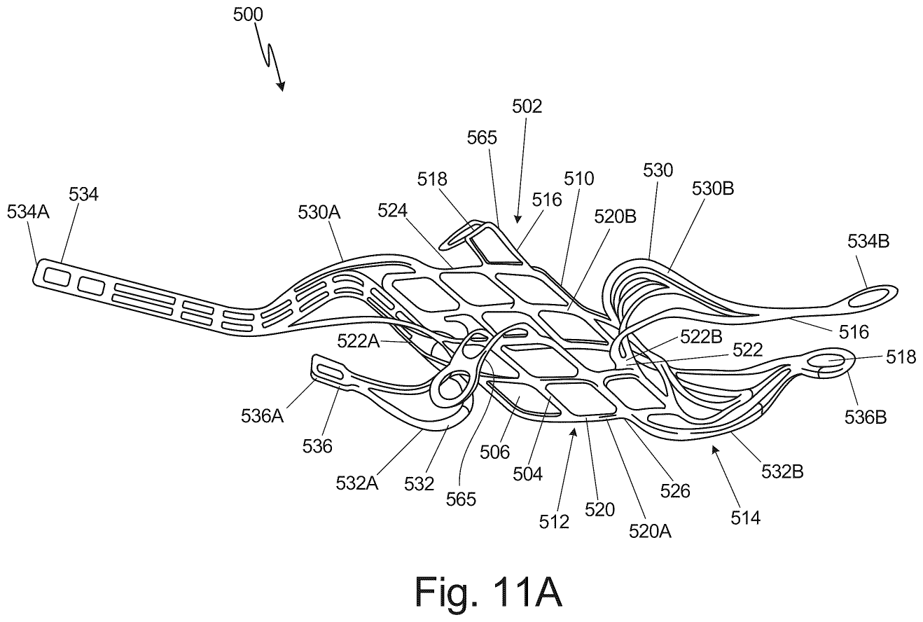

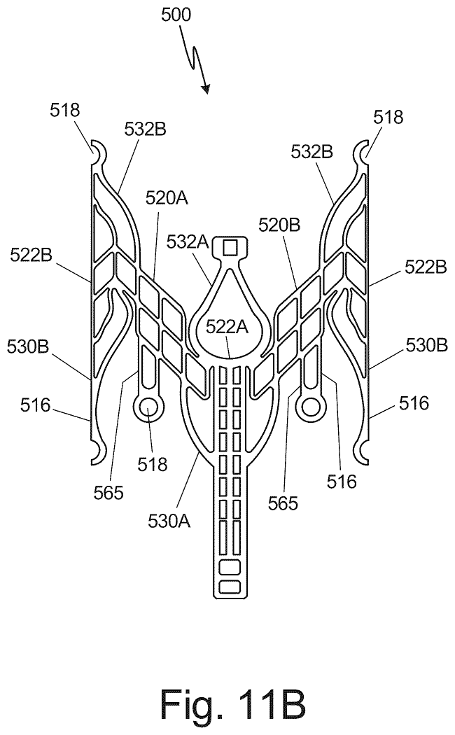

FIG. 11A is a perspective view of a first example of a shunt device including tabs.

FIG. 11B is a flattened view of the first example of the shunt device including the tabs.

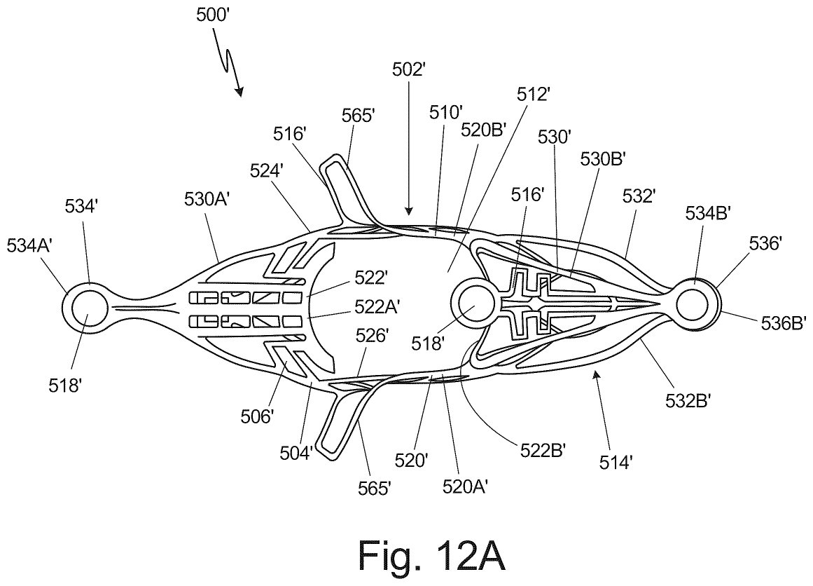

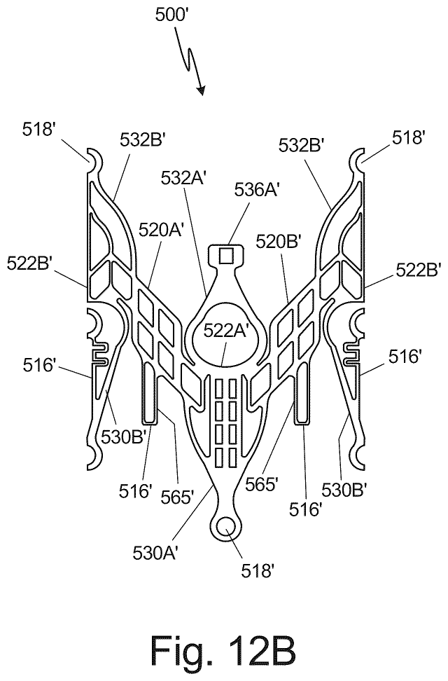

FIG. 12A is a top view of a second example of a shunt device including tabs.

FIG. 12B is a flattened view of the second example of the shunt device including the tabs.

### Shunt Devices 600, 600′, and 600″

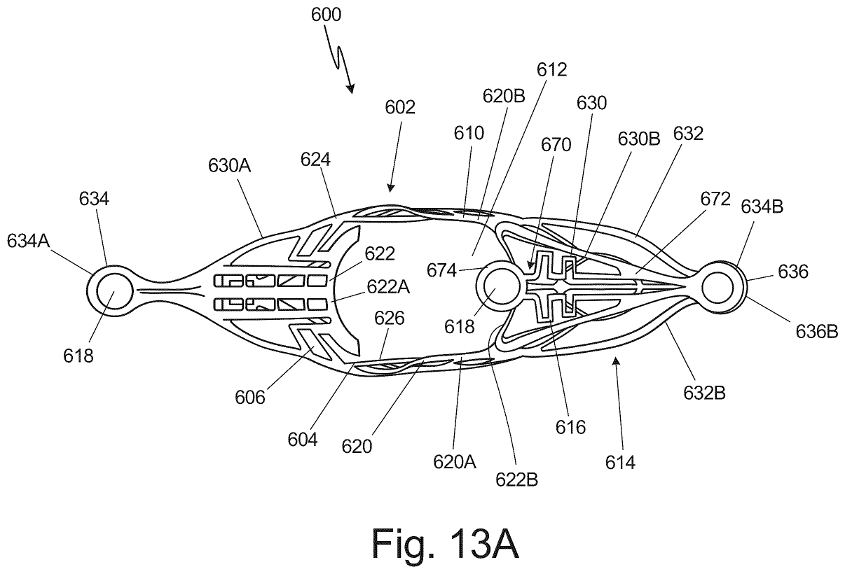

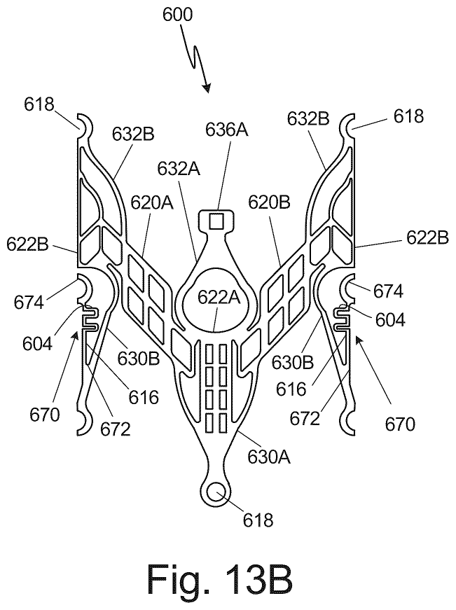

FIG. 13A is a top view of a first example of a shunt device including a deflectable projection.

FIG. 13B is a flattened view of the first example of the shunt device including the deflectable projection.

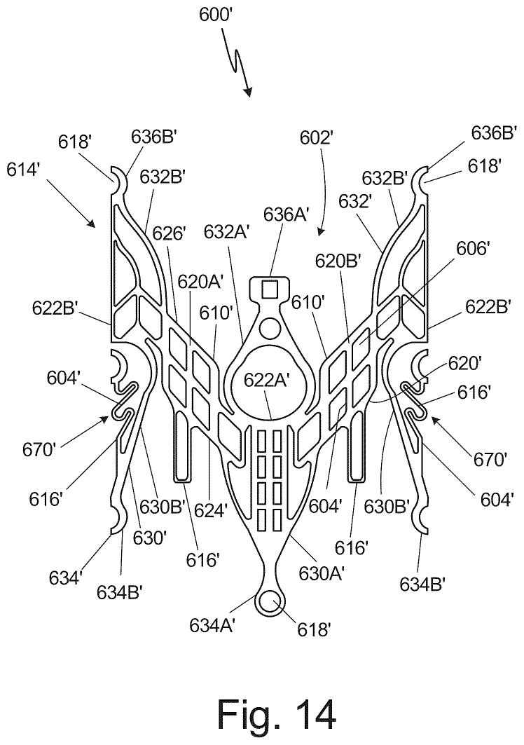

FIG. 14 is a flattened view of a second example of a shunt device including a deflectable projection.

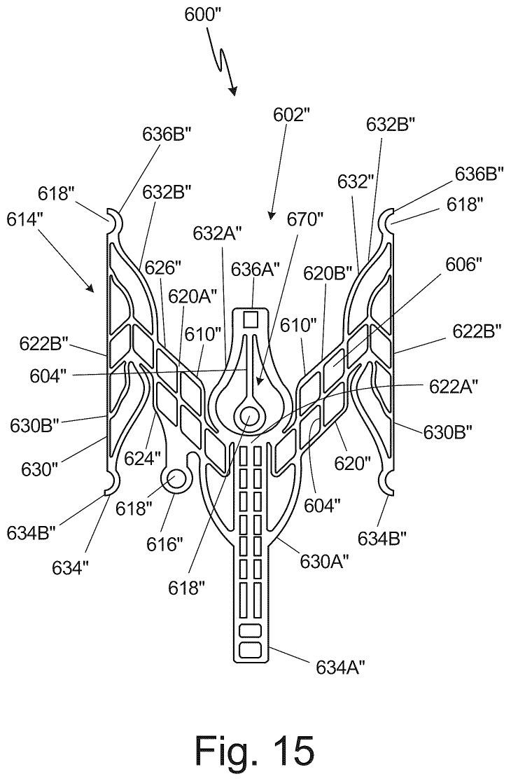

FIG. 15 is a flattened view of a third example of a shunt device including a deflectable projection.

FIG. 16A is a schematic view of a shunt device including a deflectable projection in a relaxed state.

FIG. 16B is a schematic view of the shunt device including the deflectable projection in a deflected state.

### Shunt Devices 700 and 700′

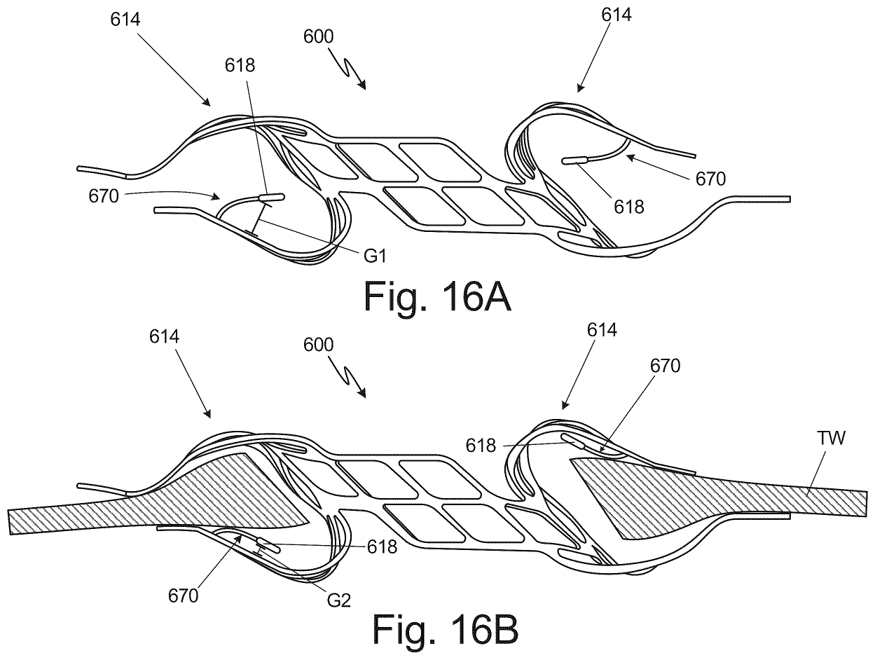

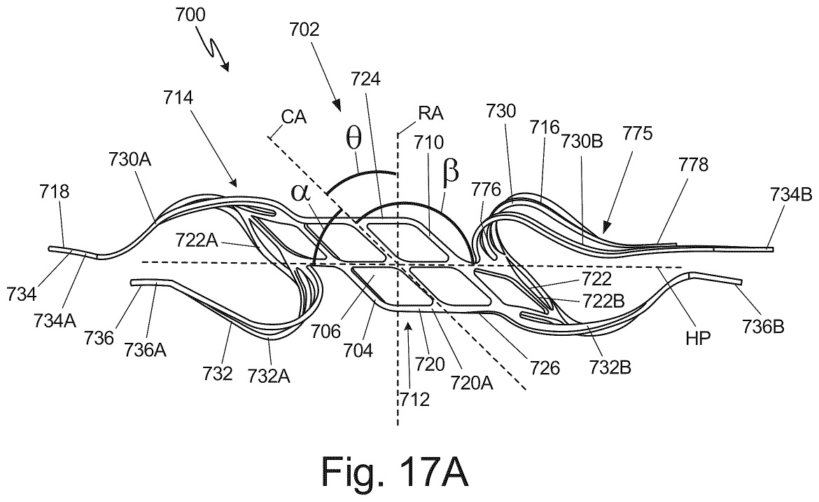

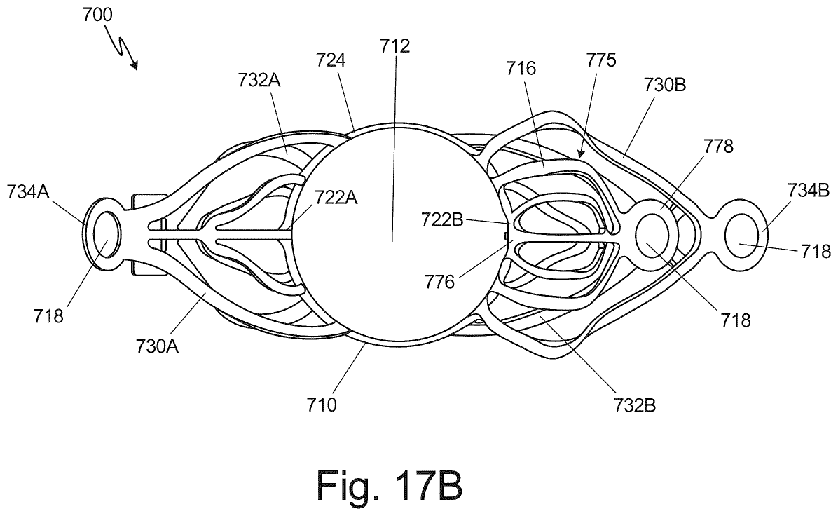

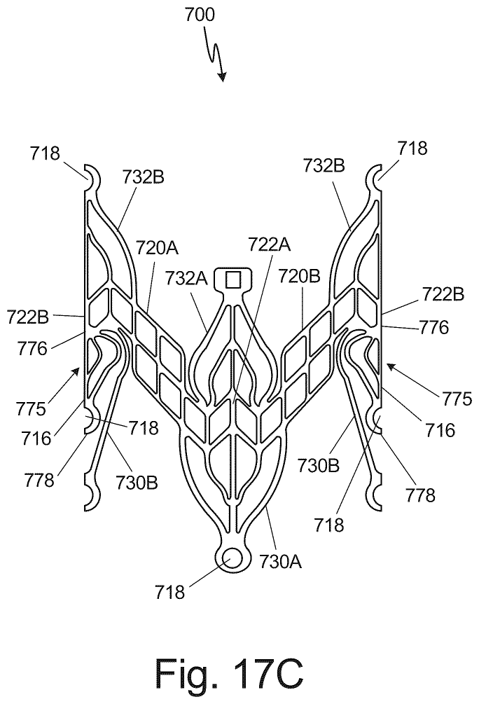

FIG. 17A is a side view of a first example of a shunt device including a secondary arm.

FIG. 17B is a top view of the first example of the shunt device including the secondary arm.

FIG. 17C is a flattened view of the first example of the shunt device including the secondary arm.

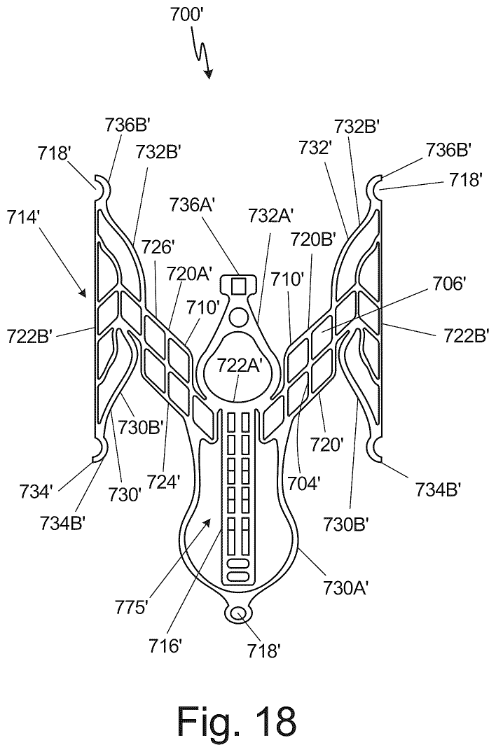

FIG. 18 is a flattened view of a second example of a shunt device including a secondary arm.

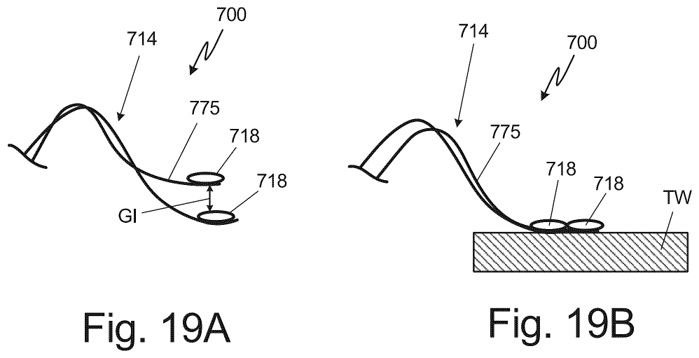

FIG. 19A is a schematic view of a shunt device including a secondary arm in a relaxed state.

FIG. 19B is a schematic view of the shunt device including the secondary arm in a deflected state.

### Shunt Devices 800, 800′, and 800″

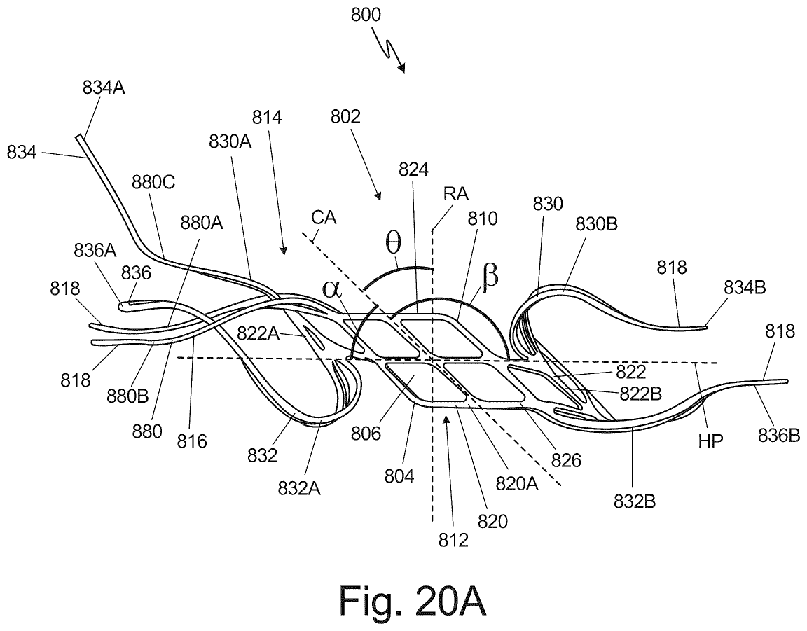

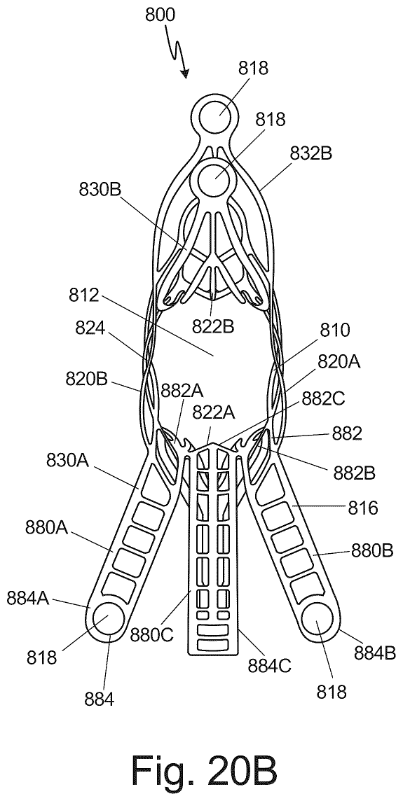

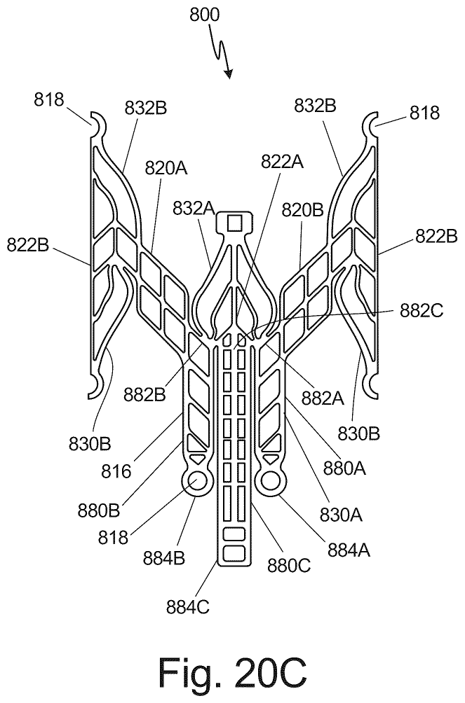

FIG. 20A is a side view of a first example of a shunt device including split arm portions and showing interlaced arms on a first side of the shunt device.

FIG. 20B is a top view of the first example of the shunt device including the split arm portions.

FIG. 20C is a flattened view of the first example of the shunt device including the split arm portions.

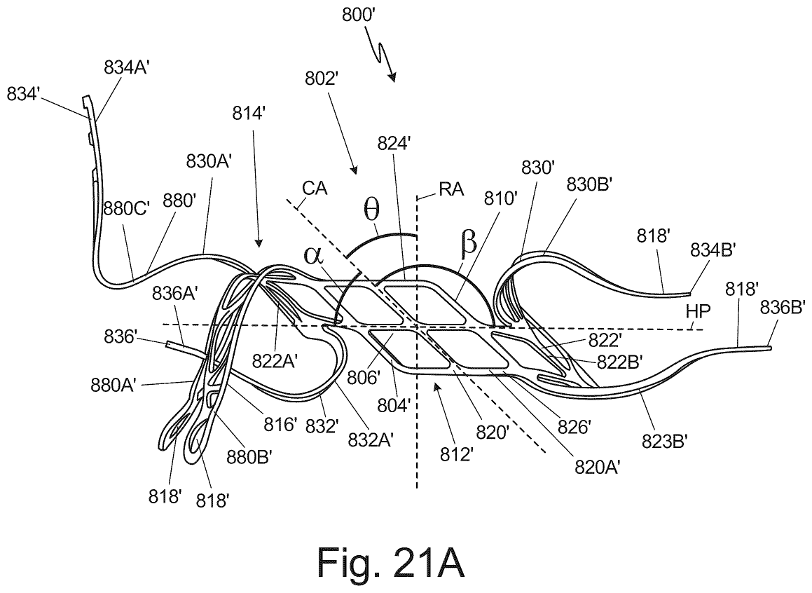

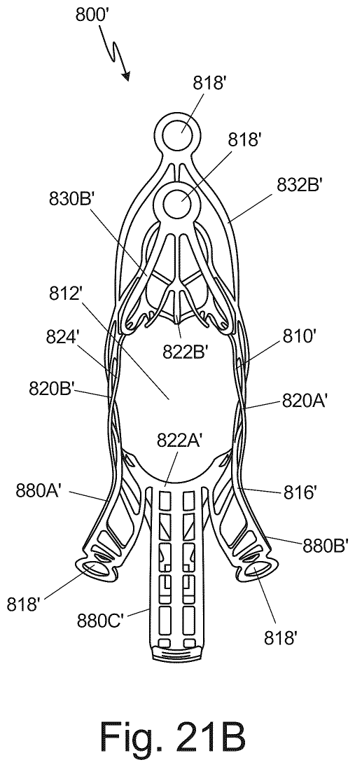

FIG. 21A is a side view of a second example of a shunt device including split arm portions and showing interlaced arms on a second side of the shunt device.

FIG. 21B is a top view of the second example of the shunt device including the split arm portions.

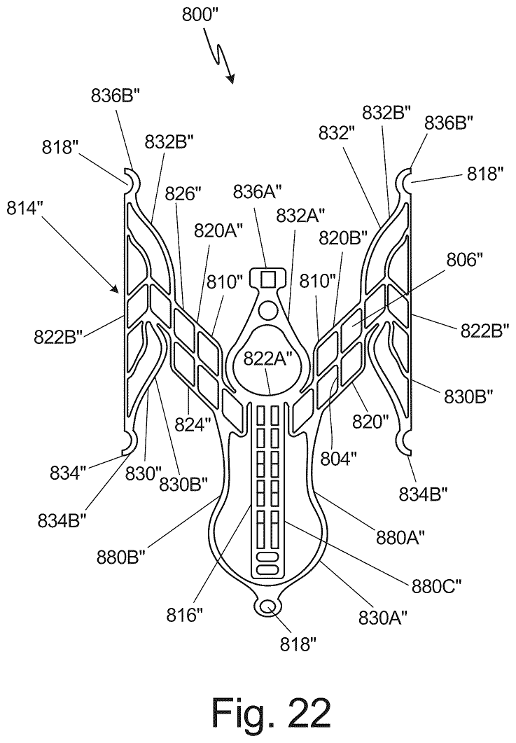

FIG. 22 is a flattened view of a third example of a shunt device including split arm portions.

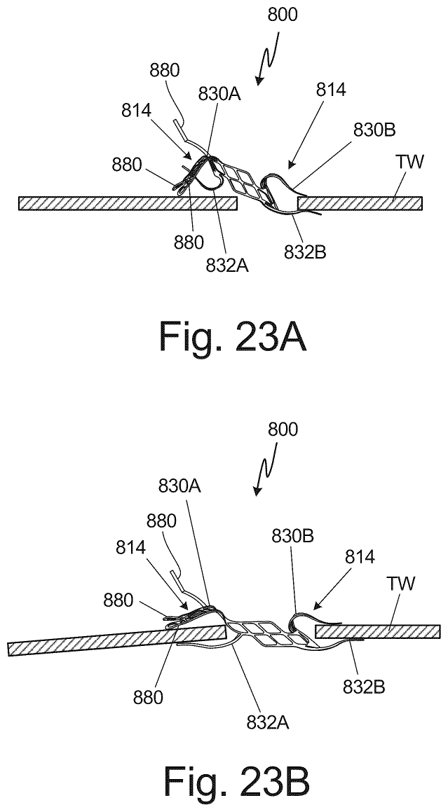

FIG. 23A is a schematic view of a shunt device including split arm portions in an interlaced state.

FIG. 23B is a schematic view of the shunt device including the split arm portions in a separated state.

### Shunt Devices 900 and 900′

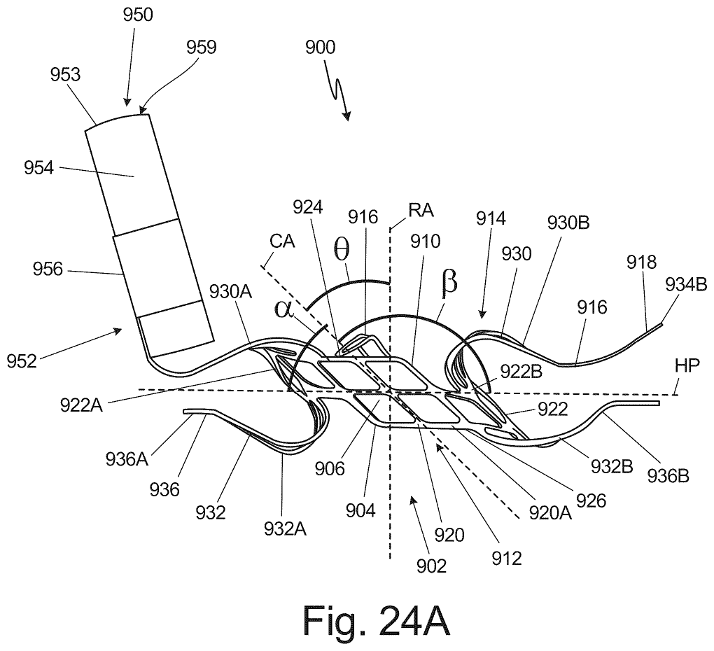

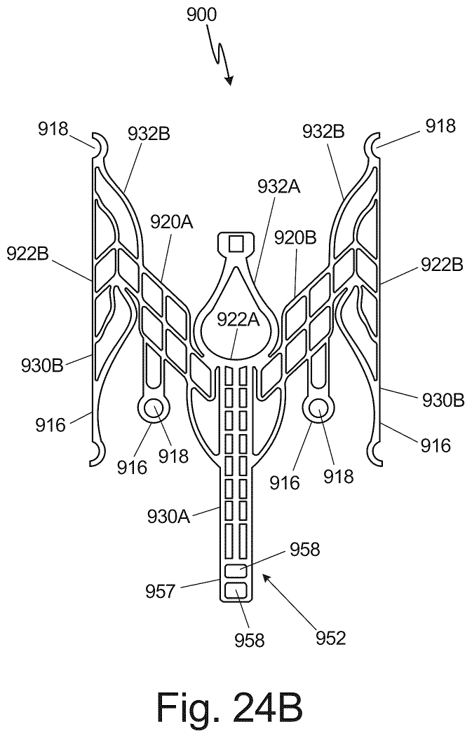

FIG. 24A is a side view of a first example of a shunt device including a sensor.

FIG. 24B is a flattened view of the first example of the shunt device with the sensor removed.

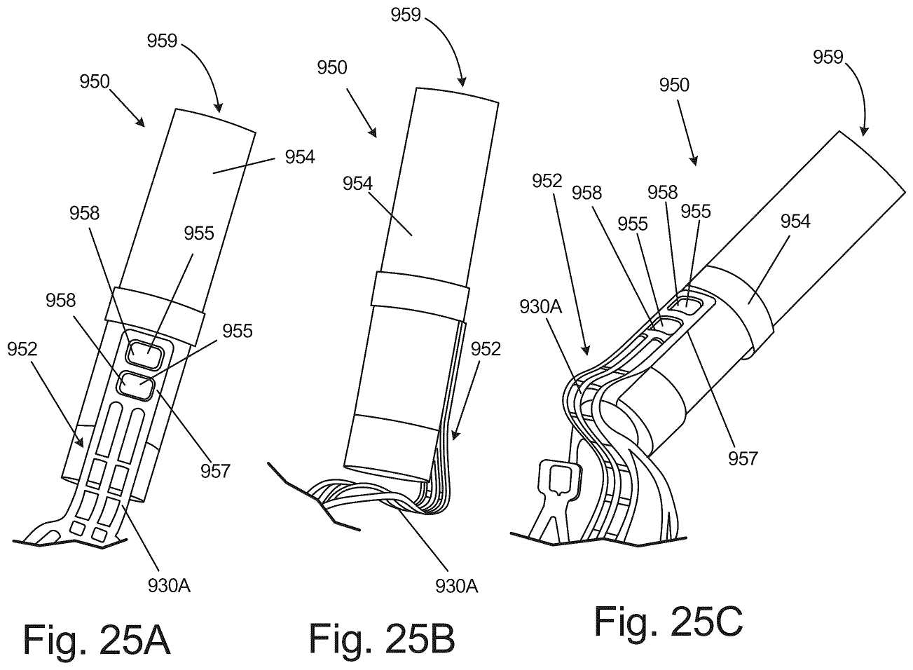

FIGS. 25A-25C are enlarged views showing details of the sensor and a sensor attachment portion of the shunt device.

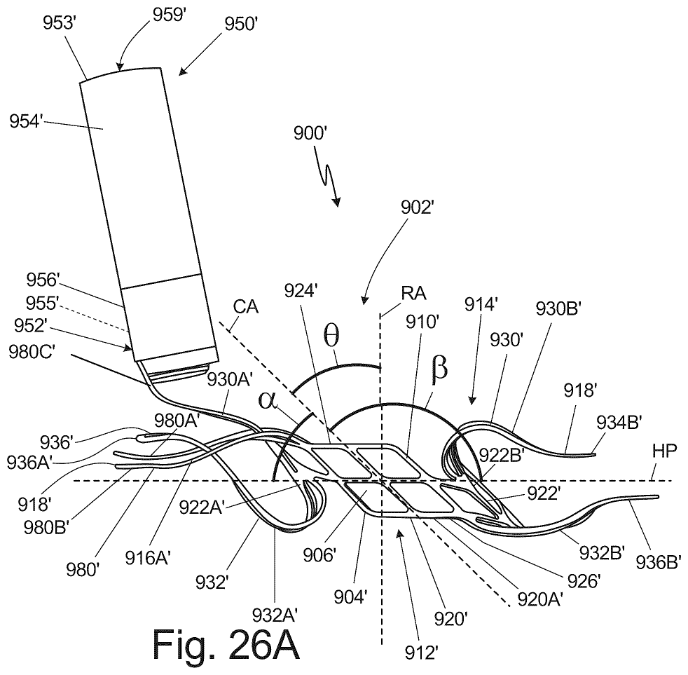

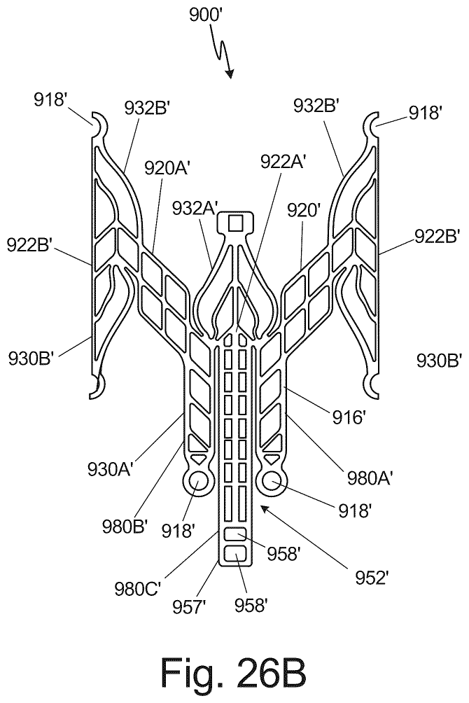

FIG. 26A is a side view of a second example of a shunt device including a sensor.

FIG. 26B is a flattened view of the second example of the shunt device with the sensor removed.

### DETAILED DESCRIPTION

### Anatomy of Heart H and Vasculature V (FIGS. 1-2)

FIG. 1 is a schematic diagram of heart H and vasculature V. FIG. 2 is a cross-sectional view of heart H. FIGS. 1-2 will be described together. FIGS. 1-2 show heart H, vasculature V, right atrium RA, right ventricle RV, left atrium LA, left ventricle LV, superior vena cava SVC, inferior vena cava IVC, tricuspid valve TV (shown in FIG. 1), pulmonary valve PV (shown in FIG. 1), pulmonary artery PA (shown in FIG. 1), pulmonary veins PVS, mitral valve MV, aortic valve AV (shown in FIG. 1), aorta AT (shown in FIG. 1), coronary sinus CS (shown in FIG. 2), thebesian valve BV (shown in FIG. 2), inter-atrial septum IS (shown in FIG. 2), and fossa ovalis FO (shown in FIG. 2).

Heart H is a human heart that receives blood from and delivers blood to vasculature V. Heart H includes four chambers: right atrium RA, right ventricle RV, left atrium LA, and left ventricle LV.

The right side of heart H, including right atrium RA and right ventricle RV, receives deoxygenated blood from vasculature V and pumps the blood to the lungs. Blood flows into right atrium RA from superior vena cava SVC and inferior vena cava IVC. Right atrium RA pumps the blood through tricuspid valve TV into right ventricle RV. The blood is then pumped by right ventricle RV through pulmonary valve PV into pulmonary artery PA. The blood flows from pulmonary artery PA into arteries that delivery the deoxygenated blood to the lungs via the pulmonary circulatory system. The lungs can then oxygenate the blood.

The left side of heart H, including left atrium LA and left ventricle LV, receives the oxygenated blood from the lungs and pumps the blood to the body. Blood flows into left atrium LA from pulmonary veins PVS. Left atrium LA pumps the blood through mitral valve MV into left ventricle LV. The blood is then pumped by left ventricle LV through aortic valve AV into aorta AT. The blood flows from aorta AT into arteries that deliver the oxygenated blood to the body via the systemic circulatory system.

Blood is additionally received in right atrium RA from coronary sinus CS. Coronary sinus CS collects deoxygenated blood from the heart muscle and delivers it to right atrium RA. Thebesian valve BV is a semicircular fold of tissue at the opening of coronary sinus CS in right atrium RA. Coronary sinus CS is wrapped around heart H and runs in part along and beneath the floor of left atrium LA right above mitral valve MV, as shown in FIG. 2. Coronary sinus CS has an increasing diameter as it connects to right atrium RA.

Inter-atrial septum IS and fossa ovalis FS are also shown in FIG. 2. Inter-atrial septum IS is the wall that separates right atrium RA from left atrium LA. Fossa ovalis FS is a depression in inter-atrial septum IS in right atrium RA. At birth, a congenital structure called a foramen ovale is positioned in inter-atrial septum IS. The foramen ovale is an opening in inter-atrial septum IS that closes shortly after birth to form fossa ovalis FS. The foramen ovale serves as a functional shunt in utero, allowing blood to move from right atrium RA to left atrium LA to then be circulated through the body. This is necessary in utero, as the lungs are in a sack of fluid and do not oxygenate the blood. Rather, oxygenated blood is received from the mother. The oxygenated blood from the mother flows from the placenta into inferior vena cava IVC through the umbilical vein and the ductus venosus. The oxygenated blood moves through inferior vena cava IVC to right atrium RA. The opening of inferior vena cava IVC in right atrium RA is positioned to direct the oxygenated blood through right atrium RA and the foramen ovale into left atrium LA. Left atrium LA can then pump the oxygenated blood into left ventricle LV, which pumps the oxygenated blood to aorta AT and the systemic circulatory system. This allows the pulmonary circulatory system to be bypassed in utero. Upon birth, respiration expands the lungs, blood begins to circulate through the lungs to be oxygenated, and the foramen ovale closes to form fossa ovalis FS.

Shunt devices can be positioned in heart H to shunt blood between left atrium LA and right atrium RA. Left atrium LA can experience elevated pressure due to abnormal heart conditions. It has been hypothesized that patients with elevated pressure in left atrium LA may benefit from a reduction of pressure in left atrium LA. Shunt devices can be used in these patients to shunt blood from left atrium LA to right atrium RA to reduce the pressure of blood in left atrium LA, which reduces the systolic preload on left ventricle LV. Reducing pressure in left atrium LA further relieves back-pressure on the pulmonary circulation to reduce the risk of pulmonary edema.

For example, shunt devices can be used to treat patients with heart failure (also known as congestive heart failure). The hearts of patients with heart failure do not pump blood as well as they should. Heart failure can affect the right side and/or the left side of the heart. Diastolic heart failure (also known as heart failure with preserved ejection fraction) refers to heart failure occurring when the left ventricle is stiff (having less compliance), which makes it hard to relax appropriately and fill with blood. This leads to increased end-diastolic pressure, which causes an elevation of pressure in left atrium LA. There are very few, if any, effective treatments available for diastolic heart failure. Other examples of abnormal heart conditions that cause elevated pressure in left atrium LA are systolic dysfunction of the left ventricle and valve disease.

Septal shunt devices (also called inter-atrial shunt devices) are positioned in inter-atrial septum IS to shunt blood directly from left atrium LA to right atrium RA. Typically, septal shunt devices are positioned in fossa ovalis FS, as fossa ovalis FS is a thinner area of tissue in inter-atrial septum IS where the two atria share a common wall. If the pressure in right atrium RA exceeds the pressure in left atrium LA, septal shunt devices can allow blood to flow from right atrium RA to left atrium LA. This causes a risk of paradoxical stroke (also known as paradoxical embolism), as emboli can move from right atrium RA to left atrium LA and then into aorta AT and the systemic circulation.

Shunt devices can also be left atrium to coronary sinus shunt devices that are positioned in a tissue wall between left atrium LA and coronary sinus CS where the two structures are in close approximation. Left atrium to coronary sinus shunt devices move blood from left atrium LA into coronary sinus CS, which then delivers the blood to right atrium RA via thebesian valve BV, the natural orifice of coronary sinus CS. Coronary sinus CS acts as an additional compliance chamber when using a left atrium to coronary sinus shunt device. Left atrium to coronary sinus shunt devices further provide increased protections against paradoxical strokes, as the blood would have to flow retrograde from right atrium RA through coronary sinus CS before entering left atrium LA. Further, left atrium to coronary sinus shunt devices also provide protection against significant right atrium RA to left atrium LA shunting, as again the blood would have to flow retrograde from right atrium RA through coronary sinus CS before entering left atrium LA.

### Shunt Devices 100 and 100′ (FIGS. 3A-5)

FIG. 3A is a perspective view of shunt device 100. FIG. 3B is a side view of shunt device 100. FIG. 4 is a perspective view of shunt device 100 in a collapsed configuration. FIGS. 3A, 3B, and 4 will be described together. Shunt device 100 includes body 102, which is formed of struts 104 and openings 106. Body 102 includes central flow tube 110, flow path 112, and arms 114. Shunt device 100 also includes tissue capture features 116. Central flow tube 110 has side portions 120 (including side portion 120A and side portion 120B), end portions 122 (including end portion 122A and end portion 122B), first axial end 124, and second axial end 126. Arms 114 include distal arms 130 (including distal arm 130A and distal arm 130B) and proximal arms 132 (including proximal arm 132A and proximal arm 132B). Distal arms 130 have terminal ends 134 (including terminal end 134A and terminal end 134B). Proximal arms 132 have terminal ends 136 (including terminal end 136A and terminal end 136B). FIG. 3B further shows gap G, horizontal reference plane HP, perpendicular reference axis RA, central axis CA, tilt angle θ, first angle α, and second angle β.

Shunt device 100 is a cardiovascular shunt. Shunt device 100 is shown in an expanded configuration in FIGS. 3A-3B. Shunt device 100 is formed of a super-elastic material that is capable of being compressed into a catheter for delivery into the body that can then retain its relaxed, or expanded, shape when it is released from the catheter. For example, shunt device 100 can be formed of a shape-memory material, such as nitinol (a nickel titanium alloy). Shunt device 100 is shown in a compressed configuration in FIG. 4. Upon delivery into the body, shunt device 100 will expand back to its relaxed, or expanded, shape. Shunt device 100 can be sterilized before being delivered into the body. Shunt device 100 has body 102 that is formed of interconnected struts 104. Openings 106 in body 102 are defined by struts 104. Body 102 of shunt device 100 is formed of struts 104 to increase the flexibility of shunt device 100 to enable it to be compressed and expanded.

Body 102 includes central flow tube 110 that forms a center portion of shunt device 100. Central flow tube 110 is tubular in cross-section but is formed of struts 104 and openings 106. Central flow tube 110 can be positioned in a puncture or opening in a tissue wall and hold the puncture open. Flow path 112 is an opening extending through central flow tube 110. Flow path 112 is the path through which blood flows through shunt device 100 when shunt device 100 is implanted in the body. Arms 114 extend from central flow tube 110. Arms 114 extend outward from central flow tube 110 when shunt device 100 is in an expanded configuration. Arms 114 hold shunt device 100 in position in the tissue wall when shunt device 100 is implanted in the body.

When shunt device 100 is implanted in the tissue wall between the left atrium and the coronary sinus of the heart, central flow tube 110 holds the puncture open so blood can flow from the left atrium to the coronary sinus through flow path 112. Struts 104 of central flow tube 110 form a lattice or cage of sorts that is sufficient to hold the puncture in the tissue wall open around central flow tube 110. Central flow tube 110 extends from first axial end 124 to second axial end 126. Central flow tube 110 is designed to have an axial length, as measured from first axial end 124 to second axial end 126, that approximates the thickness of the tissue wall between the left atrium and the coronary sinus. When shunt device 100 is implanted in the tissue wall between the left atrium and the coronary sinus, first axial end 124 can be facing the left atrium (i.e., a left atrial side of shunt device 100) and second axial end 126 can be facing the coronary sinus (i.e., a coronary sinus side of shunt device 100). In other examples, the orientation of first axial end 124 and second axial end 126 can be reversed.

Central flow tube 110 has side portions 120 and end portions 122. Side portion 120A and side portion 120B form opposing sides of central flow tube 110. End portion 122A and end portion 122B form opposing ends of central flow tube 110. End portion 122A and end portion 122B each extend between and connect to side portion 120A and side portion 120B to form a generally circular or oval opening that defines flow path 112. Side portions 120 and end portions 122 form a tubular lattice for central flow tube 110. Struts 104 of central flow tube 110 define openings 106 in central flow tube 110. In some examples, openings 106 can be generally parallelogram-shaped. In other examples, openings 106 can be any regular or irregular shape as desired. For example, struts 104 of side portions 120 can form an array of parallelogram-shaped openings 106 in side portions 120. Struts 104 of end portions 122 can form openings 106 in end portions 122. Struts 104 of arms 114 can form openings 106 in arms 114.

As shown in FIG. 3B, central flow tube 110 is angled with respect to horizontal reference plane HP extending through shunt device 100. Horizontal reference plane HP lies generally in the plane of the tissue wall immediately adjacent to shunt device 100 when shunt device 100 is implanted in the tissue wall. End portions 122 are similarly angled with respect to horizontal reference plane HP. Perpendicular reference axis RA, as shown in FIG. 3B, is perpendicular to horizontal reference plane HP. As shown in FIG. 3B, central axis CA is an axis through the center of central flow tube 110 and flow path 112. Central axis CA extends through central flow tube 110 at tilt angle θ with respect to perpendicular reference axis RA. Accordingly, central axis CA defines the angle or tilt of central flow tube 110 with respect to perpendicular reference axis RA (and horizontal reference plane HP). End portions 122 of central flow tube 110 extend parallel to central axis CA.

Arms 114 of shunt device 100 include two distal arms 130 and two proximal arms 132. In some examples, individual ones of distal arms 130 and/or proximal arms 132 can be formed of multiple split arm portions. Arms 114 extend outward from end portions 122 of central flow tube 110 when shunt device 100 is in an expanded configuration. Distal arm 130A is connected to and extends away from end portion 122A, and distal arm 130B is connected to and extends away from end portion 122B. Proximal arm 132A is connected to and extends away from end portion 122A, and proximal arm 132B is connected to and extends away from end portion 122B. When shunt device 100 is implanted in the tissue wall between the left atrium and the coronary sinus, distal arms 130 will be positioned in the left atrium and proximal arms 132 will be positioned in the coronary sinus. Distal arms 130 each have terminal ends 134. Specifically, distal arm 130A has terminal end 134A, and distal arm 130B has terminal end 134B. Proximal arms 132 each have terminal ends 136. Specifically, proximal arm 132A has terminal end 136A, and proximal arm 132B has terminal end 136B.

Distal arms 130 and proximal arms 132 curl outward from end walls 122. As shown in FIG. 3B, each of distal arms 130 and proximal arms 132 has a proximal portion adjacent to central flow tube 110 that forms a shallow curve or arc in a direction away from end walls 122 of central flow tube 110. Each of distal arms 130 and proximal arms 132 flattens out towards respective terminal ends 134 and 136 such that a portion of each of distal arms 130 and proximal arms 132 at or adjacent to the respective terminal end 134 or 136 is generally parallel to horizontal reference plane HP. Accordingly, an axis drawn through terminal end 134A and an axis drawn through terminal end 136B, which are approximated in FIG. 3B as axes in the plane of horizontal reference plane HP for simplicity, can each form first angle α with central axis CA through central flow tube 110. Similarly, an axis drawn through terminal end 134B, and an axis drawn through terminal end 136A, which are approximated in FIG. 3B as axes in the plane of horizontal reference plane HP for simplicity, can each form second angle β with central axis CA through central flow tube 110. Alternatively, distal arms 130 and proximal arms 132 do not flatten out and become parallel to horizontal reference plane HP but instead approach horizontal reference plane HP at an angle and/or have respective terminal ends 134 and 136 that angle away from horizontal reference plane HP. In such examples, first angle α and second angle β are approximations of the central angle for the arcs from end walls 122 to the tissue wall that each respective arm encompasses when shunt device 100 is implanted in the tissue wall. Put more simply, first angle α is the angle between central axis CA and horizontal reference plane HP, and second angle β is the supplementary angle to first angle α. In some examples, first angle α can be less than ninety degrees (<90°) and second angle β can be greater than ninety degrees (>90°). In other examples, first angle α and second angle β can be any suitable combination of angles that add to one hundred eighty degrees (180°). The difference between first angle α and second angle β and the corresponding curvature of ones of distal arms 130 and proximal arms 132) accommodates for the tilt of central flow tube 110.

As shown in FIG. 3B, distal arm 130A and distal arm 130B extend outwards from central flow tube 110 in opposite directions parallel to horizontal reference plane HP. Distal arm 130A and distal arm 130B can be aligned with each other (i.e., oriented at 180° to each other across central flow tube 110). In some examples, distal arm 130A has a longer length than distal arm 130B. In other examples, distal arm 130A has a shorter length than distal arm 130B. In yet other examples, distal arms 130 can have similar lengths. Proximal arm 132A and proximal arm 132B extend outwards from central flow tube 110 in opposite directions parallel to horizontal reference plane HP. Proximal arm 132A and proximal arm 132B can be aligned with each other (i.e., oriented at 180° to each other across central flow tube 110). In some examples, proximal arm 132A has a shorter length than proximal arm 132B. In other examples, proximal arm 132A has a longer length than proximal arm 132B. In yet other examples, proximal arms 132 can have similar lengths. In some examples, distal arm 130A has generally the same length and shape as proximal arm 132B, and distal arm 130B has generally the same length and shape as proximal arm 132A. In other examples, each of distal arms 130 and proximal arms 132 can have different lengths and shapes, though the overall shape of each arm is similar. As such, shunt device 100 has some degree of inverse symmetry across horizontal reference plane HP, as shown in FIG. 3B.

Shunt device 100 is generally elongated longitudinally but is relatively narrow laterally. Stated another way, distal arms 130 and proximal arms 132 are not annular or circular, but rather extend outward generally in only one plane. As shown in FIG. 3B, shunt device 100 has a generally H-shape when viewing a side of shunt device 100. The elongated shape of shunt device 100 means that when compressed it elongates along a line, as shown in FIG. 4, so as to better fit within a catheter.

Terminal ends 134 of distal arms 130 and terminal ends 136 of proximal arms 132 converge towards one another. Distal arms 130 and proximal arms 132 form two pairs of arms. That is, each of distal arms 130 forms a clamping pair with a corresponding one of proximal arms 132. Distal arm 130A and proximal arm 132A form a first pair of arms extending outward from a first side of central flow tube 110, and terminal end 134A of distal arm 130A converges towards terminal end 136A of proximal arm 132A. Distal arm 130B and proximal arm 132B form a second pair of arms extending outward from a second side of central flow tube 110, and terminal end 134B of distal arm 130B converges towards terminal end 136B of proximal arm 132B. Gap G between terminal ends 134 and terminal ends 136 is sized to be slightly smaller than an approximate thickness of the tissue wall between the left atrium and the coronary sinus, or another tissue wall of interest. This allows distal arms 130 and proximal arms 132 to flex outwards and grip the tissue wall when implanted to help hold shunt device 100 in place against the tissue wall. Thus, a distance corresponding to gap G, as measured once shunt device 100 is implanted, may be slightly different between different clamping pairs of distal arms 130 and proximal arms 132 depending on anatomical variations along the particular tissue wall. Terminal ends 134 of distal arms 130 and terminal ends 136 of proximal arms 132 can also have openings or indentations that are configured to engage a delivery tool to facilitate implantation of shunt device 100, for example actuating rods of a delivery tool. Additionally, terminal ends 134 of distal arms 130 and terminal ends of proximal arms 132 can include locations for radiopaque markers to permit visualization of the positioning of shunt device 100.

When implanted in the tissue wall, distal arms 130 and proximal arms 132 are designed such that the projection of distal arms 130 and proximal arms 132 into the left atrium and the coronary sinus, respectively, is minimized. This minimizes the disruption of the natural flow patterns in the left atrium and the coronary sinus. Shunt device 100 can also be designed so that the profile of proximal arms 132 projecting into the coronary sinus is lower than the profile of distal arms 130 projecting into the left atrium to minimize disruption of the natural blood flow through the coronary sinus and to reduce the potential for proximal arms 132 to block the narrower passage of the coronary sinus.

Tissue capture features 116 can take several different forms. For example, tissue capture features 116 connected to central flow tube 110 at first axial end 124 and/or second axial end 126 can be tabs that extend outward from side portions 120. Tissue capture features 116 connected to arms 114 can be deflectable projections that extend between respective ones of arms 114 and the tissue wall to be compressed back toward the respective arm 114 when shunt device 100 is implanted in the tissue wall. Tissue capture features 116 connected to end portions 122 of central flow tube 110 can be secondary arms associated with one of arms 114. Tissue capture features 116 that are a part of arms 114 themselves can be, e.g., a lengthened portion of one of arms 114, separate split arm portions of one of arms 114, and/or interlacing arms 114. Any one or more of tissue capture features 116 can be incorporated alone or in combination on shunt device 100 to aid in anchoring shunt device 100 to the tissue wall and to prevent displacement of shunt device 100.

FIG. 5 is a perspective view of shunt device 100′ including sensor 150′. Shunt device 100′ includes body 102′, which is formed of struts 104′ and openings 106′. Body 102′ includes central flow tube 110′, flow path 112′, arms 114′. Shunt device 100′ also includes and tissue capture features 116′. Central flow tube 110′ has side portions 120′ (including side portion 120A′ and side portion 120B′), end portions 122′ (including end portion 122A′ and end portion 122B′), first axial end 124′, and second axial end 126′. Arms 114′ include distal arms 130′ (including distal arm 130A′ and distal arm 130B′) and proximal arms 132′ (including proximal arm 132A′ and proximal arm 132B′). Distal arms 130′ have terminal ends 134′ (including terminal end 134A′ and terminal end 134B′). Proximal arms 132′ have terminal ends 136′ (including terminal end 136A′ and terminal end 136B′). Shunt device 100′ further includes sensor 150′ and sensor attachment portion 152′.

Shunt device 100′ includes a similar structure and design to shunt device 100 described above, except shunt device 100′ additionally includes sensor 150′ connected to sensor attachment portion 152′.

As shown in FIG. 5, sensor 150′ can be attached to shunt device 100′ so that sensor 150′ is positioned in the left atrium when shunt device 100′ is implanted in the tissue wall between the left atrium and the coronary sinus of the heart. Accordingly, sensor 150′ can be attached to one of distal arms 130′. Alternatively, sensor 150′ can be attached to shunt device 100′ so that sensor 150′ is positioned in the coronary sinus when shunt device 100′ is implanted in the tissue wall. In such examples, sensor 150′ can be attached to one of proximal arms 132′. In further examples, an additional sensor can be included on shunt device 100′ to position sensors in both the left atrium and the coronary sinus.

Sensor 150′ is attached to shunt device 100′ at sensor attachment portion 152′. Sensor 150′ can be connected to sensor attachment portion 152′ using any suitable attachment mechanism. For example, sensor 150′ and sensor attachment portion 152′ can include complimentary mating features. Sensor attachment portion 152′ can be an extension of one of arms 114′ of shunt device 100′. In some examples, sensor attachment portion 152′ is an extension of distal arm 130A′. In other examples, sensor attachment portion 152′ is an extension of distal arm 130B′ or one of proximal arms 132′. Alternatively, as shown in FIG. 5, sensor attachment portion 152′ can be a separate split arm portion of one of arms 114′. Sensor attachment portion 152′ can be angled away from a horizontal reference plane (not shown) that is in the plane of the tissue wall adjacent to shunt device 100′ when shunt device 100′ is implanted in the tissue wall. That is, sensor attachment portion 152′ can be angled away from the tissue wall.

Sensor 150′ can be a pressure sensor to sense a pressure in the left atrium. In other examples, sensor 150′ can be any sensor to measure a parameter in the left atrium. In yet other examples, sensor 150′ can be any sensor to measure a parameter in the coronary sinus. Sensor 150′ can include a transducer, control circuitry, and an antenna in one example. The transducer, for example a pressure transducer, is configured to sense a signal from the left atrium. The transducer can communicate the signal to the control circuitry. The control circuitry can process the signal from the transducer or communicate the signal from the transducer to a remote device outside of the body using the antenna. Sensor 150′ can include alternate or additional components in other examples. Further, the components of sensor 150′ can be held in a sensor housing that is hermetically sealed.

### Delivery Catheter 200 (FIGS. 6-7B)

FIG. 6 is a side view of delivery catheter 200. FIG. 7A is a side view of distal portion 214 of delivery catheter 200 in a sheathed state. FIG. 7B is a side view of distal portion 214 of delivery catheter 200 in an unsheathed state. FIGS. 6, 7A, and 7B will be discussed together. FIGS. 6-7B show delivery catheter 200. FIG. 7B shows shunt device 202. Delivery catheter 200 includes proximal end 200A, distal end 200B, proximal portion 210, intermediate portion 212, distal portion 214, handle 216, outer sheath 218, inner sheath 220, bridge 222, nosecone 224, actuation rod 226, side opening 228, and notch 229.

Delivery catheter 200 is one example of a delivery catheter that can be used to implant a shunt device into a patient. Delivery catheter 200 as shown in FIGS. 6-7B is used to implant shunt device 202 (shown in FIG. 7B). Delivery catheter 200 can take other forms in alternate examples. Shunt device 202 can have the structure and design of any suitable shunt device, for example shunt device 100 or 100′ as shown in FIGS. 3A-5. Delivery catheter 200 is shown as being configured to implant shunt device 202 without a sensor in the example shown in FIGS. 6-7B. In alternate examples, delivery catheter 200 can be used to implant a shunt device with a sensor, including any needed modifications to accommodate the sensor.

Delivery catheter 200 includes proximal portion 210 adjacent proximal end 200A of delivery catheter 200, intermediate portion 212 extending from proximal portion 210, and distal portion 214 extending from intermediate portion 212 to distal end 200B of delivery catheter 200. Proximal portion 210 includes handle 216, which can be grasped by a physician to control movement of delivery catheter 200. Handle 216 includes a number of ports through which guide wires, tubes, fluids, or other components or elements may be passed.

Intermediate portion 212 extends outward from handle 216 and is a length of catheter that can be moved through a patient. Outer sheath 218 and inner sheath 220 extend outward from handle 216 and form a portion of intermediate portion 212. Outer sheath 218 covers inner sheath 220.

Distal portion 214 extends from intermediate portion 212. Distal portion 214 includes bridge 222 and nosecone 224. Bridge 222 extends from inner sheath 220 towards nosecone 224. Nosecone 224 extends from bridge 222 to distal end 200B of delivery catheter 200. Bridge 222 is configured to hold shunt device 202. As shown in FIG. 7A, when delivery catheter 200 is in a sheathed state, outer sheath 218 will extend over and cover shunt device 202 on bridge 222. As shown in FIG. 7B, when delivery catheter 200 is in an unsheathed state, outer sheath 218 will be pulled back to expose bridge 222 and shunt device 202 on bridge 222. Nosecone 224 extends outward from bridge 222 and helps guide delivery catheter 200 through a patient's vasculature. Actuation rod 226, also called an actuation arm, extends through a lumen in inner sheath 220 and bridge 222. Actuation rod 226 emerges from side opening 228 in bridge 222 and connects to a first proximal arm of shunt device 202. Side opening 228 extends into a body of bridge 222. Notch 229 extends into the body of bridge 222 opposite side opening 228. Notch 229 is configured to seat a second proximal arm of shunt device 202. The second proximal arm can be retained on bridge 222 prior to deployment by a release wire (not shown) extending through a lumen of bridge 222 and through notch 229.

Delivery catheter 200 will be discussed below in more detail with respect to FIGS. 8A-9R.

### Delivery Method 300 (FIGS. 8A-9R)

FIG. 8A is a flow chart showing steps for creating a puncture in tissue wall TW between coronary sinus CS and left atrium LA. FIG. 8B is a flow chart showing steps for implanting shunt device 202 in tissue wall TW between coronary sinus CS and left atrium LA. FIGS. 9A-9R are schematic views showing the steps for implanting shunt device 202 in tissue wall TW between coronary sinus CS and left atrium LA. FIGS. 8A-9R will be discussed together. FIGS. 8A-8B show method 300. FIG. 8A shows steps 302-316 of method 300. FIG. 8B shows steps 318-334 of method 300.

Step 302 includes advancing guidewire 230 into coronary sinus CS, as shown in FIG. 9A. Guidewire 230 can be inserted using traditional methods. Guidewire 230 is inserted into right atrium RA, through an ostium of coronary sinus CS, and then into coronary sinus CS. Optionally, a catheter having radiopaque markers can be inserted over guidewire 230 and imaging can be done to confirm placement of guidewire 230 in coronary sinus CS. Additionally, contrast can be injected into coronary sinus CS through the catheter to further confirm placement of guidewire 230 in coronary sinus CS. The catheter can then be removed once placement of guidewire 230 in coronary sinus CS is confirmed.

Step 304 includes advancing puncture catheter 232 over guidewire 230 to coronary sinus CS, as shown in FIG. 9B. Puncture catheter 232 is used to puncture tissue wall TW between coronary sinus CS and left atrium LA. Puncture catheter 232 includes catheter body 234 having opening 236 on a first side and balloon 238 on a second side opposite opening 236. Puncture catheter 232 can also include radiopaque markers 239 proximal and distal to opening 236 to confirm placement of puncture catheter 232 in coronary sinus CS. Puncture catheter 232 is advanced into coronary sinus CS so that opening 236 is facing tissue wall TW between coronary sinus CS and left atrium LA. Puncture catheter 232 shown in FIG. 9B is one example of a puncture catheter. In alternate examples, tissue wall TW can be punctured using other puncture catheters or other suitable mechanisms.

Step 306 includes inflating balloon 238 of puncture catheter 232, as shown in FIG. 9C. As balloon 238 is inflated, it will press against coronary sinus CS opposite of tissue wall TW. The inflation of balloon 238 will press puncture catheter 232 against tissue wall TW. Specifically, opening 236 will be pressed against tissue wall TW. Balloon 238 will anchor puncture catheter 232 in position in coronary sinus CS while a puncture is made in tissue wall TW. In alternate examples, any other suitable anchoring mechanism can be used instead of balloon 238. In further examples, step 306 is not needed.

Step 308 includes puncturing tissue wall TW between coronary sinus CS and left atrium LA, as shown in FIG. 9D. Puncture catheter 232 includes puncture arm 240 extending through a lumen in puncture catheter 232. Puncture arm 240 includes sheath 242 and needle 244 positioned in sheath 242 so that it extends out a distal end of puncture sheath 242. Puncture arm 240 can be advanced through puncture catheter 232 and out of opening 236 to puncture through tissue wall TW between coronary sinus CS and left atrium LA.

Puncture catheter 232 should be positioned in coronary sinus CS so that opening 236 of puncture catheter 232 is positioned 2-4 centimeters from the ostium of coronary sinus CS. This will position the puncture through tissue wall TW at the same location. The puncture, and ultimately the placement of shunt device 202 in the puncture, is positioned over the posterior leaflet of mitral valve MV.

Step 310 includes removing needle 244 from puncture catheter 232, as shown in FIG. 9E. Needle 244 can be removed by pulling it proximally through a lumen extending through needle sheath 242 of puncture arm 240. Needle 244 is fully removed from puncture catheter 232, leaving a lumen extending from a proximal end of puncture catheter 232 through a distal end of needle sheath 242.

Step 312 includes advancing guidewire 246 through puncture catheter 232 into left atrium LA, as shown in FIG. 9F. Specifically, guidewire 246 is advanced through a lumen extending through a proximal end of puncture catheter 232 and needle sheath 242 of puncture arm 240. Guidewire 246 is advanced into left atrium LA until it coils in left atrium LA, as shown in FIG. 9F. Once guidewire 246 is fully positioned in left atrium LA, puncture catheter 232 and guidewire 230 can be removed from left atrium LA and coronary sinus CS.

Step 314 includes advancing balloon catheter 248 over guidewire 246 and through the puncture in tissue wall TW, as shown in FIG. 9G. Balloon catheter 248 is advanced through the puncture in tissue wall TW so balloon 250 of balloon catheter 248 is positioned in the puncture in tissue wall TW. Balloon catheter 248 is shown as being a separate device from puncture catheter 232 in the example shown in FIG. 9G. However, in alternate examples, balloon catheter 248 can be inserted through puncture catheter 232 and through the puncture in tissue wall TW.

Step 316 includes inflating balloon 250 of balloon catheter 248 extending through the puncture in tissue wall TW, as shown in FIG. 9H. Balloon 250 extends along a distal portion of balloon catheter 248. As balloon 250 is inflated, it will expand and push open the tissue surrounding the puncture in tissue wall TW. The inflation of balloon 250 will cause the puncture in tissue wall TW to become a wider opening in which a shunt device can be positioned. Balloon 250 can then be deflated and balloon catheter 248 can be removed from left atrium LA and coronary sinus CS.

Step 318 includes advancing delivery catheter 200 over guidewire 246, as shown in FIG. 9I. Delivery catheter 200 has the general structure and design as discussed with reference to FIGS. 6-7B above. Delivery catheter 200 is inserted through coronary sinus CS, through the opening in tissue wall TW, and into left atrium LA. When delivery catheter 200 is properly positioned in tissue wall TW, nosecone 224 will be positioned in left atrium LA, and bridge 222 will extend through tissue wall TW between left atrium LA and coronary sinus CS. Nosecone 224 tapers from a smaller diameter at a distal end to a larger diameter at a proximal end. The taper of nosecone 224 helps to advance nosecone 224 through the opening in tissue wall TW and widens the opening as needed. Bridge 222 holds shunt device 202 (not shown in FIG. 9I) in a collapsed position on bridge 222. Bridge 222 is positioned in tissue wall TW so that shunt device 202 is generally positioned in the opening in tissue wall TW for deployment into the opening.

Step 320 includes withdrawing outer sheath 218 of delivery catheter 200 to release distal arms 252 of shunt device 202, as shown in FIG. 9J. Outer sheath 218 can be withdrawn to expose part of shunt device 202 held on bridge 222 of delivery catheter 200. As outer sheath 218 is withdrawn, distal arms 252 of shunt device 202 will be released and assume their preset shape. Delivery catheter 200 should be positioned in left atrium LA such that when outer sheath 218 is withdrawn to release distal arms 252 of shunts device 202, distal arms 252 of shunt device 202 are positioned in left atrium LA.

Step 322 includes pulling delivery catheter 200 proximally to seat distal arms 252 of shunt device 202 on tissue wall TW, as shown in FIG. 9K. Delivery catheter 200 can be gently pulled proximally to seat distal arms 252 of shunt device 202 on tissue wall TW in left atrium LA. A physician should stop gently pulling on delivery catheter 200 when resistance is sensed, indicating that distal arms 252 have come into contact with tissue wall TW. This will also position a central flow tube of shunt device 202 in the opening in tissue wall TW.

Step 324 includes withdrawing outer sheath 218 of delivery catheter 200 to expose proximal arms 254 of shunt device 202, as shown in FIG. 9L. Outer sheath 218 is withdrawn a set distance to fully expose shunt device 202, including proximal arms 254 of shunt device 202. Delivery catheter 200 should be positioned in left atrium LA, tissue wall TW, and coronary sinus CS so that proximal arms 254 will be positioned in coronary sinus CS when outer sheath 218 is withdrawn. Proximal arms 254 are constrained on bridge 222 of delivery catheter 200 and will not automatically assume their preset shape when outer sheath 218 is withdrawn.

Step 326 includes moving first proximal arm 254A of shunt device 202 towards tissue wall TW using actuation rod 226 of delivery catheter 200, as shown in FIG. 9M. Actuation rod 226 extends through a lumen in delivery catheter 200 and can be actuated forward to move first proximal arm 254A towards tissue wall TW.

Step 328 includes seating first proximal arm 254A on tissue wall TW, as shown in FIG. 9N. Actuation rod 226 of delivery catheter 200 is actuated fully outward to seat first proximal arm 254A on tissue wall TW. When first proximal arm 256A is seated on tissue wall TW, it will be positioned in coronary sinus CS.

Step 330 includes injecting contrast into coronary sinus CS and left atrium LA to confirm placement of shunt device 202 in tissue wall TW, as shown in FIG. 9O. Contrast can be injected through a lumen extending through delivery catheter 200. The contrast can move through coronary sinus CS and left atrium LA. The contrast will highlight shunt device 202 under fluoroscopy to confirm proper placement of distal arms 252 and first proximal arm 254A of shunt device 202 on tissue wall TW.

Step 332 includes removing actuation rod 226 from first proximal arm 254A of shunt device 202, as shown in FIG. 9P. Actuation rod 226 can be held on and removed from first proximal arm 254A using any suitable mechanism. In the example shown in FIG. 9P, a release wire holds actuation rod 226 on first proximal arm 254A. The release wire can be withdrawn proximally to disconnect release wire from first proximal arm 254A. Actuation rod 226 can then be pulled proximally through a lumen of delivery catheter 200 to remove actuation rod 226 from coronary sinus CS.

Step 334 includes withdrawing delivery catheter 200 from coronary sinus CS and left atrium LA to release second proximal arm 254B of shunt device 202, as shown in FIG. 9Q. Second proximal arm 254B is held in place on bridge 222 in notch 229 formed in bridge 222. As delivery catheter 200 is withdrawn, second proximal arm 254B will be released from notch 229 in bridge 222 and take its preset shape. Specifically, second proximal arm 254B will seat upon tissue wall TW as it takes its preset shape. Second proximal arm 245B will be positioned in coronary sinus CS. After second proximal arm 254B is seated on tissue wall TW, shunt device 202 will be fully deployed in tissue wall TW, as shown in FIG. 9R. Delivery catheter 200 and guidewire 246 can then be removed from left atrium LA and coronary sinus CS.

Method 300 is one example of a method that can be used to implant shunt device 202 in tissue wall TW between left atrium LA and coronary sinus CS. Method 300 can include fewer, more, or different steps in alternate examples. Further, puncture catheter 232 and delivery catheter 200 are shown as being separate catheters in the example shown in FIGS. 9A-9R, but can be a single catheter in alternate examples.

Improper positioning or mis-seating of a shunt device can place a patient at risk. In practice, shunt devices must be anchored in place to avoid displacement during normal heart rhythms. FIG. 9R shows shunt device 202 properly seated in tissue wall TW between left atrium LA and coronary sinus CS. As illustrated, distal arms 252 engage walls of left atrium LA and proximal arms 254 engage adjacent walls of coronary sinus CS. During deployment, one or more distal arms 252 or proximal arms 254 can be mis-seated. In one example, shunt device 202 could be improperly seated such that one or more of distal arms 252 is positioned in coronary sinus CS rather than in left atrium LA. For example, during an implantation procedure (e.g., during step 322 of method 300 as shown in FIG. 8B), a physician may pull delivery catheter 200 back too hard after distal arms 252 are released, causing all or a part of shunt device 202 to be pulled into coronary sinus CS. In another example, shunt device 202 could be improperly seated such that the entirety of shunt device 202 is located in left atrium LA. For example, during an implantation procedure (e.g., during step 322 of method 300 as shown in FIG. 8B), a physician may not pull the delivery catheter back far enough after distal arms 252 are released, so one or more of proximal arms 254 may be released in or pushed through to left atrium LA, causing shunt device 202 to embolize. Confirming tissue capture between the arms of a shunt device helps a physician to determine when it is safe to release the shunt device. As such, confirming proper seating of the shunt device during and/or following delivery also helps reduce the risk of embolization and/or need for redeployment. The present disclosure includes several shunt device features for confirming tissue capture and ensuring proper placement of a shunt device. These features can be included on shunt devices with or without sensors attached.

Several examples of features of shunt devices according to techniques of this disclosure will be described with reference to FIGS. 10A-26B. Each shunt device example shown in FIGS. 10A-26B includes generally similar components, which are identified by shared reference numbers that are increased incrementally between sets of FIGS. 10A-10C, 11A-12B, 13A-16B, 17A-19B, 20A-23B, and 24A-26B (e.g., FIGS. 10A-10C include shunt device 400; FIGS. 11A-12B include shunt devices 500 and 500′; FIGS. 13A-16B include shunt devices 600, 600′, and 600″; FIGS. 17A-19B include shunt devices 700 and 700′; FIGS. 20A-23B include shunt devices 800, 800′, and 800″; and FIGS. 24A-26B include shunt devices 900 and 900′). Further, each shunt device example shown in FIGS. 10A-26B can be an example of shunt devices 100 and 100′ (described above in reference to FIGS. 3A-5), with similar components sharing the same name. For ease of discussion, some components of the shunt device examples shown in FIGS. 10A-26B are not described in detail in the following sections, but it should be understood that the shunt device examples shown in FIGS. 10A-26B can include all or any combination of the components and features described above with respect to FIGS. 3A-5. Additionally, although depicted in FIGS. 10A-26B as separate examples, a shunt device according to techniques of this disclosure can include any combination of the following features.

### Shunt Device 400 (FIGS. 10A-10C)

FIG. 10A is a perspective view of shunt device 400 including lengthened portion 460. FIG. 10B is a side view of shunt device 400 including lengthened portion 460. FIG. 10C is a flattened view of shunt device 400 including lengthened portion 460. FIGS. 10A-10C will be described together. As illustrated in FIGS. 10A-10C, shunt device 400 includes body 402, which is formed of struts 404 and openings 406. Body 402 includes central flow tube 410, flow path 412, and arms 414. Shunt device 400 also includes tissue capture features 416 and radiopaque markers 418. Central flow tube 410 has side portions 420 (including side portion 420A and side portion 420B), end portions 422 (including end portion 422A and end portion 422B), first axial end 424, and second axial end 426. Arms 414 include distal arms 430 (including distal arm 430A and distal arm 430B) and proximal arms 432 (including proximal arm 432A and proximal arm 432B). Distal arms 430 have terminal ends 434 (including terminal end 434A and terminal end 434B). Proximal arms 432 have terminal ends 436 (including terminal end 436A and terminal end 436B). As is further illustrated in FIGS. 10A-10C, tissue capture features 416 include lengthened portion 460, which is further divided into first portion 462 and second portion 464. FIG. 10B further shows horizontal reference plane HP, perpendicular reference axis RA, central axis CA, tilt angle θ, first angle α, and second angle β. FIG. 10C shows a flattened view of shunt device 400 as though shunt device 400 has been separated or cut along a cut line (not shown) drawn longitudinally through distal arm 430B, through end portion 422B, and longitudinally through proximal arm 432B.

Shunt device 400 has a generally similar structure and design to shunt devices 100 and 100′ described above in reference to FIGS. 3A-5, and shunt device 400 includes lengthened portion 460. Lengthened portion 460 is described here with respect to shunt device 400 but can also be included on any of the examples of a shunt device descried herewith, including shunt devices 100, 100′, 500, 500′, 600, 600′, 600″, 700, 700′, 800, 800′, 800″, 900, and 900′.

Lengthened portion 460 is one form of tissue capture features 416. Lengthened portion 460 is a structural region along the length of at least one of arms 414. More specifically, lengthened portion 460 is an elongated portion of at least one of arms 414 situated between a respective one of terminal ends 434, 436 and a respective proximal portion that is adjacent to central flow tube 410. The one or more of arms 414 that include lengthened portion 460 will be referred to herein as lengthened arm 414. In some examples, lengthened portion 460 can be part of one of distal arms 430. As illustrated in FIGS. 10A-10C, lengthened portion 460 can be part of distal arm 430B. Accordingly, lengthened portion 460 can be between a proximal portion of distal arm 430B and terminal end 434B. In other examples, lengthened portion 460 can be part of one of proximal arms 432. In yet other examples, multiple ones of arms 414 can include respective lengthened portions 460. Generally, any, some, or all of arms 414 can include lengthened portion 460.

Including lengthened portion 460 as part of one of arms 414 increases the overall length of that arm (as measured from central flow tube 410 to the respective terminal end 434, 436) compared to an equivalent arm without lengthened portion 460. Moreover, the lengthened arm that includes lengthened portion 460 can be longer than a corresponding arm 414 with which it forms a clamping pair. For example, as illustrated in FIGS. 10A-10C, distal arm 430B including lengthened portion 460 is longer than corresponding proximal arm 432B.

As is most easily seen in FIG. 10B, lengthened portion 460 extends generally parallel to horizontal reference plane HP. Lengthened portion 460 is also generally straight and is narrower than a respective proximal portion of the lengthened arm 414. In some examples, several struts 404 in the respective proximal portion of the lengthened arm 414 converge and merge into lengthened portion 460. In some examples, struts 404 adjacent to lengthened portion 460 are thicker or more rigid to accommodate and support deflection of the lengthened arm 414.

Lengthened portion 460 includes first portion 462 and second portion 464. First portion 462 is a first structural region of lengthened portion 460 that is adjacent to and extends from the respective proximal portion of the lengthened arm 414. First portion 462 can be tapered such that it is widest towards central flow tube 410 and narrowest towards the respective terminal end 434, 436. Second portion 464 is a second structural region of lengthened portion 460 that is between first portion 462 and the respective terminal end 434, 436 of the lengthened arm 414. Second portion 464 can include the narrowest region of lengthened portion 460.

In general, the physical dimensions of lengthened portion 460 can be selected to avoid interaction between distal arm 430B (or the ones of arms 414 include lengthened portion 460) and nearby anatomical features when shunt device 400 is implanted in a puncture in a tissue wall. For example, lengthened portion 460 can be sized to avoid interaction with a mitral valve in a left atrium of a heart. The physical dimensions of lengthened portion 460 can also be selected based on a desired amount of deflection of the one of arms 414 that includes lengthened portion 460 and balanced against fatigue and strain considerations. The desired amount of deflection can be calibrated for optimal visualization during implantation of shunt device 400.

Radiopaque markers 418 can be included on shunt device 400. Radiopaque markers 418 are structures that are dense and resist the passage of X-rays to permit visualization with radiographic imaging. For example, radiopaque marker 418 can be attached or stamped at a respective terminal end 434, 436, of the one of arms 414 that includes lengthened portion 460, e.g., terminal end 434B of distal arm 430B. In some examples, radiopaque markers 418 can be attached at multiple ones of terminal ends 434, 436 and to other tissue capture features 416. For example, FIGS. 10A-10C show radiopaque markers 418 at terminal ends 434A, 434B, and 436B and associated with ones of tissue capture features 416. In other examples, radiopaque markers 418 can be attached to any suitable portion or portions of shunt device 400, such as other locations on arms 414 or other locations on tissue capture features 416. Moreover, shunt device 400 can include any suitable number of radiopaque markers 418. In yet other examples, shunt device 400 may not include any radiopaque markers 418. Radiopaque markers 418 can be shaped and sized to fit on the portions of shunt device 400 to which they are attached. In other examples, radiopaque markers 418 can be thickened regions of struts 404 rather than separate pieces that are attached. For example, shunt device 400 can be formed of nitinol (a nickel titanium alloy) and radiopaque markers 418 can be relatively thickened regions of nitinol.

Shunt device 400 can be delivered into a human body using, for example, delivery catheter 200 (as shown in FIGS. 6-7B) and according to, for example, method 300 described above in reference to FIGS. 8A-9R. When shunt device 400 is released from a delivery catheter to be deployed in a puncture in a tissue wall, distal arms 430 are released first on a first side of the tissue wall. For example, when shunt device 400 is delivered into the tissue wall between a left atrium and a coronary sinus of a heart, distal arms 430 can contact the left atrial side of the tissue wall. A physician who is operating the delivery catheter can then pull back on the delivery catheter to confirm whether distal arms 430 are in the correct position and have contacted (i.e., captured) tissue. When the delivery catheter is pulled back, distal arm 430B including lengthened portion 460 (the lengthened arm 414) will deflect away from the tissue wall. That is, the lengthened arm 414 including lengthened portion 460 is deflectable along a deflection arc away from horizontal reference plane HP. A central angle of the deflection arc can be between zero (0°) and ninety degrees (90°) as measured from horizontal reference plane HP. For example, the design of lengthened arm 414 can be calibrated so that its deflection does not cause it to fully straighten out (the central angle being ninety degrees), which could cause shunt device 400 to become displaced. Deflection of the lengthened arm 414 can be detected with fluoroscopy or other real-time imaging techniques. For example, a physician can track deflection of the lengthened arm 414 by visualizing a change in the location of radiopaque markers 418 with fluoroscopic imaging.

The lengthened arm 414 including lengthened portion 460 improves responsiveness during pull back of the delivery catheter used for implantation of shunt device 400. Lengthened portion 460 permits increased deflection of the lengthened arm 414, which can be more easily detected during the implantation procedure. The lengthened arm 414 is also longer due to the inclusion of lengthened portion 460, so the lengthened arm 414 can tolerate a greater amount of displacement before the arm will slip into an incorrect position and potentially cause shunt device 400 to become mis-seated or embolized. Lengthened portion 460 allows the lengthened arm 414 to be sensitive enough to deflect but also robust enough to withstand fatigue and strain from, e.g., implantation of shunt device 400 or forces within the heart. Accordingly, shunt device 400 including lengthened portion 460 has improved tissue capture characteristics compared to traditional shunt devices.

### Shunt Devices 500 and 500′ (FIGS. 11A-12B)

FIG. 11A is a perspective view of shunt device 500 including tabs 565. FIG. 11B is a flattened view of shunt device 500 including tabs 565. FIGS. 11A-11B will be described together. As illustrated in FIGS. 11A-11B, shunt device 500 includes body 502, which is formed of struts 504 and openings 506. Body 502 includes central flow tube 510, flow path 512, and arms 514. Shunt device 500 also includes tissue capture features 516 and radiopaque markers 518. Central flow tube 510 has side portions 520 (including side portion 520A and side portion 520B), end portions 522 (including end portion 522A and end portion 522B), first axial end 524, and second axial end 526. Arms 514 include distal arms 530 (including distal arm 530A and distal arm 530B) and proximal arms 532 (including proximal arm 532A and proximal arm 532B). Distal arms 530 have terminal ends 534 (including terminal end 534A and terminal end 534B). Proximal arms 532 have terminal ends 536 (including terminal end 536A and terminal end 536B). As is further illustrated in FIGS. 11A-11B, tissue capture features 516 include tabs 565. FIG. 11B shows a flattened view of shunt device 500 as though shunt device 500 has been separated or cut along a cut line (not shown) drawn longitudinally through distal arm 530B, through end portion 522B, and longitudinally through proximal arm 532B.

Shunt device 500 has a generally similar structure and design to shunt devices 100 and 100′ described above in reference to FIGS. 3A-5, and shunt device 500 includes tabs 565. Tabs 565 are described here with respect to shunt device 500 but can also be included on any of the examples of a shunt device descried herewith, including shunt devices 100, 100′, 400, 500′, 600, 600′, 600″, 700, 700′, 800, 800′, 800″, 900, and 900′.

Tabs 565 are another form of tissue capture features 516. Tabs 565 are elongated projections that extend radially outward from central flow tube 510. For example, as shown in FIGS. 11A-11B, tabs 565 can be attached to first axial end 524. In other examples, tabs 565 can be attached to second axial end 526. In yet other examples, tabs 565 can be attached to each of first axial end 524 and second axial end 526. More specifically, tabs 565 can be attached to side portions 520. For example, as shown in FIGS. 11A-11B, a first one of tabs 565 is attached to side portion 520A and a second one of tabs 565 is attached to side portion 520B, such that shunt device 500 includes two tabs 565. In other examples, shunt device 500 can include any suitable number of tabs 565, including more or fewer tabs 565 attached to ones of side portions 520. In some examples, shunt device can include a single tab 565 on either side portion 520.

Tabs 565 are formed of struts 504 like body 502. Tabs 565 can be generally rectangular or oblong in shape. For example, tabs 565 can include struts 504 framing a rectangular opening 506. In other examples, tabs 565 can be any other suitable shapes. In yet other examples, tabs 565 can be formed of a single, thicker strut 504 that does not have an opening therein. Tabs 565 can also be slightly curled or arched away from central flow tube 510, like arms 414, when shunt device 500 is in an expanded configuration. Compared to arms 514, however, tabs 565 may be relatively shorter and narrower to fit along side portions 520 between distal arms 530 or proximal arms 532. In general, the physical dimensions of tabs 565 can be selected to avoid interaction between tabs 565 and nearby anatomical features when shunt device 500 is implanted in a puncture in a tissue wall. For example, tabs 565 can be sized to avoid interaction with a mitral valve in a left atrium of a heart. The physical dimensions of tabs 565 can also be selected based on fatigue and strain considerations.

Tabs 565 can be formed of any suitable material, including a shape-memory material such as nitinol (a nickel titanium alloy). Tabs 565 can be formed of a same material as body 502. For example, tabs 565 can be part of a single laser cut pattern for shunt device 500 such that shunt device 500 including tabs 565 is a monolithic structure. In other examples, tabs 565 can be welded or otherwise attached to central flow tube 510. Alternatively, tabs 565 can be formed of a different material from body 502. In such examples, tabs 565 can be welded or otherwise attached to central flow tube 510.

Radiopaque markers 518 can be included on shunt device 500. Radiopaque markers 518 are structures that are dense and resist the passage of X-rays to permit visualization with radiographic imaging. For example, radiopaque markers 518 can be attached or stamped at an end of each of tabs 565 that is distal to central flow tube 510. In some examples, radiopaque markers 518 can also be attached at multiple ones of terminal ends 534, 536 and to other tissue capture features 516. For example, FIGS. 11A-11B show radiopaque markers 518 at terminal ends 534B and 536B and associated with each of tabs 565. In other examples, radiopaque markers 518 can be attached to any suitable portion or portions of shunt device 500, such as other locations on arms 514 or other locations on tissue capture features 516. Moreover, shunt device 500 can include any suitable number of radiopaque markers 518. In yet other examples, shunt device 500 may not include any radiopaque markers 518. Radiopaque markers 518 can be shaped and sized to fit on the portions of shunt device 500 to which they are attached. In other examples, radiopaque markers 518 can be thickened regions of struts 504 rather than separate pieces that are attached. For example, shunt device 500 can be formed of nitinol (a nickel titanium alloy) and radiopaque markers 518 can be relatively thickened regions of nitinol.

FIG. 12A is a top view of shunt device 500′ including tabs 565′. FIG. 12B is a flattened view of shunt device 500′ including tabs 565′. FIGS. 12A-12B will be described together. As illustrated in FIGS. 12A-12B, shunt device 500′ includes body 502′, which is formed of struts 504′ and openings 506′. Body 502′ includes central flow tube 510′, flow path 512′, and arms 514′. Shunt device 500′ also includes tissue capture features 516′ and radiopaque markers 518′. Central flow tube 510′ has side portions 520′ (including side portion 520A′ and side portion 520B′), end portions 522′ (including end portion 522A′ and end portion 522B′), first axial end 524′, and second axial end 526′. Arms 514′ include distal arms 530′ (including distal arm 530A′ and distal arm 530B′) and proximal arms 532′ (including proximal arm 532A′ and proximal arm 532B′). Distal arms 530′ have terminal ends 534′ (including terminal end 534A′ and terminal end 534B′). Proximal arms 532′ have terminal ends 536′ (including terminal end 536A′ and terminal end 536B′). As is further illustrated in FIGS. 12A-12B, tissue capture features 516′ include tabs 565′. FIG. 12B shows a flattened view of shunt device 500′ as though shunt device 500′ has been separated or cut along a cut line (not shown) drawn longitudinally through distal arm 530B′, through end portion 522B′, and longitudinally through proximal arm 532B′.

Shunt device 500′ has a generally similar structure and design to shunt device 500 described above, except shunt device 500′ includes tabs 565′ instead of tabs 565. Compared to tabs 565 shown in FIGS. 11A-11B, tabs 565′ are relatively longer and narrower. Additionally, tabs 565′ do not include radiopaque markers (radiopaque markers 518′ are shown in FIGS. 12A-12B on terminal ends 534A′, 534B′, and 536B′ and associated with other tissue capture features 516′).

Shunt devices 500 and 500′ can be delivered into a human body using, for example, delivery catheter 200 (as shown in FIGS. 6-7B) and according to, for example, method 300 described above in reference to FIGS. 8A-9R. The operation of shunt devices 500 and 500′ will be described with reference to FIGS. 11A-12B together. When shunt devices 500 and 500′ are released from a delivery catheter to be deployed in a puncture in a tissue wall, tabs 565 and 565′ will flare outwards from respective central flow tubes 510 and 510′ to lay relatively flat against a side of the tissue wall. Tabs 565 and 565′ can be held in a straightened configuration when shunt devices 500 and 500′ are still in the delivery catheter. Tabs 565 and 565′ attached to respective first axial ends 524 and 524′ can contact a first side of the tissue wall, whereas tabs 565 and 565′ attached to respective second axial ends 526 and 526′ can contact a second side of the tissue wall. For example, when shunt devices 500 and 500′ are inserted into a puncture in the tissue wall between a left atrium and a coronary sinus of a heart, tabs 565 and 565′ attached to respective first axial ends 524 and 524′ can contact the left atrial side of the tissue wall, and tabs 565 and 565′ attached to respective second axial ends 526 and 526′ can contact the coronary sinus side of the tissue wall. Pressures in the left atrium can help push tabs 565 and 565′ against the left atrial side of the tissue wall to keep shunt devices 500 and 500′ in place. Tabs 565 and 565′ are located on at least one side of shunt devices 500 and 500′ (e.g., respective first axial ends 524 and 524′ and/or second axial ends 526 and 526′) to prevent shunt devices 500 and 500′ from slipping out of the puncture in the tissue wall and becoming embolized within a chamber or vessel of the heart.

In general, tabs 565 and 565′ increase the stability of shunt devices 500 and 500′ compared to devices without tabs because there are more parts of shunt devices 500 and 500′ gripping the tissue wall. More specifically, tabs 565 and 565′ are attached to respective side portions 520 and 520′ to provide lateral support for shunt devices 500 and 500′. That is, arms 514 and 514′ are all aligned generally along one axis drawn horizontally through respective central flow tubes 510 and 510′, but tabs 565 and 565′ can extend radially from the circumference of respective central flow tubes 510 and 510′ along different axes. In this way, tabs 565 and 565′ function as supplementary stabilizing arms for shunt devices 500 and 500′ to provide stability in directions different from the support provided by arms 514 and 514′. Accordingly, shunt devices 500 and 500′ including tabs 565 and 565′ have improved tissue capture characteristics compared to traditional shunt devices.

Additionally, including radiopaque markers 518 and 518′ on tabs 565 and 565′ provides additional visual indication of shunt positioning during and/or after implantation of shunt devices 500 and 500′. Including radiopaque markers 518 and 518′ on tabs 565 and 565′ can provide a central visual reference for positioning of shunt devices 500 and 500′, in addition to the reference points from radiopaque markers 518 and 518′ that are attached distally on arms 514 and 514′.

### Shunt Devices 600, 600′, and 600″ (FIGS. 13A-16B)

FIG. 13A is a top view of shunt device 600 including deflectable projection 670. FIG. 13B is a flattened view of shunt device 600 including deflectable projection 670. FIGS. 13A-13B will be described together. As illustrated in FIGS. 13A-13B, shunt device 600 includes body 602, which is formed of struts 604 and openings 606. Body 602 includes central flow tube 610, flow path 612, and arms 614. Shunt device 600 also includes tissue capture features 616 and radiopaque markers 618. Central flow tube 610 has side portions 620 (including side portion 620A and side portion 620B), end portions 622 (including end portion 622A and end portion 622B), first axial end 624, and second axial end 626. Arms 614 include distal arms 630 (including distal arm 630A and distal arm 630B) and proximal arms 632 (including proximal arm 632A and proximal arm 632B). Distal arms 630 have terminal ends 634 (including terminal end 634A and terminal end 634B). Proximal arms 632 have terminal ends 636 (including terminal end 636A and terminal end 636B). As is further illustrated in FIGS. 13A-13B, tissue capture features 616 include deflectable projection 670, which includes attachment end 672 and terminal end 674. FIG. 13B shows a flattened view of shunt device 600 as though shunt device 600 has been separated or cut along a cut line (not shown) drawn longitudinally through distal arm 630B, through end portion 622B, and longitudinally through proximal arm 632B.

Shunt device 600 has a generally similar structure and design to shunt devices 100 and 100′ described above in reference to FIGS. 3A-5, however shunt device 600 also includes deflectable projection 670. Deflectable projection 670 is described here with respect to shunt device 600 but can also be included on any of the examples of a shunt device descried herewith, including shunt devices 100, 100′, 400, 500, 500′, 600′, 600″, 700, 700′, 800, 800′, 800″, 900, and 900′.

Deflectable projection 670 is another form of tissue capture features 616. Deflectable projection 670 is an elongated projection connected to at least one of arms 614. In one example, deflectable projection 670 can be connected to one or more of distal arms 630, such as distal arm 630B (as shown in FIGS. 13A-13B and 14). In other examples, deflectable projection 670 can be connected to one or more of proximal arms 632 (e.g., as shown in FIG. 15). In yet other examples, shunt device 600 can include multiple deflectable projections 670 connected to multiple ones of arms 614 (e.g., as shown in FIGS. 16A-16B). Generally, deflectable projections 670 can be attached to any, some, or all of arms 614. In some examples, deflectable projections 670 can be attached to ones of arms 614 for which a physician includes a step to confirm tissue contact has been made during an implantation procedure.

Deflectable projection 670 is connected to or originates from one of arms 614 at attachment end 672. Deflectable projection 670 extends from attachment end 672 to terminal end 674. In one example, attachment end 672 can be connected to one of arms 614 at a location distal to or away from central flow tube 610, and deflectable projection 670 can extend radially inward toward central flow tube 610 such that terminal end 674 is proximal to central flow tube 610. Alternatively, attachment end 672 can be connected to one of arms 614 at a location proximal to central flow tube 610, and deflectable projection 670 can extend radially outward toward a respective terminal end 634, 646 of the one of arms 614 such that terminal end 674 of deflectable projection 670 is proximal to the respective terminal end 634, 636. In some examples, deflectable projection 670 extends within a perimeter (or envelope) of the one of arms 614 to which it is attached. In other words, as is most easily seen in FIG. 13B, struts 604 of the one of arms 614 to which deflectable projection 670 is attached can surround deflectable projection 670.

Deflectable projection 670 is compliant and easily deflected upon contact with tissue. Deflectable projection 670 is more compliant or more sensitive to deflection than the one of arms 614 to which it is attached. That is, deflectable projection 670 will deflect before arms 614 as increasing pressure is applied to deflectable projection 670 and arms 614. To be more compliant, deflectable projection 670 can be formed of longer and thinner struts 604 compared to arms 614. Struts 604 of deflectable projection 670 can also form winding patterns with several hairpin curved portions to increase the total length of the struts. Deflectable projections 670 with longer, thinner struts 604 will be softer and more compliant than deflectable projections 670 with thicker, shorter struts 604. These variations in the design of deflectable projection 670 can be used to obtain a desired sensitivity of deflectable projection 670, which may vary depending on characteristics of the particular tissue in which shunt device 600 will be implanted, other anatomical or physical characteristics, characteristics of shunt device 600, etc.

In general, the physical dimensions of deflectable projection 670 can be selected to avoid interaction between deflectable projection 670 and nearby anatomical features when shunt device 600 is implanted in a puncture in a tissue wall. For example, deflectable projection 670 can be sized to avoid interaction with a mitral valve in a left atrium of a heart. The physical dimensions of deflectable projection 670 can also be selected based on a desired amount of deflection of deflectable projection 670 balanced against fatigue and strain considerations. The desired amount of deflection can be calibrated for optimal visualization during and/or after implantation of shunt device 600.

Deflectable projection 670 can be formed of any suitable material, including a shape-memory material such as nitinol (a nickel titanium alloy). Deflectable projection 670 can be formed of a same material as body 602. For example, deflectable projection 670 can be part of a single laser cut pattern for shunt device 600 such that shunt device 600 including deflectable projection 670 is a monolithic structure. In other examples, deflectable projection 670 can be welded or otherwise attached to one of arms 614. Alternatively, deflectable projection 670 can be formed of a different material from body 602. In such examples, deflectable projection 670 can be welded or otherwise attached to one of arms 614.

Radiopaque markers 618 can be included on shunt device 600. Radiopaque markers 618 are structures that are dense and resist the passage of X-rays to permit visualization with radiographic imaging. For example, radiopaque markers 618 can be attached or stamped at terminal end 674 of deflectable projection 670. In some examples, radiopaque markers 618 can also be attached at multiple ones of terminal ends 634, 636 and to other tissue capture features 616. For example, FIGS. 13A-13B show radiopaque markers 618 at terminal ends 534A, 534B, and 536B and associated with deflectable projection 670. In other examples, radiopaque markers 618 can be attached to any suitable portion or portions of shunt device 600, such as other locations on arms 614 or other locations on tissue capture features 616. Moreover, shunt device 600 can include any suitable number of radiopaque markers 618. In yet other examples, shunt device 600 may not include any radiopaque markers 618. Radiopaque markers 618 can be shaped and sized to fit on the portions of shunt device 600 to which they are attached. In other examples, radiopaque markers 618 can be thickened regions of struts 604 rather than separate pieces that are attached. For example, shunt device 600 can be formed of nitinol (a nickel titanium alloy) and radiopaque markers 618 can be relatively thickened regions of nitinol.

FIG. 14 is a flattened view of shunt device 600′ including deflectable projection 670′. As illustrated in FIG. 14, shunt device 600′ includes body 602′, which is formed of struts 604′ and openings 606′. Body 602′ includes central flow tube 610′, and arms 614′. Shunt device 600′ also includes tissue capture features 616′ and radiopaque markers 618′. Central flow tube 610′ has side portions 620′ (including side portion 620A′ and side portion 620B′), end portions 622′ (including end portion 622A′ and end portion 622B′), first axial end 624′, and second axial end 626′. Arms 614′ include distal arms 630′ (including distal arm 630A′ and distal arm 630B′) and proximal arms 632′ (including proximal arm 632A′ and proximal arm 632B′). Distal arms 630′ have terminal ends 634′ (including terminal end 634A′ and terminal end 634B′). Proximal arms 632′ have terminal ends 636′ (including terminal end 636A′ and terminal end 636B′). As is further illustrated in FIG. 14, tissue capture features 616′ include deflectable projection 670′. FIG. 14 shows a flattened view of shunt device 600′ as though shunt device 600′ has been separated or cut along a cut line (not shown) drawn longitudinally through distal arm 630B′, through end portion 622B′, and longitudinally through proximal arm 632B′.

Shunt device 600′ has a generally similar structure and design to shunt device 600 described above, except shunt device 600′ includes deflectable projection 670′ instead of deflectable projection 670. Compared to deflectable projection 670 as shown in FIGS. 13A-13B, deflectable projection 670′ is formed of irregularly shaped and curved struts 604′, which form curved hairpin turns rather than rectangular ones. The irregular curved shape of deflectable projection 670′ can affect the sensitivity of deflectable projection 670′. The curved portions may also be desirable to prevent any sharp edges damaging adjacent tissue walls when shunt device 600′ is implanted or to prevent shunt device 600′ including deflectable projection 670′ from becoming snagged or caught in a delivery catheter. FIG. 14 also shows shunt device 600′ including deflectable projection 670′ in combination with other tissue capture features 616′.

FIG. 15 is a flattened view of shunt device 600″ including deflectable projection 670″. As illustrated in FIG. 15, shunt device 600″ includes body 602″, which is formed of struts 604″ and openings 606″. Body 602″ includes central flow tube 610″, and arms 614″. Shunt device 600″ also includes tissue capture features 616″ and radiopaque markers 618″. Central flow tube 610″ has side portions 620″ (including side portion 620A″ and side portion 620B″), end portions 622″ (including end portion 622A″ and end portion 622B″), first axial end 624″, and second axial end 626″. Arms 614″ include distal arms 630″ (including distal arm 630A″ and distal arm 630B″) and proximal arms 632″ (including proximal arm 632A″ and proximal arm 632B″). Distal arms 630″ have terminal ends 634″ (including terminal end 634A″ and terminal end 634B″). Proximal arms 632″ have terminal ends 636″ (including terminal end 636A″ and terminal end 636B″). As is further illustrated in FIG. 15, tissue capture features 616″ include deflectable projection 670″. FIG. 15 shows a flattened view of shunt device 600″ as though shunt device 600″ has been separated or cut along a cut line (not shown) drawn longitudinally through distal arm 630B″, through end portion 622B″, and longitudinally through proximal arm 632B″.

Shunt device 600″ has a generally similar structure and design to shunt device 600 described above, except shunt device 600″ includes deflectable projection 670″ instead of deflectable projection 670. Compared to deflectable projection 670 as shown in FIGS. 13A-13B, deflectable projection 670″ is formed of a single, straight strut 604″. Strut 604″ of deflectable projection 670″ is also thicker than those of deflectable projection 670. Accordingly, deflectable projection 670″ can be less sensitive and require greater pressure to deflect compared to deflectable projection 670. Additionally, deflectable projection 670″ is shown attached to proximal arm 632A″, rather than one of distal arms 630″, so deflectable projection 670″ can contact an opposite side of a tissue wall with respect to an orientation of shunt device 600″ when it is implanted in a puncture in the tissue wall.

FIG. 16A is a schematic view of shunt device 600 including deflectable projection 670 in a relaxed state. FIG. 16B is a schematic view of shunt device 600 including deflectable projection 670 in a deflected state. FIGS. 16A-16B show sensor device 600, arms 614, radiopaque markers 618, and deflectable projections 670. FIG. 16A further shows first gap G1, and FIG. 16B further shows second gap G2 and tissue wall TW.

Shunt devices 600, 600′, and 600″ can be delivered into a human body using, for example, delivery catheter 200 (as shown in FIGS. 6-7B) and according to, for example, method 300 described above in reference to FIGS. 8A-9R. The operation of shunt devices 600, 600′, and 600″ will be described with reference to FIGS. 13A-16B together. For ease of discussion, functionality will be described with respect to shunt device 600 and its components; however, it should be understood that the functionality described herein is applicable to each of shunt devices 600, 600′, and 600″, except where a difference is indicated.

FIG. 16A shows shunt device 600 in a relaxed state, e.g., when shunt device 600 is not implanted in a puncture in a tissue wall or when shunt device 600 is at least partially displaced from the puncture. As shown in FIG. 16A, deflectable projections 670 are biased away from the ones of arms 614 to which they are attached such that first gaps G1 are formed therebetween when viewed from the side. Thus, gap G1 can be an initial or baseline gap between deflectable projections 670 and corresponding ones of arms 614. The height of gap G1 can be configured depending on a desired amount of deflection, e.g., for optimal visualization, and balanced against fatigue and strain considerations.

As shown in FIG. 16B, deflectable projections 670 are configured to deflect toward the respective ones of arms 614 when shunt device 600 is implanted in a puncture in tissue wall TW and arms 614 have contacted (i.e., captured) tissue. Accordingly, FIG. 16B shows shunt device 600 with deflectable projections 670 in a deflected state. The surface of tissue wall TW that is in contact with deflectable projections 670 presses deflectable projections 670 toward the respective arms 614. When deflectable projections 670 deflect toward the respective arms 614, the gaps between them are reduced or closed, depending on the positioning and sensitivity of deflectable projections 670. As such, gaps G2 are formed therebetween instead of gap G1, and gaps G2 are smaller than gap G1. In some examples, gaps G2 may be closed completely such that deflectable projections 670 are pressed flat against the respective ones of arms 614.

Deflection of deflectable projection 670 with respect to one of arms 614 can provide visual evidence that arms 614 are properly placed and/or that shunt device 600 is not mis-seated or embolized. Deflection of deflectable projection 670 can be detected with fluoroscopy or other real-time imaging techniques. For example, a physician can track deflection of deflectable projection 670 by visualizing a change in the location of radiopaque markers 618 with fluoroscopic imaging. Specifically, when deflectable projection 670 and the respective arm 614 each include radiopaque markers 618, there will be relative movement between the markers when tissue has been successfully captured and deflectable projection 670 deflects. Depending on the initial configuration, radiopaque markers 618 attached to deflectable projection 670 and the respective arm 614 can be in a same plane when shunt device 600 is in the relaxed state and move out of plane when deflectable projection 670 deflects. Alternatively, radiopaque markers 618 attached to deflectable projection 670 and the respective arm 614 may not be aligned in the same plane when shunt device 600 is in the relaxed state and move into alignment when deflectable projection 670 deflects. The desired relative position in the deflected state of radiopaque markers 618 included on deflectable projection 670 and/or the respective arm 614 can also be configured based on the shape set of the arms.

Deflectable projections 670, 670′, and 670″ can provide improved visualization of tissue capture for shunt devices 600, 600′, and 600″ in situations where the location of shunt devices 600, 600′, and 600″, or a portion of shunt devices 600, 600′, and 600″, such as ones of arms 614, 614′, and 614″, is difficult to determine from an isolated visual marker. This is because deflectable projections 670, 670′, and 670″ provide for visualization of relative movement compared to a non-deflecting arm. Accordingly, shunt devices 600, 600′, and 600″ including deflectable projections 670, 670′, and 670″ have improved tissue capture characteristics compared to traditional shunt devices.

### Shunt Devices 700 and 700′ (FIGS. 17A-19B)

FIG. 17A is a side view of shunt device 700 including secondary arm 775. FIG. 17B is a top view of the first example of shunt device 700 including secondary arm 775. FIG. 17C is a flattened view of shunt device 700 including secondary arm 775. FIGS. 17A-17C will be described together. As illustrated in FIGS. 17A-17C, shunt device 700 includes body 702, which is formed of struts 704 and openings 706. Body 702 includes central flow tube 710, flow path 712, and arms 714. Shunt device 700 also includes tissue capture features 716 and radiopaque markers 718. Central flow tube 710 has side portions 720 (including side portion 720A and side portion 720B), end portions 722 (including end portion 722A and end portion 722B), first axial end 724, and second axial end 726. Arms 714 include distal arms 730 (including distal arm 730A and distal arm 730B) and proximal arms 732 (including proximal arm 732A and proximal arm 732B). Distal arms 730 have terminal ends 734 (including terminal end 734A and terminal end 734B). Proximal arms 732 have terminal ends 736 (including terminal end 736A and terminal end 736B). As is further illustrated in FIGS. 17A-17C, tissue capture features 716 include secondary arm 775, which includes attachment end 776 and terminal end 778. FIG. 17A further shows horizontal reference plane HP, perpendicular reference axis RA, central axis CA, tilt angle θ, first angle α, and second angle β. FIG. 17C shows a flattened view of shunt device 700 as though shunt device 700 has been separated or cut along a cut line (not shown) drawn longitudinally through distal arm 730B, through end portion 722B, and longitudinally through proximal arm 732B.

Shunt device 700 has a generally similar structure and design to shunt devices 100 and 100′ described above in reference to FIGS. 3A-5, however shunt device 700 also includes secondary arm 775. Secondary arm 775 is described here with respect to shunt device 700 but can also be included on any of the examples of a shunt device descried herewith, including shunt devices 100, 100′, 400, 500, 500′, 600, 600′, 600″, 700′, 800, 800′, 800″, 900, and 900′.

Secondary arm 775 is another form of tissue capture features 716. Secondary arm 775 is an additional arm or arm portion associated with at least one of arms 714. In one example, secondary arm 775 can be associated with one or more of distal arms 730, such as distal arm 730B (as shown in FIGS. 17A-17C). In other examples, secondary arm 775 can be associated with one or more of proximal arms 732. In yet other examples, shunt device 700 can include multiple secondary arms 775 associated with multiple ones of arms 714. Generally, secondary arms 775 can be associated with any, some, or all of arms 714. In some examples, secondary arms 775 can be associated with ones of arms 714 for which a physician includes a step to confirm tissue contact has been made during an implantation procedure.

Secondary arm 775 is connected to or originates from central flow tube 710 at attachment end 776. More specifically, secondary arm 775 is connected to one of the opposed pair of end portions 722 of central flow tube 710. Secondary arm 775 extends from attachment end 776 to terminal end 778. Secondary arm 775 extends radially outward from central flow tube 710 with one of arms 714. That is, secondary arm 775 generally extends along the same path (axis) as the one of arms 714 with which it is associated. Secondary arm 775 is similar to deflectable projection 670 (as shown, e.g., in FIGS. 13A-13B), except secondary arm 775 originates from central flow tube 710 adjacent one of arms 714. Terminal end 778 of secondary arm 775 can be proximal to a respective terminal end 734, 736 of the one of arms 714. In some examples, secondary arm 775 extends within a perimeter (or envelope) of the one of arms 714 with which it is associated. In other words, as is most easily seen in FIG. 17C, struts 704 of the one of arms 714 with which secondary arm 775 is associated can surround secondary arm 670.

Between secondary arm 775 and the one of arms 714 with which it is associated, either secondary arm 775 or the one of arms 714 will be compliant and easily deflected upon contact with tissue. In one example, secondary arm 775 is more compliant than the one of arms 714 with which it is associated. That is, secondary arm 775 will deflect before the one of arms 714 as increasing pressure is applied to secondary arm 775 and the one of arms 714. Alternatively, secondary arm 775 is less compliant than the one of arms 714 with which it is associated. That is, the one of arms 714 will deflect before secondary arm 775 as increasing pressure is applied to secondary arm 775 and the one of arms 714. The more compliant one of secondary arm 775 and the one of arms 714 with which it is associated can be formed of relatively longer, thinner struts 704 and/or fewer struts 704. On the other hand, the less compliant one of secondary arm 775 and the one of arms 714 with which it is associated can be formed of relatively thicker, stiffer struts 704 and/or more struts 704. Variations in the pattern (e.g., length, thickness, number) of struts 704 of secondary arm 775 and the one of arms 714 with which it is associated can be used to select a desired sensitivity or level of relative deflection between the two arms, which may vary depending on characteristics of the particular tissue in which shunt device 700 will be implanted, other anatomical or physical characteristics, characteristics of shunt device 700, etc.

In general, the physical dimensions of secondary arm 775 can be selected to avoid interaction between secondary arm 775 and nearby anatomical features when shunt device 700 is implanted in a puncture in a tissue wall. For example, secondary arm 775 can be sized to avoid interaction with a mitral valve in a left atrium of a heart. The physical dimensions of secondary arm 775 can also be selected based on a desired amount of deflection of secondary arm 775 balanced against fatigue and strain considerations. The desired amount of deflection can be calibrated for optimal visualization during and/or after implantation of shunt device 700.

Secondary arm 775 can be formed of any suitable material, including a shape-memory material such as nitinol (a nickel titanium alloy). Secondary arm 775 can be formed of a same material as body 702. For example, secondary arm 775 can be part of a single laser cut pattern for shunt device 700 such that shunt device 700 including secondary arm 775 is a monolithic structure. In other examples, secondary arm 775 can be welded or otherwise attached to central flow tube 710. Alternatively, secondary arm 775 can be formed of a different material from body 702. In such examples, secondary arm 775 can be welded or otherwise attached to central flow tube 710.

Radiopaque markers 718 can be included on shunt device 700. Radiopaque markers 718 are structures that are dense and resist the passage of X-rays to permit visualization with radiographic imaging. For example, radiopaque markers 718 can be attached or stamped at terminal end 778 of secondary arm 775. In some examples, radiopaque markers 718 can also be attached at multiple ones of terminal ends 734, 736 and to other tissue capture features 716. For example, FIGS. 17A-17C show radiopaque markers 718 at terminal ends 734A, 734B, and 736B and associated with secondary arm 775. In other examples, radiopaque markers 718 can be attached to any suitable portion or portions of shunt device 700, such as other locations on arms 714 or other locations on tissue capture features 716. Moreover, shunt device 700 can include any suitable number of radiopaque markers 718. In yet other examples, shunt device 700 may not include any radiopaque markers 718. Radiopaque markers 718 can be shaped and sized to fit on the portions of shunt device 700 to which they are attached. In other examples, radiopaque markers 718 can be thickened regions of struts 704 rather than separate pieces that are attached. For example, shunt device 700 can be formed of nitinol (a nickel titanium alloy) and radiopaque markers 718 can be relatively thickened regions of nitinol.

FIG. 18 is a flattened view of shunt device 700′ including secondary arm 775′. As illustrated in FIG. 18, shunt device 700′ includes body 702′, which is formed of struts 704′ and openings 706′. Body 702′ includes central flow tube 710′, and arms 714′. Shunt device 700′ also includes tissue capture features 716′ and radiopaque markers 718′. Central flow tube 710′ has side portions 720′ (including side portion 720A′ and side portion 720B′), end portions 722′ (including end portion 722A′ and end portion 722B′), first axial end 724′, and second axial end 726′. Arms 714′ include distal arms 730′ (including distal arm 730A′ and distal arm 730B′) and proximal arms 732′ (including proximal arm 732A′ and proximal arm 732B′). Distal arms 730′ have terminal ends 734′ (including terminal end 734A′ and terminal end 734B′). Proximal arms 732′ have terminal ends 736′ (including terminal end 736A′ and terminal end 736B′). As is further illustrated in FIG. 18, tissue capture features 716′ include secondary arm 775′. FIG. 18 shows a flattened view of shunt device 700′ as though shunt device 700′ has been separated or cut along a cut line (not shown) drawn longitudinally through distal arm 730B′, through end portion 722B′, and longitudinally through proximal arm 732B′.

Shunt device 700′ has a generally similar structure and design to shunt device 700 described above, except shunt device 700′ includes secondary arm 775′ instead of secondary arm 775. Compared to secondary arm 775 as shown in FIGS. 17A-17B, secondary arm 775′ includes a sensor attachment portion (e.g., sensor attachment portion 952 as described below with respect to FIGS. 24A-25C) for attaching to a sensor. As shown in the example of FIG. 18, secondary arm 775′ is associated with distal arm 730A′. Secondary arm 775′ can be more rigid or less compliant compared to distal arm 730A′ so that secondary arm 775′ can support a sensor attached thereto. Accordingly, distal arm 730A′ can be relatively more compliant. For example, as illustrated in FIG. 18, distal arm 730A′ can be formed of a single, relatively thinner strut 704′.

FIG. 19A is a schematic view of shunt device 700 including secondary arm 775 in a relaxed state. FIG. 19B is a schematic view of shunt device 700 including secondary arm 775 in a deflected state. FIGS. 19A-19B show sensor device 700, arm 714, radiopaque markers 718, and secondary arm 775. FIG. 19A further shows initial gap GI, and FIG. 19B further shows tissue wall TW.

Shunt devices 700 and 700′ can be delivered into a human body using, for example, delivery catheter 200 (as shown in FIGS. 6-7B) and according to, for example, method 300 described above in reference to FIGS. 8A-9R. The operation of shunt devices 700 and 700′ will be described with reference to FIGS. 17A-19B together. For ease of discussion, functionality will be described with respect to shunt device 700 and its components; however, it should be understood that the functionality described herein is applicable to each of shunt devices 700 and 700′, except where a difference is indicated.

FIG. 19A shows shunt device 700 in a relaxed state, e.g., when shunt device 700 is not implanted in a puncture in a tissue wall or when shunt device 700 is at least partially displaced from the puncture. As shown in FIG. 19A, secondary arm 775 is biased away from the one of arms 714 with which it is associated such that initial gap GI is formed therebetween when viewed from the side. Thus, initial gap GI can be an initial or baseline gap between secondary arm 775 and the corresponding one of arms 714. The height of gap G1 can be configured depending on a desired amount of deflection, e.g., for optimal visualization, and balanced against fatigue and strain considerations.

FIG. 19B shows shunt device 600 with secondary arm 775 and a corresponding one of arms 714 in a deflected state. More specifically, the progression from FIG. 19A to FIG. 19B shows an example where secondary arm 775 is less compliant than the one of arms 714 with which it is associated. Accordingly, as shown in FIG. 19B, the corresponding one of arms 714 with which secondary arm 775 is associated is configured to deflect toward secondary arm 775 when shunt device 700 is implanted in a puncture in tissue wall TW and arms 714 have contacted (i.e., captured) tissue. The surface of tissue wall TW that is in contact with the respective arm 714 presses arm 714 toward secondary arm 775. When arm 714 deflects toward secondary arm 775, the gap between them is closed. As such, there is no gap between arm 714 and secondary arm 775 in the deflected state shown in FIG. 19B.

In an alternative example where secondary arm 775 is instead more compliant than the one of arms 714 with which it is associated, secondary arm 775 would be configured to deflect toward the corresponding one of arms 714 when shunt device 700 is implanted in a puncture in tissue wall TW and arms 714 have contacted (i.e., captured) tissue. A version of FIG. 19A showing a relaxed state of such an example of shunt device 700 would have the respective orientation of secondary arm 775 and arm 714 switched so that secondary arm 775 would be on the bottom and arm 714 would be on the top, as viewed like in FIG. 19A. The surface of tissue wall TW that contacts the more compliant secondary arm 775 would press secondary arm 775 toward the respective one of arms 714. When the more compliant secondary arm 775 deflects toward arm 714, the gap between them would close, leaving no gap between arm 714 and secondary arm 775 in the deflected state.

In general, the less compliant (or more rigid) one of secondary arm 775 and arm 714 is configured to hold shunt device 700 in place against tissue wall TW. On the other hand, the more compliant (or softer) one of secondary arm 775 and arm 714 is configured to provide relative motion for visualization purposes.

Deflection of secondary arm 775 with respect to one of arms 714 when secondary arm 775 is more compliant, or deflection of a corresponding one of arms 714 with respect to secondary arm 775 when arm 714 is more compliant, can provide visual evidence that arms 714 are properly placed and/or that shunt device 700 is not mis-seated or embolized. Deflection of secondary arm 775 or the corresponding one of arms 714 can be detected with fluoroscopy or other real-time imaging techniques. For example, a physician can track deflection of secondary arm 775 or the corresponding one of arms 714 by visualizing a change in the location of radiopaque markers 718 with fluoroscopic imaging. Specifically, when secondary arm 775 and the corresponding one of arms 714 each include radiopaque markers 718, there will be relative movement between the markers when tissue has been successfully captured and either secondary arm 775 or arm 714 deflects. Radiopaque markers 718 attached to secondary arm 775 and the corresponding arm 714 are not aligned when shunt device 600 is in the relaxed state but move into alignment when secondary arm 775 or arm 714 deflects. In examples where secondary arm 775 is within the perimeter of the corresponding arm 714, radiopaque markers attached to respective terminal ends 778 and 734, 736 can be aligned in the same plane when secondary arm 775 or arm 714 deflects. In other examples, radiopaque markers attached to respective terminal ends 778 and 734, 736 can be configured to overlap when secondary arm 775 or arm 714 deflects. The desired relative position in the deflected state of radiopaque markers 718 included on secondary arm 775 and/or the corresponding arm 714 can also be configured based on the shape set of the arms.

Like deflectable projections 670, 670′, and 670″ described above, secondary arms 775 and 775′ can provide improved visualization of tissue capture for shunt devices 700 and 700′ in situations where the location of shunt devices 700 and 700′, or a portion of shunt devices 700 and 700′, such as ones of arms 714 and 714′, is difficult to determine from an isolated visual marker. This is because secondary arms 775 and 775′ provide for visualization of relative movement compared to a non-deflecting arm. Accordingly, shunt devices 700 and 700′ including secondary arms 775 and 775′ have improved tissue capture characteristics compared to traditional shunt devices.

### Shunt Devices 800, 800′, and 800″ (FIGS. 20A-23B)

FIG. 20A is a side view of shunt device 800 including split arm portions 880 and showing interlaced arms on a first side of shunt device 800. FIG. 20B is a top view of shunt device 800 including split arm portions 880. FIG. 20C is a flattened view of shunt device 800 including split arm portions 880. FIGS. 20A-20C will be described together. As illustrated in FIGS. 20A-20C, shunt device 800 includes body 802, which is formed of struts 804 and openings 806. Body 802 includes central flow tube 810, flow path 812, and arms 814. Shunt device 800 also includes tissue capture features 816 and radiopaque markers 818. Central flow tube 810 has side portions 820 (including side portion 820A and side portion 820B), end portions 822 (including end portion 822A and end portion 822B), first axial end 824, and second axial end 826. Arms 814 include distal arms 830 (including distal arm 830A and distal arm 830B) and proximal arms 832 (including proximal arm 832A and proximal arm 832B). Distal arms 830 have terminal ends 834 (including terminal end 834A and terminal end 834B). Proximal arms 832 have terminal ends 836 (including terminal end 836A and terminal end 836B). As is further illustrated in FIGS. 20A-20C, tissue capture features 816 include split arm portions 880 (including split arm portions 880A, 880B, and 880C), each of which includes a respective attachment end 882 (including attachment end 882A, 882B, and 882C) and terminal end 884 (including terminal end 884A, 884B, and 884C). FIG. 20A further shows horizontal reference plane HP, perpendicular reference axis RA, central axis CA, tilt angle θ, first angle α, and second angle β. FIG. 20C shows a flattened view of shunt device 800 as though shunt device 800 has been separated or cut along a cut line (not shown) drawn longitudinally through distal arm 830B, through end portion 822B, and longitudinally through proximal arm 832B.

Shunt device 800 has a generally similar structure and design to shunt devices 100 and 100′ described above in reference to FIGS. 3A-5, and shunt device 800 includes split arm portions 880. Split arm portions 880 are described here with respect to shunt device 800 but can also be included on any of the examples of a shunt device descried herewith, including shunt devices 100, 100′, 400, 500, 500′, 600, 600′, 600″, 700, 700′, 800′, 800″, 900, and 900′.

Split arm portions 880 are another form of tissue capture features 816. Split arm portions 880 separate portions of at least one of arms 814. That is, at least one of arms 814 is formed of at least two separate split arm portions 880, rather than a single arm portion. In some examples, at least one of arms 814 includes two split arm portions 880. In some examples, at least one of arms 814 includes three or more split arm portions 880. In some examples, one or more of distal arm 830 includes split arm portions 880. In the example shown in FIGS. 20A-20C, distal arm 830A includes three split arm portions 880A, 880B, and 880C. In other examples, one or more of proximal arms 832 includes split arm portions. In yet other examples, multiple of arms 814 can include split arm portions 880. Generally, any, some, or all of arms 814 can include split arm portions 880. Ones of arms 814 that include split arm portions 880 can be based at least in part on anatomical considerations, e.g., for avoiding interfering with or occluding narrow chambers or vessels in a heart, and on technical considerations relating to which arms are actuated manually during a delivery procedure of shunt device 800.

Split arm portion 880A is a first split arm portion, split arm portion 880B is a second split arm portion, and split arm portion 880C is a third split arm portion. Split arm portions 880 are connected to and extend outward from central flow tube 810 at respective attachment ends 882. Split arm portion 880A is attached to central flow tube 810 at attachment end 882A, split arm portion 880B is attached to central flow tube 810 at attachment end 882B, and split arm portion 880C is attached to central flow tube 810 at attachment end 882C. More specifically, split arm portions 880 are connected to one of the opposed pair of end portions 822 of central flow tube 810. Each split arm portion extends from the respective attachment end 882 to a respective terminal end 884. Split arm portion 880A extends from attachment end 882A to terminal end 884A, split arm portion 880B extends from attachment end 882B to terminal end 884B, and split arm portion 880C extends from attachment end 882C to terminal end 884C. Terminal ends 884 of split arm portions 880 make up a respective terminal end 834, 836 of the corresponding one of arms 814 that includes split arm portions 880. Accordingly, in the example shown in FIGS. 20A-20C, terminal ends 884A, 884B, and 884C together represent terminal end 834A of distal arm 830A.

Split arm portion 880A extends from central flow tube 810 in a first radial direction with respect to a circumference of central flow tube 810. Split arm portion 880B extends from central flow tube 810 in a second radial direction with respect to the circumference of central flow tube 810. Split arm portion 880C extends from central flow tube 810 in a third radial direction with respect to the circumference of central flow tube 810. Split arm portion 880C can originate along the one of end portions 822 at a location between split arm portions 880A and 880B. Each of split arm portions 880 can be fully separated from each other (e.g., as shown in FIGS. 20A-21B) or one or more of split arm portions 880 can be attached together (e.g., as shown in FIG. 22).

In some examples, e.g., as shown in FIGS. 20A-20C, split arm portion 880C can include a sensor attachment portion (e.g., sensor attachment portion 952 as described below with respect to FIGS. 24A-25C) for attaching to a sensor. In such examples, split arm portion 880C including the sensor attachment portion can be angled away from horizontal reference plane HP, whereas split arm portions 880A and 880B can curl or arc toward horizontal reference plane HP. In general, split arm portions 880 of one of arms 814 can encompass a similar amount of space and extend in a similar path shape as others of arms 814 that do not include split arm portions 880. However, in some examples, one of arms 814 that includes split arm portions 880 can be wider laterally than others of arms 814 due to the spread of split arm portions 880.

In general, the physical dimensions of split arm portions 880 can be selected to avoid interaction between split arm portions 880 and nearby anatomical features when shunt device 800 is implanted in a puncture in a tissue wall. For example, split arm portions 880 can be sized to avoid interaction with a mitral valve in a left atrium of a heart. The physical dimensions of split arm portions 880 can also be selected based on fatigue and strain considerations.

As is most easily seen in FIG. 20A, shunt device 800 also includes interlaced arms. One or more of arms 814 can be configured to interlace with a corresponding one of arms 814 that is on an opposite end (first axial end 824 or second axial end 826) of shunt device 800 directly across horizontal reference plane HP. In other words, clamping pairs of arms 814 can be interlaced. For example, distal arm 830A can be interlaced with proximal arm 832A, and/or distal arm 830B can be interlaced with proximal arm 832B. Clamping pairs of arms 814 can be interlaced when shunt device 800 is in a relaxed state, such as when shunt device 800 is released from a delivery catheter and is not implanted in a puncture in a tissue wall or when shunt device 800 is at least partially displaced from the puncture. Clamping pairs of arms 814 are not interlaced within the delivery catheter because arms 814 of shunt device 800 are in a straightened or compressed configuration in the delivery catheter.

As shown in FIG. 20A, split arm portions 880A and 880B of distal arm 830A are interlaced with proximal arm 832A. That is, proximal arm 832A crosses horizontal reference plane HP and crosses between split arm portions 880A and 880B in the direction of first axial end 824. Accordingly, split arm portions 880A and 880B are configured to be interlaced with proximal arm 832A on a first side of shunt device 800 that includes first axial end 824. As will be described in greater detail with reference to FIGS. 21A-21B, ones of arms 814 can alternatively be interlaced on a second side of shunt device 800 that includes second axial end 826.

Interlacing of clamping pairs of arms 814 can be accomplished with a set shape of the arms. For example, shunt device 800 can be formed of a shape-memory material, such as nitinol (a nickel titanium alloy), and arms 814 can have a set shape that causes interlaced ones of arms 814 to return to the interlaced state when shunt device 800 is in a relaxed state. Based on the set shape, one of arms 814 can be configured to bend in a first axial direction with respect to central axis CA toward the corresponding one of arms 814 in the clamping pair. For example, FIG. 20A shows proximal arm 832A bent in the first axial direction toward distal arm 830A and split arm portions 880. In some examples, the corresponding one of arms 814 in the clamping pair can also be configured to bend in a second axial direction with respect to central axis CA that is opposite the first direction. Regardless of whether one or both arms 814 in the clamping pair are bent, the set shapes can permit the arms to cross and interlace.

In the interlaced clamping pair of arms 814, the one or more bent arms can be relatively flexible and compliant so that after the arms 814 are released from a delivery catheter the bent arm will not apply significant pressure to a tissue wall and cause the tissue to tent or deform. If the bent arm is too stiff, the tissue may deform such that it will look like there is interlacing when there is actually tissue between the clamping pair of arms.

Radiopaque markers 818 can be included on shunt device 800. Radiopaque markers 818 are structures that are dense and resist the passage of X-rays to permit visualization with radiographic imaging. For example, radiopaque markers 818 can be attached or stamped at terminal ends 884 of split arm portions 880. In some examples, radiopaque markers 818 can also be attached at multiple ones of terminal ends 834, 836 and to other tissue capture features 816. For example, FIGS. 20A-20C show radiopaque markers 818 at terminal ends 834A, 834B, and 836B and associated with split arm portions 880. In other examples, radiopaque markers 818 can be attached to any suitable portion or portions of shunt device 800, such as other locations on arms 814 or other locations on tissue capture features 816. Moreover, shunt device 800 can include any suitable number of radiopaque markers 818. In yet other examples, shunt device 800 may not include any radiopaque markers 818. Radiopaque markers 818 can be shaped and sized to fit on the portions of shunt device 800 to which they are attached. In other examples, radiopaque markers 818 can be thickened regions of struts 804 rather than separate pieces that are attached. For example, shunt device 800 can be formed of nitinol (a nickel titanium alloy) and radiopaque markers 818 can be relatively thickened regions of nitinol.

FIG. 21A is a side view of shunt device 800′ including split arm portions 880′ and showing interlaced arms on a second side of shunt device 800′. FIG. 21B is a top view of shunt device 800′ including split arm portions 880′ and showing the interlaced arms on the second side of shunt device 800′. FIGS. 21A-21B will be described together. As illustrated in FIGS. 21A-21B, shunt device 800′ includes body 802′, which is formed of struts 804′ and openings 806′. Body 802′ includes central flow tube 810′, flow path 812′, and arms 814′. Shunt device 800′ also includes tissue capture features 816′ and radiopaque markers 818′. Central flow tube 810′ has side portions 820′ (including side portion 820A′ and side portion 820B′), end portions 822′ (including end portion 822A′ and end portion 822B′), first axial end 824′, and second axial end 826′. Arms 814′ include distal arms 830′ (including distal arm 830A′ and distal arm 830B′) and proximal arms 832′ (including proximal arm 832A′ and proximal arm 832B′). Distal arms 830′ have terminal ends 834′ (including terminal end 834A′ and terminal end 834B′). Proximal arms 832′ have terminal ends 836′ (including terminal end 836A′ and terminal end 836B′). As is further illustrated in FIGS. 21A-21B, tissue capture features 816′ include split arm portions 880′ (including split arm portions 880A′, 880B′, and 880C′). FIG. 21A further shows horizontal reference plane HP, perpendicular reference axis RA, central axis CA, tilt angle θ, first angle α, and second angle β.

Shunt device 800′ has a generally similar structure and design to shunt device 800 described above, except shunt device 800′ includes split arm portions 880′ instead of split arm portions 880. Compared to split arm portions 880 as shown in FIGS. 20A-20C, split arm portions 880′ are configured to interlace with proximal arm 832A′ on a second side of shunt device 800′ that includes second axial end 826. That is, split arm portions 880A′ and 880B′, rather than proximal arm 832A′, cross horizontal reference plane HP in the direction of second axial end 826′ so that proximal arm 832A′ crosses between split arm portions 880A′ and 880B′ on the second side of shunt device 800′.

FIG. 22 is a flattened view of shunt device 800″ including split arm portions 880″. As illustrated in FIG. 22, shunt device 800″ includes body 802″, which is formed of struts 804″ and openings 806″. Body 802″ includes central flow tube 810″, and arms 814″. Shunt device 800″ also includes tissue capture features 816″ and radiopaque markers 818″. Central flow tube 810″ has side portions 820″ (including side portion 820A″ and side portion 820B″), end portions 822″ (including end portion 822A″ and end portion 822B″), first axial end 824″, and second axial end 826″. Arms 814″ include distal arms 830″ (including distal arm 830A″ and distal arm 830B″) and proximal arms 832″ (including proximal arm 832A″ and proximal arm 832B″). Distal arms 830″ have terminal ends 834″ (including terminal end 834A″ and terminal end 834B″). Proximal arms 832″ have terminal ends 836″ (including terminal end 836A″ and terminal end 836B″). As is further illustrated in FIG. 22, tissue capture features 816″ include split arm portions 880″ (including split arm portions 880A″, 880B″, and 880C″). FIG. 22 shows a flattened view of shunt device 800″ as though shunt device 800″ has been separated or cut along a cut line (not shown) drawn longitudinally through distal arm 830B″, through end portion 822B″, and longitudinally through proximal arm 832B″.

Shunt device 800″ has a generally similar structure and design to shunt device 800 described above, except shunt device 800″ includes split arm portions 880″ instead of split arm portions 880. Compared to split arm portions 880 as shown in FIGS. 20A-20C, split arm portions 880″ of distal arm 830A″ form a loop or horseshoe shape rather than being fully separated. That is, split arm portions 880A″ and 880B″ are connected in a loop that surrounds split arm portion 880C″. In an interlaced configuration, proximal arm 832A″ can cross split arm portions 880A″ and 880B″ within a perimeter of the loop. It will also be appreciated that shunt device 800″ is structurally similar to shunt device 700′ (as shown in FIG. 18) but split arm portions 880″ may not have different compliances (compared to secondary arm 775′ and the associated distal arm 730A′, as shown in FIG. 18) and may be in an interlaced configuration.

FIG. 23A is a schematic view of shunt device 800 including split arm portions 880 in an interlaced state. FIG. 23B is a schematic view of shunt device 800 including split arm portions 880 in a separated state. FIGS. 23A-23B show shunt device 800, arms 814 (including distal arms 830A and 830B and proximal arms 832A and 832B), and split arm portions 880. FIGS. 23A-23B also show tissue wall TW. It should be noted that both FIGS. 23A and 23B show shunt device 800 with one of split arm portions 880 configured to attach to a sensor device but with the sensor device omitted for ease of illustrating the position of split arm portions 880.

Shunt devices 800, 800′, and 800″ can be delivered into a human body using, for examples delivery catheter 200 (as shown in FIGS. 6-7B) and according to, for example, method 300 described above in reference to FIGS. 8A-9R. The operation of shunt devices 800, 800′, and 800″ will be described with reference to FIGS. 20A-23B together. For ease of discussion, functionality will be described with respect to shunt device 800 and its components; however, it should be understood that the functionality described herein is applicable to each of shunt devices 800, 800′, and 800″, except where a difference is indicated.

FIG. 23A shows shunt device 800 in a relaxed state, such as when shunt device 800 is released from a delivery catheter and is not implanted in a puncture in a tissue wall or when shunt device 800 is at least partially displaced from the puncture. As shown in FIG. 23A, split arm portions 880 of distal arm 830A are interlaced with proximal arm 832A because tissue wall TW is not captured between distal arm 830A and proximal arm 832A.

In contrast, FIG. 23B shows shunt device 800 and split arm portions 880 in a separated state because tissue wall TW is captured between distal arm 830A and proximal arm 832A. Distal arms 830A and 830B are released first from a delivery catheter during an implantation procedure, so when shunt device 800 is properly seated, distal arms 830A and 830B will be on a same side of tissue wall TW. For example, distal arms 830A and 830B can be on a left atrial side of tissue wall TW when shunt device 800 is implanted in a puncture in a tissue wall between a left atrium and a coronary sinus of a heart. If shunt device 800 remains properly seated in the puncture as proximal arms 832A and 832B are released from the delivery catheter, proximal arms 832A and 832B will be on an opposite side of tissue wall TW (e.g., a coronary sinus side). Accordingly, tissue wall TW will be captured between clamping pairs of distal arm 830A with proximal arm 832A and distal arm 830B with proximal arm 832B. When there is tissue capture, split arm portions 880 of distal arm 830A will no longer be interlaced with proximal arm 832A because they are maintained apart by tissue wall TW. That is, split arm portions 880 of distal arm 830A will be separated from the corresponding proximal arm 832A such that there is a gap between them that spans a section of tissue wall TW. Separation of interlaced arms, such as split arm portions 880 and proximal arm 832A, can be detected with fluoroscopy or other real-time imaging techniques. For example, a physician can track separation of split arm portions 880 from proximal arm 832A, or the corresponding one of arms 814 with which split arm portions 880 were interlaced, by visualizing a change in the location of radiopaque markers 818 with fluoroscopic imaging. When there is no tissue captured between the clamping pair of arms 814, the interlaced configuration can also be visualized based on the location of radiopaque markets 818 in close proximity to each other.

Including split arm portions 880, 880′, and 880″ on shunt devices 800, 800′, and 800″ allows shunt devices 800, 800′, and 800″ to have a greater variety of design configurations so that shunt devices 800, 800′, and 800″ may be more broadly applicable for various cardiovascular anatomies or medical conditions. In particular, including split arm portions 880, 880′, and 880″ permits interlacing as well as having a separate arm portion for attaching a sensor. Interlacing of clamping pairs of arms 814, 814′, and 814″ provides an additional option for confirming tissue capture and proper seating of shunt devices 800, 800′, and 800″ via visualization of the change between the interlaced state and the separated state. This can be beneficial on sides of shunt devices 800, 800′, and 800″ where it is more difficult to know if there is tissue capture and proper seating. Accordingly, shunt devices 800, 800′, and 800″ including split arm portions 880, 880′, and 880″ have improved tissue capture characteristics compared to traditional shunt devices.

### Shunt Devices 900 and 900′ (FIGS. 24A-26B)

FIG. 24A is a side view of shunt device 900 including sensor 950. FIG. 24B is a flattened view of shunt device 900 with sensor 950 removed. FIGS. 25A-25C are enlarged views showing details of sensor 950 and sensor attachment portion 952 of shunt device 900. FIGS. 24A-25C will be described together. As illustrated in FIGS. 24A-24B, shunt device 900 includes body 902, which is formed of struts 904 and openings 906. Body 902 includes central flow tube 910, flow path 912, and arms 914. Shunt device 900 also includes tissue capture features 916 and radiopaque markers 918. Central flow tube 910 has side portions 920 (including side portion 920A and side portion 920B), end portions 922 (including end portion 922A and end portion 922B), first axial end 924, and second axial end 926. Arms 914 include distal arms 930 (including distal arm 930A and distal arm 930B) and proximal arms 932 (including proximal arm 932A and proximal arm 932B). Distal arms 930 have terminal ends 934 (including terminal end 934A and terminal end 934B). Proximal arms 932 have terminal ends 936 (including terminal end 936A and terminal end 936B). Shunt device 900 further includes sensor 950 and sensor attachment portion 952. Sensor 950 includes sensor element 953, housing 954, one or more mating features 955 (as shown in FIGS. 25A and 25C), and binding 956. Sensor attachment portion 952 includes mating interface 957 (as shown in FIGS. 24B, 25A, and 25C) and one or more mating features 958 (as shown in FIGS. 24B, 25A, and 25C). Housing 954 includes central cavity 959 (as shown in FIGS. 24A and 25A-25C). FIG. 24A further shows horizontal reference plane HP, perpendicular reference axis RA, central axis CA, tilt angle θ, first angle α, and second angle β. FIG. 24B shows a flattened view of shunt device 900 as though shunt device 900 has been separated or cut along a cut line (not shown) drawn longitudinally through distal arm 930B, through end portion 922B, and longitudinally through proximal arm 932B.

Shunt device 900 has a generally similar structure and design to shunt devices 100 and 100′ described above in reference to FIGS. 3A-5. Details of sensor 950 and sensor attachment portion 952 are described here with respect to shunt device 900 but can also be included on any of the examples of a shunt device descried herewith, including shunt devices 100, 100′, 400, 500, 500′, 600, 600′, 600″, 700, 700′, 800, 800′, 800″, and 900′.

Sensor 950 is any suitable sensor device attached to shunt device 900 for sensing a parameter in a chamber or vessel of a heart. In some examples, sensor 950 is configured to sense a parameter in a left atrium of the heart. Alternatively, sensor 950 can be configured to sense a parameter in any other chamber or vessel of the heart, such as a coronary sinus or a right atrium, depending on the site and orientation where shunt device 900 is implanted. In one example, sensor 950 can be a pressure sensor. Although shunt device 900 is illustrated in FIG. 24A as including a single sensor 950, it should be understood that other examples can include multiple sensors 950 attached to shunt device 900, e.g., on different arms 914.

Sensor 950 includes sensor element 953, which is a main sensing component of sensor 950. Sensor element 953 can contain internal components for sensing the parameter in the chamber or vessel and wirelessly communicating a signal representing the sensed data to a receiver located externally to the body. In some examples, sensor 950 includes a single type of sensor element 953. In other examples, sensor 950 can include multiple sensor elements 953, such as for sensing multiple parameters.

Sensor 950 also includes housing 954 to contain sensor element 953. Housing 954 defines central cavity 959 within which sensor element 953 can be positioned. Housing 954 can be generally cylindrical in shape to surround sensor element 953. Housing 954 holds sensor element 953 in position within central cavity 959 once sensor element 953 is inserted or positioned in central cavity 959. Accordingly, in some examples, central cavity 959 can be shaped so that the interior of housing 954 closely fits to an exterior of sensor element 953 (i.e., so that sensor element 953 is held snugly rather than loosely within central cavity 959). Housing 954 can be formed of any suitable material for use in a human body. In some examples, housing 954 is formed of a thermoplastic material. In some examples, housing 954 is formed of a polyetheretherketone (PEEK). As will be described in greater detail below with respect to mating features 955 and 958, housing 954 can include a groove or notch that aligns with a portion of one of arms 914 of shunt device 900. As shown in FIG. 24A, sensor 950 also includes binding 956, which wraps around housing 954 to secure housing 954 (and sensor element 953 therein) to one of arms 914. For example, binding 956 can be a suture thread or wire.

Sensor attachment portion 952 is a structural region of one of arms 914 for attaching to sensor 950. The one of arms 914 that includes sensor attachment portion 952 will be referred to herein as a “sensor arm 914.” In some examples, sensor attachment portion 952 can be part of one of distal arms 930. As illustrated in FIGS. 24A-24B, sensor attachment portion 952 can be part of distal arm 430A. In other examples, sensor attachment portion 952 can be part of one of proximal arms 932. In some examples, sensor attachment portion 952 can be part of a split arm portion (e.g., split arm portions 880, 880′, and 880″, as shown in FIGS. 20A-22) if one of arms 914 includes split arm portions. In other examples, sensor attachment portion 952 can be a part of a secondary arm associated with one of arms 914 (e.g., secondary arm 775′ as shown in FIG. 22). Generally, sensor attachment portion 952 can be included on any, some, or all of arms 914.

Sensor attachment portion 952 can be angled to hold sensor 950 at an optimal angle and location in a chamber or vessel of a heart for both safety and signal integrity. For example, sensor attachment portion 952 can be angled to prevent sensor 950 from contacting nearby tissue walls to avoid damaging the sensor or tissue and to avoid interference with sensor readings. When shunt device 900 is implanted in the tissue wall between a left atrium and a coronary sinus of the heart, sensor 950 can be positioned in the left atrium, and sensor attachment portion 952 can be angled to avoid both a mitral valve and a high pressure, high flow mitral regurgitation jet, exposure to which could cause high strain and fatigue to sensor 950 and sensor attachment portion 952. In general, sensor attachment portion 952 is angled away from horizontal reference plane HP such that sensor 950 will be angled away from a tissue wall when shunt device 900 is inserted into a puncture in the tissue wall. In some examples, sensor attachment portion 952 forms an angle between zero (0°) and forty-five degrees (45°) with perpendicular reference axis RA. In some examples, sensor attachment portion 952 forms an angle of about fifteen to sixteen degrees (15°-16°) with perpendicular reference axis RA.

Sensor attachment portion 952 includes mating interface 957. Mating interface 957 is a part of sensor attachment portion 952 that attaches to and interlocks with sensor 950. Mating interface 957 includes mating features 958. Mating features 958 of sensor attachment portion 952 are complimentary to mating features 955 of sensor 950. Mating features 958 can be slots or openings in the sensor arm 914 that are sized to fit mating features 955. For example, as shown in FIGS. 24B, 25A, and 25C, the sensor arm 914 can include two slots. In other examples, the sensor arm 914 can include any number of slots or mating features 958. Mating features 955 can be raised portions or bumps that are configured to fit into the slots (mating features 958) to interlock sensor 950 to the sensor arm 914. In some examples, mating features 955 can be a part of an exterior surface of housing 954. In some examples, the raised portions are within a groove or notch on the exterior surface of housing 954. As shown in FIGS. 25A and 25C, housing 954 can include two raised portions to correspond to the two slots on the sensor arm 914. In other examples, sensor 950 can include any number of raised portions or mating features 955 to correspond to a number of mating features 958.

Radiopaque markers 918 can be included on shunt device 900. Radiopaque markers 918 are structures that are dense and resist the passage of X-rays to permit visualization with radiographic imaging. In some examples, radiopaque markers 918 can be attached at multiple ones of terminal ends 934, 936 and to one of tissue capture features 916. For example, FIGS. 24A-24B show radiopaque markers 918 at terminal ends 834B and 836B and associated with tissue capture features 916. In other examples, radiopaque markers 918 can be attached to any suitable portion or portions of shunt device 900, such as other locations on arms 914 or other locations on tissue capture features 916. Moreover, shunt device 900 can include any suitable number of radiopaque markers 918. In yet other examples, shunt device 900 may not include any radiopaque markers 918. Radiopaque markers 918 can be shaped and sized to fit on the portions of shunt device 900 to which they are attached. In other examples, radiopaque markers 918 can be thickened regions of struts 904 rather than separate pieces that are attached. For example, shunt device 900 can be formed of nitinol (a nickel titanium alloy) and radiopaque markers 918 can be relatively thickened regions of nitinol.

FIG. 26A is a side view of shunt device 900′ including sensor 950′. FIG. 26B is a flattened view of shunt device 900′ with sensor 950′ removed. FIGS. 26A-26B will be described together. As illustrated in FIGS. 26A-26B, shunt device 900′ includes body 902′, which is formed of struts 904′ and openings 906′. Body 902′ includes central flow tube 910′, flow path 912′, and arms 914′. Shunt device 900′ also includes tissue capture features 916′ and radiopaque markers 918′. Central flow tube 910′ has side portions 920′ (including side portion 920A′ and side portion 920B′), end portions 922′ (including end portion 922A′ and end portion 922B′), first axial end 924′, and second axial end 926′. Arms 914′ include distal arms 930′ (including distal arm 930A′ and distal arm 930B′) and proximal arms 932′ (including proximal arm 932A′ and proximal arm 932B′). Distal arms 930′ have terminal ends 934′ (including terminal end 934A′ and terminal end 934B′). Proximal arms 932′ have terminal ends 936′ (including terminal end 936A′ and terminal end 936B′). Shunt device 900′ further includes sensor 950′ and sensor attachment portion 952′. Sensor 950′ includes sensor element 953′, housing 954′, one or more mating features 955′ (as indicated with the dashed line), and binding 956′. Sensor attachment portion 952′ includes mating interface 957′ and one or more mating features 958′. Housing 954′ includes central cavity 959′. As is further illustrated in FIGS. 26A-26B, tissue capture features 916′ include split arm portions 980′ (including split arm portions 980A′, 980B′, and 980C′). FIG. 26A further shows horizontal reference plane HP, perpendicular reference axis RA, central axis CA, tilt angle θ, first angle α, and second angle β. FIG. 26B shows a flattened view of shunt device 900′ as though shunt device 900′ has been separated or cut along a cut line (not shown) drawn longitudinally through distal arm 930B′, through end portion 922B′, and longitudinally through proximal arm 932B′.

Shunt device 900′ has a generally similar structure and design to shunt device 900 described above, except shunt device 900′ includes sensor 950′ and sensor attachment portion 952′ instead of sensor 950 and sensor attachment portion 952. In this example, sensor attachment portion 952′ is part of one of split arm portions 980′ that is similar to split arm portions 880, 880′, and 880″ shown in FIGS. 20A-22. Specifically, sensor attachment portion 952′ is part of split arm portion 980C′. Sensor attachment portion 952′ on split arm portion 980C′ is between split arm portion 980A′ and split arm portion 980B′. As shown in FIGS. 26A-26B, split arm portions 980A′ and 980B′ are fully separated.

Shunt devices 900 and 900′ can be delivered into a human body using, for example, delivery catheter 200 (as shown in FIGS. 6-7B) and according to, for example, method 300 described above in reference to FIGS. 8A-9R. The operation of shunt devices 900 and 900′ will be described with reference to FIGS. 24A-26B together. Once shunt devices 900 and 900′ are inserted into a puncture in a tissue wall of a heart, sensors 950 and 950′ will measure parameters, such as pressures, in a chamber or vessel of the heart in which sensors 950 and 950′ are positioned. These measurements can be communicated to an external receiver where a physician or other user may review them to obtain information about the conditions in the heart near shunt devices 900 and 900′.

Other shunt devices that have sensors attached by an added or non-integral connection mechanism, such as by a tether or other mechanism, can experience significant fatigue at the connection mechanism or on the sensor if the sensor is not anchored tightly. Sensor attachment portions 952 and 952′ permit respective sensors 950 and 950′ to be interlocked with the corresponding device arm, which can reduce fatigue.

Additionally, including mating features 955 and 955′ on respective housings 954 and 954′, which further include internal cavities with snap mechanisms to hold sensors 950 and 950′, allow housings 954 and 954′ to be pre-attached to respective sensor attachment portions 952 and 952′. In this way, sensors 950 and 950′ can be readily added to shunt devices 900 and 900′. Some examples of sensors 950 and 950′ must be maintained within very tight temperature control specifications otherwise they can become damaged, but generally shunt devices and delivery catheters do not have such requirements and would also be difficult to maintain such tight temperature control for. In such examples, sensors 950 and 950′ can be kept separately from shunt devices 900 and 900′ and the delivery catheter in a temperature-controlled environment and then attached within respective housings 954 and 954′ at bedside immediately prior to an implantation procedure.

Moreover, sensor attachment portions 952 and 952′ are compatible with several variations of tissue capture features (e.g., tissue capture features 116, 116′, 416, 516, 516′, 616, 616′, 616″, 716, 716′, 816, 816′, 816″, 916, and 916′ according to techniques described herein), so shunt devices 900 and 900′ can combine additional sensing capabilities with improved tissue capture and visualization.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Discussion of Possible Examples

The following are non-exclusive descriptions of possible examples of the present invention.

A shunt device is configured to be inserted into a puncture in a tissue wall that defines a horizontal reference plane. The shunt device includes a shunt body formed of a plurality of struts. The shunt body includes a central flow tube extending from a first axial end to a second axial end and defining a central axis therethrough that is angled from a reference axis extending perpendicular through the horizontal reference plane; a flow path extending through the central flow tube; and a plurality of arms extending outward from the central flow tube and configured to secure the shunt device to the tissue wall. Each arm of the plurality of arms extends from the central flow tube to a terminal end. The plurality of arms includes a first distal arm and a second distal arm attached to the first axial end of the central flow tube and a first proximal arm and a second proximal arm attached to the second axial end of the central flow tube. At least one of the plurality of arms includes a lengthened portion adjacent a respective terminal end.

The shunt device of the preceding paragraph can optionally include, additionally and/or alternatively, any one or more of the following features, configurations, and/or additional components:

The lengthened portion can extend generally parallel to the horizontal reference plane.

The lengthened portion can be generally straight.

The lengthened portion can be narrower than a respective proximal portion of the at least one of the plurality of arms adjacent to the central flow tube.

The lengthened portion can further include a tapered portion adjacent the respective proximal portion.

The tapered portion can be narrower towards the respective terminal end and wider towards the respective proximal portion.

The at least one of the plurality of arms that includes the lengthened portion can be deflectable along a deflection arc away from the horizontal reference plane when a delivery catheter for the shunt device is pulled back during a shunt device implantation process.

A central angle of the deflection arc can be between zero and ninety degrees.

Each of the distal arms can form a pair of arms with a corresponding one of the proximal arms to secure the shunt device to the tissue wall, and the at least one of the plurality of arms that includes the lengthened portion can be longer than a corresponding one of the plurality of arms with which it forms the pair.

The at least one of the plurality of arms that includes the lengthened portion can include a radiopaque marker at the respective terminal end.

The shunt device can be configured to be inserted into the puncture in the tissue wall such that the first axial end of the central flow tube faces a left atrium of a heart and the second axial end of the central flow tube faces a coronary sinus of the heart, and the at least one of the plurality of arms that includes the lengthened portion can be one of the distal arms.

The first distal arm can form a first angle with the central axis of the central flow tube, the first angle being less than ninety degrees; the second distal arm can form a second angle with the central axis of the central flow tube, the second angle being greater than ninety degrees;

and the at least one of the plurality of arms that includes the lengthened portion can be the second distal arm.

The shunt device can be sterilized.

The shunt device can be formed of a shape-memory material.

The shape-memory material can be nitinol.

The shunt device can further include a sensor attached to one of the plurality of arms of the shunt body at a mating interface such that the sensor and the one of the plurality of arms are interconnected.

The sensor can include a pressure sensor.

A shunt device is configured to be inserted into a puncture in a tissue wall that defines a horizontal reference plane. The shunt device includes a shunt body formed of a plurality of struts. The shunt body includes a central flow tube extending from a first axial end to a second axial end and defined by an opposed pair of side portions that extend laterally between an opposed pair of end portions; a flow path extending through the central flow tube; and a plurality of arms extending outward from the central flow tube and configured to secure the shunt device to the tissue wall. The plurality of arms includes a first distal arm and a second distal arm attached to the first axial end of the central flow tube and a first proximal arm and a second proximal arm attached to the second axial end of the central flow tube. The shunt device further includes one or more tabs extending outward from the central flow tube and configured to prevent the shunt device from displacing through the puncture.

The shunt device of the preceding paragraph can optionally include, additionally and/or alternatively, any one or more of the following features, configurations, and/or additional components:

The one or more tabs can be attached to the first axial end of the central flow tube.

The one or more tabs can be attached to the second axial end of the central flow tube.

The shunt device can be configured to be inserted into the puncture in the tissue wall such that the first axial end of the central flow tube faces a left atrium of a heart and the second axial end of the central flow tube faces a coronary sinus of the heart, and one or more tabs can be attached to the first axial end of the central flow tube such that the one or more tabs contact a left atrial side of the tissue wall.

The one or more tabs can be attached to a side portion of the opposed pair of side portions of the central flow tube.

The one or more tabs can include a first tab attached to a first side portion of the opposed pair of side portions of the central flow tube and a second tab attached to a second side portion of the opposed pair of side portions of the central flow tube.

Each of the one or more tabs can have a shorter length than the distal arms and the proximal arms.

At least one tab of the one or more tabs can include a radiopaque marker.

The one or more tabs can be formed of a same material as the shunt body.

The one or more tabs can be part of a single laser cut pattern for the shunt device, and the shunt device including the one or more tabs can be a monolithic structure.

The one or more tabs can be welded to the central flow tube.

The one or more tabs can be formed of a different material from the shunt body, and the one or more tabs can be welded to the central flow tube.

The shunt device can be sterilized.

The shunt device can be formed of a shape-memory material.

The shape-memory material can be nitinol.

The shunt device can further include a sensor attached to one of the plurality of arms of the shunt body at a mating interface such that the sensor and the one of the plurality of arms are interconnected.

The sensor can include a pressure sensor.

A shunt device is configured to be inserted into a puncture in a tissue wall that defines a horizontal reference plane. The shunt device includes a shunt body formed of a plurality of struts. The shunt body includes a central flow tube extending from a first axial end to a second axial end and defined by an opposed pair of side portions that extend laterally between an opposed pair of end portions; a flow path extending through the central flow tube; and a plurality of arms extending outward from the central flow tube and configured to secure the shunt device to the tissue wall. The plurality of arms includes a first distal arm and a second distal arm attached to the first axial end of the central flow tube and a first proximal arm and a second proximal arm attached to the second axial end of the central flow tube. The shunt device further includes a deflectable projection connected to at least one of the plurality of arms.

The shunt device of the preceding paragraph can optionally include, additionally and/or alternatively, any one or more of the following features, configurations, and/or additional components:

The deflectable projection can be connected to the at least one of the plurality of arms at a location distal to the central flow tube and extend radially inward toward the central flow tube.

The deflectable projection can be connected to the at least one of the plurality of arms at a location proximal to the central flow tube and extend radially outward toward a respective terminal end of the at least one of the plurality of arms.

The deflectable projection can be more compliant than the at least one of the plurality of arms.

The deflectable projection and the at least one of the plurality of arms can be biased away from each other such that there is a gap between the deflectable projection and the at least one of the plurality of arms when the shunt device is in a relaxed state.

The deflectable projection can be configured to deflect toward the at least one of the plurality of arms such that the gap is reduced when the shunt device is inserted in the puncture in the tissue wall and there is tissue capture by the at least one of the plurality of arms.

The deflectable projection can extend within a perimeter of the at least one of the plurality of arms.

The deflectable projection can be formed of thinner struts than the at least one of the plurality of arms.

The deflectable projection can include several hairpin curved portions.

The deflectable projection can include a radiopaque marker.

Deflection of the deflectable projection can be detectable with fluoroscopy.

The deflectable projection can be formed of a same material as the shunt body.

The deflectable projection can be part of a single laser cut pattern for the shunt device, and the shunt device including the deflectable projection can be a monolithic structure.

The deflectable projection can be welded to the at least one of the plurality of arms.

The deflectable projection can be formed of a different material from the shunt body, and the deflectable projection can be welded to the at least one of the plurality of arms.

The shunt device can be sterilized.

The shunt device can be formed of a shape-memory material.

The shape-memory material can be nitinol.

The shunt device can further include a sensor attached to one of the plurality of arms of the shunt body at a mating interface such that the sensor and the one of the plurality of arms are interconnected.

The sensor can include a pressure sensor.

A shunt device is configured to be inserted into a puncture in a tissue wall that defines a horizontal reference plane. The shunt device includes a shunt body formed of a plurality of struts. The shunt body includes a central flow tube extending from a first axial end to a second axial end and defined by an opposed pair of side portions that extend laterally between an opposed pair of end portions; a flow path extending through the central flow tube; and a plurality of arms extending outward from the central flow tube and configured to secure the shunt device to the tissue wall. The plurality of arms includes a first distal arm and a second distal arm attached to the first axial end of the central flow tube and a first proximal arm and a second proximal arm attached to the second axial end of the central flow tube. The shunt device further includes a secondary arm associated with at least one of the plurality of arms.

The shunt device of the preceding paragraph can optionally include, additionally and/or alternatively, any one or more of the following features, configurations, and/or additional components:

The secondary arm can be connected to an end portion of the opposed pair of end portions of the central flow tube and extend radially outward with the at least one of the plurality of arms.

The secondary arm can extend within a perimeter of the at least one of the plurality of arms.

The secondary arm can be more compliant than the at least one of the plurality of arms.

The secondary arm and the at least one of the plurality of arms can be biased away from each other such that there is a gap between the secondary arm and the at least one of the plurality of arms when the shunt device is in a relaxed state; and the secondary arm can be configured to deflect toward the at least one of the plurality of arms such that the gap is reduced when the shunt device is inserted in the puncture in the tissue wall and there is tissue capture by the at least one of the plurality of arms.

The secondary arm can be less compliant than the at least one of the plurality of arms.

The secondary arm and the at least one of the plurality of arms can be biased away from each other such that there is a gap between the secondary arm and the at least one of the plurality of arms when the shunt device is in a relaxed state; and the at least one of the plurality of arms can be configured to deflect toward the secondary arm such that the gap is reduced when the shunt device is inserted in the puncture in the tissue wall and there is tissue capture by the at least one of the plurality of arms.

The shunt device can be configured to be inserted into the puncture in the tissue wall such that the first axial end of the central flow tube faces a left atrium of a heart and the second axial end of the central flow tube faces a coronary sinus of the heart; and the secondary arm can be associated with one of the distal arms.

The central flow tube can define a central axis therethrough that is angled from a reference axis extending perpendicular through the horizontal reference plane; the first distal arm can form a first angle with the central axis of the central flow tube, the first angle being less than ninety degrees; the second distal arm can form a second angle with the central axis of the central flow tube, the second angle being greater than ninety degrees; and the secondary arm can be associated with the second distal arm.

The secondary arm can include a radiopaque marker.

Deflection of the secondary arm and/or the at least one of the plurality of arms can be detectable with fluoroscopy.

The secondary arm can be formed of a same material as the shunt body.

The secondary arm can be part of a single laser cut pattern for the shunt device, and

the shunt device including the secondary arm can be a monolithic structure.

The secondary arm can be welded to the shunt body.

The secondary arm can be formed of a different material from the shunt body, and

the secondary arm can be welded to the shunt body.

The shunt device can be sterilized.

The shunt device can be formed of a shape-memory material.

The shape-memory material can be nitinol.

The shunt device can further include a sensor attached to the secondary arm at a mating interface such that the sensor and the secondary arm are interconnected.

The sensor can include a pressure sensor.

At least one of the plurality of arms can include a lengthened portion adjacent a respective terminal end.

The shunt device can further include one or more tabs extending outward from the central flow tube and configured to prevent the shunt device from displacing through the puncture.

The shunt device can further include a deflectable projection connected to at least one of the plurality of arms.

At least one of the plurality of arms can include two or more split arm portions.

A shunt device is configured to be inserted into a puncture in a tissue wall that defines a horizontal reference plane. The shunt device includes a shunt body formed of a plurality of struts. The shunt body includes a central flow tube extending from a first axial end to a second axial end and defining a central axis therethrough that is angled from a reference axis extending perpendicular through the horizontal reference plane; a flow path extending through the central flow tube; and a plurality of arms extending outward from the central flow tube and configured to secure the shunt device to the tissue wall. The plurality of arms includes a first distal arm and a second distal arm attached to the first axial end of the central flow tube and a first proximal arm and a second proximal arm attached to the second axial end of the central flow tube. At least one of the plurality of arms includes two or more split arm portions.

The shunt device of the preceding paragraph can optionally include, additionally and/or alternatively, any one or more of the following features, configurations, and/or additional components:

A first split arm portion of the two or more split arm portions can extend in a first radial direction and a second split arm portion can extend in a second radial direction from a circumference of the central flow tube.

The two or more split arm portions can further include a third split arm portion extending from the circumference of the central flow tube between the first split arm portion and the second split arm portion.

A first split arm portion of the two or more split arm portions can be horseshoe shaped such that the first split arm portion surrounds a second split arm portion.

The first distal arm can form a first angle with the central axis of the central flow tube, the first angle being less than ninety degrees; the second distal arm can form a second angle with the central axis of the central flow tube, the second angle being greater than ninety degrees; and the at least one of the plurality of arms that includes the two or more split arm portions can be the first distal arm.

At least one of the two or more split arm portions and a corresponding arm of the plurality of arms that is attached to an opposite end of the central flow tube from the two or more split arm portions can be shape set to be interlaced when the shunt device is in a relaxed state.

The at least one of the two or more split arm portions and the corresponding arm can be interlaced on a side of the shunt device that includes the first axial end of the central flow tube.

The at least one of the two or more split arm portions and the corresponding arm can be interlaced on a side of the shunt device that includes the second axial end of the central flow tube.

The at least one of the two or more split arm portions and the corresponding arm can be separated such that there is a gap between the at least one of the two or more split arm portions and the corresponding arm when the shunt device is inserted in the puncture in the tissue wall and there is tissue capture between the at least one of the two or more split arm portions and the corresponding arm. Separation of the at least one of the two or more split arm portions and the corresponding arm can be detectable with fluoroscopy.

At least one of the two or more split arm portions can include a radiopaque marker. The shunt device can be sterilized.

The shunt device can be formed of a shape-memory material.

The shape-memory material can be nitinol.

The shunt device can further include a sensor attached to one of the two or more split arm portions at a mating interface such that the sensor and the one of the two or more split arm portions are interconnected.

The sensor can include a pressure sensor.

A shunt device is configured to be inserted into a puncture in a tissue wall that defines a horizontal reference plane. The shunt device includes a shunt body formed of a plurality of struts. The shunt body includes a central flow tube extending from a first axial end to a second axial end and defining a central axis therethrough that is angled from a reference axis extending perpendicular through the horizontal reference plane; a flow path extending through the central flow tube; and a plurality of arms extending outward from the central flow tube and configured to secure the shunt device to the tissue wall. The shunt device further includes a sensor attached to one of the plurality of arms of the shunt body at a mating interface such that the sensor and the one of the plurality of arms are interconnected.

The shunt device of the preceding paragraph can optionally include, additionally and/or alternatively, any one or more of the following features, configurations, and/or additional components:

The one of the plurality of arms can include a slot at the mating interface and the sensor can include a raised portion that is configured to fit into the slot to interlock the sensor and the one of the plurality of arms at the mating interface.

The sensor can further include a housing that contains a sensor element.

The one of the plurality of arms can include a slot at the mating interface and the housing of the sensor can include a raised portion that is configured to fit into the slot to interlock the sensor and the one of the plurality of arms at the mating interface.

The housing can be cylindrical and define a central cavity, and the sensor element can be positioned in the central cavity of the housing.

The housing can be formed of a thermoplastic material.

The thermoplastic material can be a polyetheretherketone (PEEK).

The housing can be secured to the one of the plurality of arms by a binding that is wrapped around the housing and the one of the plurality of arms near the mating interface.

The one of the plurality of arms can further include split arm portions, the split arm portions including a first split arm portion that extends in a first radial direction from a circumference of the central flow tube, a second split arm portion that extends in a second radial direction from the circumference of the central flow tube, and a third split arm portion that extending from the circumference of the central flow tube between the first split arm portion and the second split arm portion; and the sensor can be attached to the third split arm portion.

The one of the plurality of arms can further include split arm portions, a first split arm portion of the split arm portions can be horseshoe shaped such that the first split arm portion surrounds a second split arm portion, and the sensor can be attached to the second split arm portion.

The plurality of arms can further include a first distal arm and a second distal arm attached to the first axial end of the central flow tube and a first proximal arm and a second proximal arm attached to the second axial end of the central flow tube, the shunt device can be configured to be inserted into the puncture in the tissue wall such that the first axial end of the central flow tube faces a left atrium of a heart and the second axial end of the central flow tube faces a coronary sinus of the heart, and the one of the plurality of arms to which the sensor is attached can be one of the distal arms such that the sensor is positioned in the left atrium.

The plurality of arms can further include a first distal arm and a second distal arm attached to the first axial end of the central flow tube and a first proximal arm and a second proximal arm attached to the second axial end of the central flow tube; the first distal arm can form a first angle with the central axis of the central flow tube, the first angle being less than ninety degrees; the second distal arm can form a second angle with the central axis of the central flow tube, the second angle being greater than ninety degrees; and the one of the plurality of arms to which the sensor is attached can be the first distal arm.

The mating interface of the one of the plurality of arms can be angled away from the horizontal reference plane such that the sensor is angled away from the tissue wall when the shunt device is inserted in the puncture in the tissue wall.

The mating interface of the one of the plurality of arms can form an angle between 0° and 45° from the reference axis.

The angle can be about 15°-16°.

The shunt device can be configured to be inserted into the puncture in the tissue wall such that the first axial end of the central flow tube faces a left atrium of a heart and the second axial end of the central flow tube faces a coronary sinus of the heart, and the mating interface of the one of the plurality of arms can be angled into the left atrium to avoid interaction between the sensor and a mitral valve within the left atrium.

The shunt device can be configured to be inserted into the puncture in the tissue wall such that the first axial end of the central flow tube faces a left atrium of a heart and the second axial end of the central flow tube faces a coronary sinus of the heart, and the mating interface of the one of the plurality of arms can be angled into the left atrium to promote signal integrity of the sensor.

The sensor can include a pressure sensor.

The shunt device can be sterilized.

The shunt device can be formed of a shape-memory material.

The shape-memory material can be nitinol.

While the invention has been described with reference to an exemplary example(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular example(s) disclosed, but that the invention will include all examples falling within the scope of the appended claims.

1. A shunt device configured to be inserted into a puncture in a tissue wall that defines a horizontal reference plane, the shunt device comprising:
a shunt body formed of a plurality of struts, the shunt body comprising:
a central flow tube extending from a first axial end to a second axial end and defined by an opposed pair of side portions that extend laterally between an opposed pair of end portions;
a flow path extending through the central flow tube; and
a plurality of arms extending outward from the central flow tube and configured to secure the shunt device to the tissue wall, the plurality of arms including:
a first distal arm and a second distal arm attached to the first axial end of the central flow tube; and
a first proximal arm and a second proximal arm attached to the second axial end of the central flow tube; and
a secondary arm extending outward from the central flow tube and associated with at least one of the plurality of arms.

2. The shunt device of claim 1, wherein the secondary arm is connected to an end portion of the opposed pair of end portions of the central flow tube and extends radially outward with the at least one of the plurality of arms.

3. The shunt device of claim 1, wherein the secondary arm is more compliant than the at least one of the plurality of arms.

4. The shunt device of claim 3, wherein the secondary arm and the at least one of the plurality of arms are biased away from each other such that there is an axial gap between the secondary arm and the at least one of the plurality of arms when the shunt device is in a relaxed state; and
wherein the secondary arm is configured to deflect toward the at least one of the plurality of arms such that the axial gap is reduced when the shunt device is inserted in the puncture in the tissue wall and there is tissue capture by the at least one of the plurality of arms.

5. The shunt device of claim 1, wherein the secondary arm is less compliant than the at least one of the plurality of arms.

6. The shunt device of claim 5, wherein the secondary arm and the at least one of the plurality of arms are biased away from each other such that there is an axial gap between the secondary arm and the at least one of the plurality of arms when the shunt device is in a relaxed state; and
wherein the at least one of the plurality of arms is configured to deflect toward the secondary arm such that the axial gap is reduced when the shunt device is inserted in the puncture in the tissue wall and there is tissue capture by the at least one of the plurality of arms.

7. The shunt device of claim 1, wherein the central flow tube defines a central axis therethrough that is angled from a reference axis extending perpendicular through the horizontal reference plane;
wherein the first distal arm forms a first angle with the central axis of the central flow tube, the first angle being less than ninety degrees;
wherein the second distal arm forms a second angle with the central axis of the central flow tube, the second angle being greater than ninety degrees; and
wherein the secondary arm is associated with the second distal arm.

8. The shunt device of claim 1 and further comprising:
one or more tabs extending outward from the central flow tube at the first axial end and/or the second axial end and configured to prevent the shunt device from displacing through the puncture; and/or
a deflectable projection connected to at least one of the plurality of arms.

9. The shunt device of claim 1, wherein at least one of the plurality of arms includes two or more split arm portions, and/or wherein at least one of the plurality of arms includes a lengthened portion adjacent a respective terminal end.

10. The shunt device of claim 1 and further comprising:
a pressure sensor attached to the secondary arm at a mating interface such that the sensor and the secondary arm are interconnected.

11. A shunt device configured to be inserted into a puncture in a tissue wall that defines a horizontal reference plane, the shunt device comprising:
a shunt body formed of a plurality of struts, the shunt body comprising:
a central flow tube;
a flow path extending through the central flow tube; and
a plurality of arms extending outward from the central flow tube and configured to secure the shunt device to the tissue wall; and
a secondary arm associated with at least one of the plurality of arms;
wherein the secondary arm and the at least one of the plurality of arms are differently compliant;
wherein the secondary arm and the at least one of the plurality of arms are biased away from each other such that there is an axial gap between the secondary arm and the at least one of the plurality of arms when the shunt device is in a relaxed state; and
wherein a more compliant one of the secondary arm and the at least one of the plurality of arms is configured to deflect toward a less compliant one of the secondary arm and the at least one of the plurality of arms such that the axial gap is reduced when the shunt device is inserted in the puncture in the tissue wall and there is tissue capture by the at least one of the plurality of arms.

12. The shunt device of claim 11, wherein the shunt device is configured to be inserted into the puncture in the tissue wall such that a first axial end of the central flow tube faces a left atrium of a heart and a second axial end of the central flow tube faces a coronary sinus of the heart; and
wherein the secondary arm is associated with one of the plurality of arms attached to the first axial end of the central flow tube.

13. The shunt device of claim 11 and further comprising:
a pressure sensor attached to the secondary arm at a mating interface such that the sensor and the secondary arm are interconnected.

14. The shunt device of claim 11, wherein the central flow tube defines a central axis therethrough that is angled from a reference axis extending perpendicular through the horizontal reference plane;
wherein a first distal arm forms a first angle with the central axis of the central flow tube, the first angle being less than ninety degrees;
wherein a second distal arm forms a second angle with the central axis of the central flow tube, the second angle being greater than ninety degrees; and
wherein the secondary arm is associated with the second distal arm.

15. A shunt device configured to be inserted into a puncture in a tissue wall that defines a horizontal reference plane, the shunt device comprising:
a shunt body formed of a plurality of struts, the shunt body comprising:
a central flow tube;
a flow path extending through the central flow tube; and
a plurality of arms extending outward from the central flow tube and configured to secure the shunt device to the tissue wall; and
a secondary arm extending outward from the central flow tube and within a perimeter of an arm of the plurality of arms such that the secondary arm is surrounded by struts of the arm.

16. The shunt device of claim 15 and further comprising:
a pressure sensor attached to the secondary arm at a mating interface such that the sensor and the secondary arm are interconnected.

17. The shunt device of claim 15, wherein deflection of the secondary arm and/or the at least one of the plurality of arms is detectable with fluoroscopy.

18. The shunt device of claim 15, wherein the secondary arm is formed of a same material as the shunt body, wherein the secondary arm is part of a single laser cut pattern for the shunt device, and wherein the shunt device including the secondary arm is a monolithic structure.

19. The shunt device of claim 15, wherein the shunt device is formed of nitinol.

20. The shunt device of claim 15, wherein the secondary arm is more compliant than the at least one of the plurality of arms, or wherein the secondary arm is less compliant than the at least one of the plurality of arms.
